SEQUENCE LISTING (35 sequences)

SEQ ID NO: 1 - DNA, 1323 bp, Helicobacter pylori
  CDS (1)..(1323)
  misc_feature (1)..(3): Start codon encodes Met
gtgcttgtgaggttaggggttgttgcatgtcttttttggcttcattacgcttacgctacaacccttaagatcaccaatgttgtgccttttggctctagcagcgttaaaatggtgttcaatcaagaggttaaaaaattcaaagaagtttcgctcaaaaatttcaagagttatttggaattagaagccattttaaccattcctaaaaagcattaccaattctccaagcaatcgttcatcacgatcgcgcaattcagccctaagttagtgcgagtggttatcggctatgctcctaagatgacttatgaagttaaaatccttaaagacaagctttatgtttctatcgtggagaaaaagcccttaattaggcatcaaatggcgttaaaaccacccaaacaccatgcactcaaacacacaacgccaaaacccgcccataagcccattaaaaaagaggctaaaaaggttaaagaaaaaacgccaactaaacatgcgcattcaaaacacacgcattccccattgaacgaaaggagcactaaaaaagaaattcctaaaaaagaaattcctaaaaaagaagcggaaaatgagagcaagaaccaagtctttatagcagaaaaaaatgatactttcatcaaaaccaaacgcaaaaaacacaaaaagatcgttttagacgctgggcatggggggaaagattgcggggcgatgagcgcgaatttggtgtgtgaaaaagacattgttttagaagtggtgaagtttttacacaaagagcttaaaaaaagagattatagcgttttattgacaagggataaggatatttatattgatttagtggctcgcacggaattagccaataaaaaaagcgcggatttattcatctcagtgcatgccaattccatccccaaacattccacttctaacgctcatggtatagagacttattttttatccaccgcaaggagcgaaagggctaggaaagtggctgagcaagaaaataaagacgatgtgaatttaatggattatttttctaaaagtttgtttttaaattcattgaacacgcagcgattgatcgtctctaacaaattagcgattgatgtgcaatacggcatgctccaaagcgtccgcaaaaattaccctgatgtggtggatggaggcgtgagagaggggcctttttgggtgttggccggggctttaatgccttcaatcttaatagaaattggttataattcccatgcgatagaatctaaacgcatccaaagcaaaccgtatcaaaagatcttggctaagggcattgctgatggcattgatagtttcttcagcaagaatgattag

SEQ ID NO: 2 - protein, 440 aa, Helicobacter pylori
VLVRLGVVACLFWLHYAYATTLKITNVVPFGSSSVKMVFNQEVKKFKEVSLKNFKSYLELEAILTIPKKHYQFSKQSFITIAQFSPKLVRVVIGYAPKMTYEVKILKDKLYVSIVEKKPLIRHQMALKPPKHHALKHTTPKPAHKPIKKEAKKVKEKTPTKHAHSKHTHSPLNERSTKKEIPKKEIPKKEAENESKNQVFIAEKNDTFIKTKRKKHKKIVLDAGHGGKDCGAMSANLVCEKDIVLEVVKFLHKELKKRDYSVLLTRDKDIYIDLVARTELANKKSADLFISVHANSIPKHSTSNAHGIETYFLSTARSERARKVAEQENKDDVNLMDYFSKSLFLNSLNTQRLIVSNKLAIDVQYGMLQSVRKNYPDVVDGGVREGPFWVLAGALMPSILIEIGYNSHAIESKRIQSKPYQKILAKGIADGIDSFFSKND

SEQ ID NO: 3 - DNA, 1119 bp, Helicobacter pylori
  CDS (1)..(1119)
atgtcaaaaagaatgaagtgttttagtcaaaaatggttggttttttttgttacccttttattggcttctttaggccatgcgaaaatggcttttgaatccgatattgacaccaaagcgctagaggcttttggggttaatgcgggctttttatcccaaatgcccaacgctttaaaaaaaatgaataaagaagaagaatggaagagacttgtcaaaagatttgatgtgaattaccagttcatccccatcattaaaaacatgctcatagaagcgagcgtgccgcaagaatttttatttttagccatggccgagtctaaattttcatcaagggcttatagcaggaaaaaagcggtagggatttggcaattcatgccaagcacggctaaagaattagggcttaaggtcaatcattacattgatgaaagaagagatcccattaaaagcactcaagcggcgatcacttatttgaaacggctctacaagcaaaccggagagtggtatttggtcgctatggcgtataattacggcttacgcaaggttcaaaacgctattaaagccgccggcacttcggacattaaaattttgttggatgaagataagaaatacctccctaaagaaacacgagagtatatccgctccattctaagcctagcgttaaaattcaacagcctagacaacctcaaagataaagaatatctgctcaatcgtggggcgagggtgagtttagtgggcgtcccgtttaaaaggcgtgcttctttagtccaagtagccaaaaatttgaatttgagtttggaaaccttaaaatcctacaaccaccaattccgttataacattctgccttctaaagaccccacttataccatttatatcccttatgaaaaactcgctcttttcaaacaacgccagatcaaacaaaataaaaacattcaagccagttcaaaaagcccttttatcacccatgtggtcttacctaaagaaaccctatcttctatcgctaaacgctatcaagtcagtatttccaatatccaattagccaatgatctcaaagattctaatatttttatccaccagcgtttaatcatccccaccaacaaaaaattactcgctacaagggaattttaa

SEQ ID NO: 4 - protein, 372 aa, Helicobacter pylori
MSKRMKCFSQKWLVFFVTLLLASLGHAKMAFESDIDTKALEAFGVNAGFLSQMPNALKKMNKEEEWKRLVKRFDVNYQFIPIIKNMLIEASVPQEFLFLAMAESKFSSRAYSRKKAVGIWQFMPSTAKELGLKVNHYIDERRDPIKSTQAAITYLKRLYKQTGEWYLVAMAYNYGLRKVQNAIKAAGTSDIKILLDEDKKYLPKETREYIRSILSLALKFNSLDNLKDKEYLLNRGARVSLVGVPFKRRASLVQVAKNLNLSLETLKSYNHQFRYNILPSKDPTYTIYIPYEKLALFKQRQIKQNKNIQASSKSPFITHVVLPKETLSSIAKRYQVSISNIQLANDLKDSNIFIHQRLIIPTNKKLLATREF

SEQ ID NO: 5 - DNA, 1683 bp, Helicobacter pylori
  CDS (1)..(1683)
atgcgtttttttaccttgttttttatcggtatgcttggcgttggtttttctcaaaccgagttaaatttaaaagatttagaaaaaaagcccgccgggatcgttagggattattatttgtggcgttatattagcgataaaaaaaccagtttagaaaacgctaaaaaagcctatgaattgactcaaaataaaaacaacgccctacaaaaggccatgcaagaaaaaggctcagacaatgcagaaaaaaaccctgatgttaaattgcctgaagatatttattgcaagcaaacggctttagaaagcatgctagaaacaacagacactttccaagcaagctgcatcgctatcgctttaaaatcaaagatcagagattttgataaaatccctattgaaacccttaagcccttacaaattaaaatcaaagaggcttaccccgttctttatgaagaattagaaattttgcaaagtaagcatgtgagcgcttctttgtttaaggctaacgcgcaagtgtttagcgcgcttttcaatcatttgagttatgaaaaaaagctccaaatttttgaaaagcatatccccattaaagagttaaaccgtcttttagacgaaaattatccggcgtttaaccgcttgatctatcaggttattttagatcctaaattggatcattttaaagacgctctcactaaaagtaacgctacccacagcaacgcgcaaaccttttttattctagggattaatgaaatcttgcgcaaaaaaccctctaaagcgctcaagtattttgaacgatcagaagcggttgtcaaagacgatgatttttcaaaagacagagcgattttttggcagtatttagtttctaaaaagaaaaaaactttagaacgcctttcacaaagcccagctttaaatctctatagtctttatgcgagccgcaaactcaaaaccacgcccagttaccgcatcatttcacgcatccagaatttaagccaagaagatcctccttttaacacttatgaccctttttcgtggcaaatttttaaggaaaaaaccttgagtttgaaagatgagggcgcgtttaatgcgatgctaaaaagcctgtattatgaaaaaagcgctcctgaattgacctatcttttaagccaacgcaataaagacaagatttattattatttatccccttatgagggcattattgaatggcaaaatactgatgaaaaggctatggcgtatgcgatcgctaggcaagaaagctttttgctcccggcagtcatttcgcgctcgttcgctctggggcttatgcaaatcatgccctttaatgtagggcctttcgctaaaagccttggcatggataacattgatctaaacgacatgtttaaccccaacatcgctctcaaatttggcaattattacttgaaccatttgaaaaaagaattcaaccaccccctttttgtcgcctacgcttataacgctgggcctgggtttttaaggaggtggttagaaagttccaaacgatttaaagaaaaaaatcattttgagccatggcttagcatggagcttatgccttatagcgagactcgcatgtatggctttagggtcatgctcaattacttgatttatcaagaaatttttgggaatttcatccctattgatggatttttagaacaaactcttaactcaaaggacaaaccatga

SEQ ID NO: 6 - protein, 560 aa, Helicobacter pylori
MRFFTLFFIGMLGVGFSQTELNLKDLEKKPAGIVRDYYLWRYISDKKTSLENAKKAYELTQNKNNALQKAMQEKGSDNAEKNPDVKLPEDIYCKQTALESMLETTDTFQASCIAIALKSKIRDFDKIPIETLKPLQIKIKEAYPVLYEELEILQSKHVSASLFKANAQVFSALFNHLSYEKKLQIFEKHIPIKELNRLLDENYPAFNRLIYQVILDPKLDHFKDALTKSNATHSNAQTFFILGINEILRKKPSKALKYFERSEAVVKDDDFSKDRAIFWQYLVSKKKKTLERLSQSPALNLYSLYASRKLKTTPSYRIISRIQNLSQEDPPFNTYDPFSWQIFKEKTLSLKDEGAFNAMLKSLYYEKSAPELTYLLSQRNKDKIYYYLSPYEGIIEWQNTDEKAMAYAIARQESFLLPAVISRSFALGLMQIMPFNVGPFAKSLGMDNIDLNDMFNPNIALKFGNYYLNHLKKEFNHPLFVAYAYNAGPGFLRRWLESSKRFKEKNHFEPWLSMELMPYSETRMYGFRVMLNYLIYQEIFGNFIPIDGFLEQTLNSKDKP

SEQ ID NO: 7 - DNA, 37 bp, Helicobacter pylori
gaugaugauggtaccaaggattttaacttcataagtc

SEQ ID NO: 8 - DNA, 42 bp, Helicobacter pylori
aucaucaucggatccaacacgcagcgattgatcgtctctaac

SEQ ID NO: 9 - DNA, 26 bp, Helicobacter pylori
atgaaatttttggatcaagaaaaaag

SEQ ID NO: 10 - DNA, 45 bp, Helicobacter pylori
cacaagcactacaaattaacctccattgaaatagatgtgcgctgc

SEQ ID NO: 11 - DNA, 24 bp, Helicobacter pylori
gagggttaatttgtagtgcttgtg

SEQ ID NO: 12 - DNA, 23 bp, Helicobacter pylori
ctaatcattcttgctgaagaaac

SEQ ID NO: 13 - DNA, 24 bp, Helicobacter pylori
tcacaaccaagtaatcgcatcaac

SEQ ID NO: 14 - DNA, 62 bp, Helicobacter pylori
gtttcttcagcaagaatgattagtacctggagggaataatgcaatgctatatcgctgtgggg

SEQ ID NO: 15 - DNA, 30 bp, Helicobacter pylori
cggggtacctacatgcaaaatctctatccg

SEQ ID NO: 16 - DNA, 30 bp, Helicobacter pylori
cgcggatccgtgtggtaacaactcgctggg

SEQ ID NO: 17 - DNA, 43 bp, Helicobacter pylori
cggggatccgagggttaatttgtagtgcttgtgaggttagggg

SEQ ID NO: 18 - DNA, 40 bp, Helicobacter pylori
cgggtaccctaatcattcttgctgaaaaactatcaatgcc

SEQ ID NO: 19 - DNA, 40 bp, Helicobacter pylori
ataagaatgcggccgcatgcgttattttcttgtagttttc

SEQ ID NO: 20 - DNA, 41 bp, Helicobacter pylori
gttagtcacccgggtactgactttcatatctagccatgggg

SEQ ID NO: 21 - DNA, 28 bp, Helicobacter pylori
ggaattcaattcgcatttaaagggcttg

SEQ ID NO: 22 - DNA, 39 bp, Helicobacter pylori
tacctggagggaataatggactacatccttaaaaacgcc

SEQ ID NO: 23 - DNA, 37 bp, Helicobacter pylori; identical to SEQ ID NO: 7

SEQ ID NO: 24 - DNA, 42 bp, Helicobacter pylori; identical to SEQ ID NO: 8

SEQ ID NO: 25 - DNA, 30 bp, Helicobacter pylori; identical to SEQ ID NO: 15

SEQ ID NO: 26 - DNA, 30 bp, Helicobacter pylori; identical to SEQ ID NO: 16

SEQ ID NO: 27 - DNA, 43 bp, Helicobacter pylori; identical to SEQ ID NO: 17

SEQ ID NO: 28 - DNA, 40 bp, Helicobacter pylori; identical to SEQ ID NO: 18

SEQ ID NO: 29 - DNA, 36 bp, Helicobacter pylori
gaugaugauggtaccgtgtctgttgtttctagcatc

SEQ ID NO: 30 - DNA, 42 bp, Helicobacter pylori
aucaucaucggatccctaaacgacatgtttaaccccaacatc

SEQ ID NO: 31 - DNA, 39 bp, Helicobacter pylori
gaugaugauggtaccttttcctgctataagcccttgatg

SEQ ID NO: 32 - DNA, 43 bp, Helicobacter pylori
aucaucaucggatcccttggaaaccttaaaatcctacaaccac

SEQ ID NO: 33 - DNA, 1398 bp, ARTIFICIAL
  M15-pREP4-PEQ30-772 Accession Number CNCM I-3443 pQE30-772
cggataacaatttcacacagaattcattaaagaggagaaattaactatgagaggatcgcatcaccatcaccatcacggatccaccaatgttgtgccttttggctctagcagcgttaaaatggtgttcaatcaagaggttaaaaaattcaaagaagtttcgctcaaaaatttcaagagttatttggaattagaagccattttaaccattcctaaaaagcattaccaattctccaagcaatcgttcatcacgatcgcgcaattcagccctaagttagtgcgagtggttatcggctatgctcctaagatgacttatgaagttaaaatccttaaagacaagctttatgtttctatcgtggagaaaaagcccttaattaggcatcaaatggcgttaaaaccacccaaacaccatgcactcaaacacacaacgccaaaacccgcccataagcccattaaaaaagaggctaaaaaggttaaagaaaaaacgccaactaaacatgcgcattcaaaacacacgcattccccattgaacgaaaggagcactaaaaaagaaattcctaaaaaagaaattcctaaaaaagaagcggaaaatgagagcaagaaccaagtctttatagcagaaaaaaatgatactttcatcaaaaccaaacgcaaaaaacacaaaaagatcgttttagacgctgggcatggggggaaagattgcggggcgatgagcgcgaatttggtgtgtgaaaaagacattgttttagaagtggtgaagtttttacacaaagagcttaaaaaaagagattatagcgttttattgacaagggataaggatatttatattgatttagtggctcgcacggaattagccaataaaaaaagcgcggatttattcatctcagtgcatgccaattccatccccaaacattccacttctaacgctcatggtatagagacttattttttatccaccgcaaggagcgaaagggctaggaaagtggctgagcaagaaaataaagacgatgtgaatttaatggattatttttctaaaagtttgtttttaaattcattgaacacgcagcgattgatcgtctctaacaaattagcgattgatgtgcaatacggcatgctccaaagcgtccgcaaaaattaccctgatgtggtggatggaggcgtgagagaggggcctttttgggtgttggccggggctttaatgccttcaatcttaatagaaattggttataattcccatgcgatagaatctaaacgcatccaaagcaaaccgtatcaaaagatcttggctaagggcattgctgatggcattgatagtttcttcagcaagaatgattagctgcagccaagcttaattagctgagcttggactcctgttgatagatccagtaatgacctcagaac

SEQ ID NO: 34 - DNA, 2262 bp, ARTIFICIAL
  TG1-pGEX-Slt Accession Number CNCM I-3444 Plasmid pGEXSlt
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccgttagggattattatttgtggcgttatattagcgataaaaaaaccagtttagaaaacgctaaaaaagcctatgaattgactcaaaataaaaacaacgccctacaaaaggccatgcaagaaaaaggctcagacaatgcagaaaaaaaccctgatgttaaattgcctgaagatatttattgcaagcaaacggctttagaaagcatgctagaaacaacagacactttccaagcaagctgcatcgctatcgctttaaaatcaaagatcagagattttgataaaatccctattgaaacccttaagcccttacaaattaaaatcaaagaggcttaccccgttctttatgaagaattagaaattttgcaaagtaagcatgtgagcgcttctttgtttaaggctaacgcgcaagtgtttagcgcgcttttcaatcatttgagttatgaaaaaaagctccaaatttttgaaaagcatatccccattaaagagttaaaccgtcttttagacgaaaattatccggcgtttaaccgcttgatctatcaggttattttagatcctaaattggatcattttaaagacgctctcactaaaagtaacgctacccacagcaacgcgcaaaccttttttattctagggattaatgaaatcttgcgcaaaaaaccctctaaagcgctcaagtattttgaacgatcagaagcggttgtcaaagacgatgatttttcaaaagacagagcgattttttggcagtatttagtttctaaaaagaaaaaaactttagaacgcctttcacaaagcccagctttaaatctctatagtctttatgcgagccgcaaactcaaaaccacgcccagttaccgcatcatttcacgcatccagaatttaagccaagaagatcctccttttaacacttatgaccctttttcgtggcaaatttttaaggaaaaaaccttgagtttgaaagatgagggcgcgtttaatgcgatgctaaaaagcctgtattatgaaaaaagcgctcctgaattgacctatcttttaagccaacgcaataaagacaagatttattattatttatccccttatgagggcattattgaatggcaaaatactgatgaaaaggctatggcgtatgcgatcgctaggcaagaaagctttttgctcccggcagtcatttcgcgctcgttcgctctggggcttatgcaaatcatgccctttaatgtagggcctttcgctaaaagccttggcatggataacattgatctaaacgacatgtttaaccccaacatcgctctcaaatttggcaattattacttgaaccatttgaaaaaagaattcaaccaccccctttttgtcgcctacgcttataacgctgggcctgggtttttaaggaggtggttagaaagttccaaacgatttaaagaaaaaaatcattttgagccatggcttagcatggagcttatgccttatagcgagactcgcatgtatggctttagggtcatgctcaattacttgatttatcaagaaatttttgggaatttcatccctattgatggatttttagaacaaactcttaactcaaaggacaaaccatga

SEQ ID NO: 35 - DNA, 1802 bp, ARTIFICIAL
  TG1-pGEX-MltD Accession Number CNCM I-3445 Plasmid  pGEXMltD
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccgaatccgatattgacaccaaaccgctagaggcttttggggttaatgcgggctttttatcccaaatgcccaacgctttaaaaaaaatgaataaagaagaagaatggaagagacttgtcaaaagatttgatgtgaattaccagttcatccccatcattaaaaacatgctcatagaagcgagcgtgccgcaagaatttttatttttagccatggccgagtctaaattttcatcaagggcttatagcaggaaaaaagcggtagggatttggcaattcatgccaagcacggctaaagaattagggcttaaggtcaatcattacattgatgaaagaagagatcccattaaaagcactcaagcggcgatcacttatttgaaacggctctacaagcaaaccggagagtggtatttggtcgctatggcgtataattacggcttacgcaaggttcaaaacgctattaaagccgccggcacttcggacattaaaattttgttggatgaagataagaaatacctccctaaagaaacacgagagtatatccgctccattctaagcctagcgttaaaattcaacagcctagacaacctcaaagataaagaatatctgctcaatcgtggggcgagggtgagtttagtgggcgtcccgtttaaaaggcgtgcttctttagtccaagtagccaaaaatttgaatttgagtttggaaaccttaaaatcctacaaccaccaattccgttataacattctgccttctaaagaccccacttataccatttatatcccttatgaaaaactcgctcttttcaaacaacgccagatcaaacaaaataaaaacattcaagccagttcaaaaagcccttttatcacccatgtggtcttacctaaagaaaccctatcttctatcgctaaacgctatcaagtcagtatttccaatatccaattagccaatgatctcaaagattctaatatttttatccaccagcgtttaatcatccccaccaacaaaaaattactcgctacaagggaattttaaacccgggtcgactcgagcggccgcatcgtgactgactgacgatctgcctcgcgcgtttcggtgatgacggtgaaaacctctgacacatgcagctcccg

(19) United States
(12) Patent Application Publication
(10) Pub. No.: US 2007/0042448 A1
(43) Pub. Date: Feb. 22, 2007

(54) Use of enzymes from Helicobacter pylori as therapeutical targets

(75) Inventors: Ivo Boneca, Vitry sur Seine (FR); Catherine Chaput, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUTE PASTEUR, Paris Cedex (FR)

(21) Appl. No.: 11/445,300

(22) Filed: Jun. 2, 2006

Related U.S. Application Data
(60) Provisional application No. 60/686,404, filed on Jun. 2, 2005.

(51) Int. Cl. G01N 33/554; C12Q 1/18

(52) U.S. Cl. 435/7.32; 435/32

(57) ABSTRACT

Methods for identifying molecules which inhibit the virulence or pathogenicity of Helicobacter pylori by modulating the activity of hydrolases encoded by genes amiA, mltD and slt. Compositions and diagnostic and treatment methods using hydrolases and molecules which inhibit them.

### CROSS-REFERENCE TO RELATED APPLICATIONS

This application claims priority under 35 U.S.C. 119(e) to U.S. provisional application No. 60/686,404, which is hereby incorporated by reference.

### BACKGROUND OF THE INVENTION

1. Field of the Invention

Helicobacter pylori virulence genes, such as slt, mltD and amiA genes encoding hydrolases. Methods for using these genes and gene products as targets to identify drugs and other biological products which modulate H. pylori virulence.

2. Description of Related Art

Helicobacter pylori is a human pathogen responsible for gastric diseases such as duodenal ulcers and gastric adenocarcinomas. Despite a vigurous immune response, H. pylori is capable to persist for decades in its human host. H. pylori is found in biopsies under two distinct forms, a spiral-rod form and a coccoid form. Helicobacter pylori colonizes around half of the human population. Despite its medical importance, only a fragmented knowledge exists with regard to the physiology of this important pathogen. The emergence of resistant strains to most available antibiotics active against H. pylori has stimulated the search for new therapeutic strategies against H. pylori.

Screening methods such as high-throughput screening of candidate molecules are known in the art. U.S. Pat. No. 6,770,451 describes a method for screening enzyme inhibitors, U.S. Pat. No. 6,368,789 describes a method for identifying telomerase inhibitors and U.S. Pat. No. 6,051,373 describes methods for screening inhibitors of the transcription-enhancing activity of the X protein of hepatitis B virus. The screening methods and chemical libraries disclosed by these patents are hereby incorporated by reference.

Inflammatory and immunological mechanisms associated with Helicobacter pylori infection are described for example by Ferrero et al., Mol. Immunol. 42: 879-885 (2005), which is hereby incorporated by reference. Conventional diagnosis and treatment of H. pylori infection as well as antibacterial compounds useful for treating infection are described by Nakayama et al., Expert. Rev. Antiinfect. Ther. 2(4):599-610 (2004), which is hereby incorporated by reference.

### BRIEF DESCRIPTION OF THE INVENTION

The amiA, mltD and slt genes of Helicobacter pylori were previously determined not to be essential for the growth of this bacterium in vitro. Surprisingly, the present inventors have found that the amiA gene encodes an N-acetyl-muramoyl-L alanine amidase and that the mltD and slt genes encoded lytic transglycosylases which are essential for the survival of Helicobacter pylori in vivo in its ecological niche, the stomach.

One aspect of the invention is a method for identifying a compound that modulates the activity of the polypeptide hydrolases encoded by the amiA, mltD and slt genes. For example, a compound that inhibits the activity of these hydrolases can slow the growth of H. pylori and reduce or eliminate disease pathogenicity. Such a compound may also be selected to reduce particular immunological or inflammatory responses or for its ability to work synergistically with another antibacterial compound or drug, such as an antibiotic.

Thus, another aspect of the invention is the identification of compounds that eliminate or diminish inflammation or which modulate biochemical or immune responses in a subject infected with H. pylori, for example, by inhibiting peptidoglycan processing and degradation in H. pylori and consequently preventing the formation of fragments of peptidoglycan known as being involved in inflammatory diseases.

Other aspects of the invention, such as the isolation of peptidoglycan products having particular functional activities, or other diagnostic or therapeutic products, such as Bulgecin-like compounds, and other compositions or applications will be evident from the disclosure below.

### BRIEF DESCRIPTION OF THE DRAWINGS

Drawings in Section 1:

FIG. 1. Muropeptides profile of H. pylori peptidoglycan. PG from parental strain 26695 (panel A) and its ami Aisogenic mutant (panel B) were purified and digested with the muramidase M1 (Mutanolysin). The generated muropeptides were separated by HPLC. The HPLC profiles of strain 26695 (panel A) and its ami Aderivative (panel B) muropeptides composition correspond to bacteria after 8 h, 24 h and 48 h of growth. Each peak structure was assigned by MALDI-TOF mass spectrometry and corresponds to a different muropeptide: 1) GM-tripeptides, 2) GM-tetrapeptides, 3) GM-tetrapeptide-glycine, 4) GM-dipeptides, 5) GMpentapeptide. Dimers were then eluted: 6) GM-tetrapeptide-tripeptide-MG, 7) GM-tetrapeptide-tetrapeptide-glycine-MG, 8) GM-tetrapeptide-tetrapeptide-MG, 9) GM-tetrapeptide-pentapeptide-MG. Finally, anhydro-muropeptides were eluted: 10) GanhMpentapeptide, 11) and 12) GanhM-tetrapeptide-tripeptide-MG, 13) and 14) GanhM-tetrapeptide-tetrapeptide-MG, 15) GanhM-tetrapeptide-pentapeptide-MG.

FIG. 2. Morphologies of H. pylori. Scanning electron microscopy of H. pylori during exponential phase growth (4 h of culture, panels a, d and e) and after 1 week of culture of (panels c, d and f) the parental strain 26695 (panels a, b and c) and the ami A mutant (panels d, e and f). Panels g and h show transmission electron microscopy sections of the ami A mutant after ruthenium staining. The ami A mutant is able to form a complete septum without final daughter cell separation. Chains of the ami A mutant contained up to 30-40 bacteria.

FIG. 3. Effect of amoxicillin on H. pylori morphology. Scanning electron microscopy of H. pylori strain 26695 (A and B) and its isogenic ami A mutant (C and D) without (A and C) after with 3-4 h exposure to 10 μg/ml of amoxicillin (B and D). Amoxicillin treament of the ami A mutant bypasses the requirement of ami A for the morphological transition indicating that absence of coccoid forms was not due to sterical hindrance of the bactrial chains.

FIG. 4. hNod1- and hNod2-dependent activation of NF-κB by H. pylori PG. PG samples from strain NCT11637, 26695 and its isogenic ami A mutant prepared after 8 h and 48 h of growth, were digested with (M1) mutanolysin to generate muropeptides and used to stimulate human Nod1 (A) and human Nod2 (B). PG samples were also digested with recombinant Slt70 from E. coli to generate anhydromuropeptides, used to stimulate human Nod1 and human Nod2 (C), and compared to M1 generated muropeptides. Human Nod1 and Nod2 agonists were used at 10 nM and PGs at 0.3 μg/ml. Finally, purified GM-dipeptide and its anhydrous derivative G(anh)M-dipeptide were also tested for their ability to stimulate human Nod2 (D). H. pylori at different growth stages (spiral vs coccoid) and different MOI were used to stimulate the HEK293T cells and NF-β activation was determined (panel E). The same experiment was performed with the AGS gastric epithelial cell line and IL-8 secretion was determined (F). TNF-a (20 ng/ml) was used as a positive control.

FIG. 5 shows muropeptide composition of different strains at 8 and 48 hrs.

FIG. 6 depicts graphically alternative PG hydrolase mechanisms.

FIG. 7 shows muropeptide composition of slt, mltD, HP1118 and HP0087 mutant strains.

FIG. 8 compares the specific activity of MurE of strains 26695 and 26695 amiA⁻.

FIG. 9 diagrams introduction of wild-type amiA at different loci.

FIG. 10 shows a chromatogram of the Slt70 digested PG of H. pylori.

Drawings in Section 2:

FIG. 11 shows HPLC chromatograms of H. pylori muropeptide composition (A) and distribution of glycan chain length (B). Muropeptide peaks (from 1 to 15) correspond to the nomenclature in Table 1. Glycan strand peaks (from 1 to 25 and >26) correspond to the nomenclature in Table 2. Comparative analysis of strains 26695 and 26695 ami A is presented in Tables 1 and 2 for the muropeptide and glycan chain distribution, respectively.

FIG. 12. Electron microscopy of wild type H. pylori strain X47-2AL (A and B) and its isogenic ami A mutant (C to F). Panel C shows the chaining phenotype of the ami A mutants. Arrows heads highlight flagella located in the middle of a bacterial chain. Examples of higher magnifications of flagella of the ami A mutant are illustrated in panels D to F. Panel D shows polar flagella and panels E and F illustrate flagella at division sites.

FIG. 13. Mice colonization with wild type X47-2AL and its isogenic ami A mutants after 3, 15 and 30 days of infections. For each experiment, the inventors used an even mixture of three independent clones of the ami A mutants. Since the ami A mutant chains, the inventors considered it was plausible that the inventors were not able to detect colonization of the mutant using a low infectious dose. Therefore, a higher dose was also used. The ami A mutant was still unable to colonize C57/BL6J mice.

FIG. 14. Schematic representation of the PG layer of H. pylori considering the 3-for-1 model (A), the scaffold model (B) and modified scaffold model (C). The peptidoglycan layer is represented schematically seen from the top, from the pole and from a cross-section of the bacteria of the long axis. Black and red lines represent glycan strands and stem peptides, respectively. Filled circles correspond to glycan strands, which are perpendicular to the cytoplasmic membrane.

Drawings in Section 3:

FIG. 15 Schematic representation of the genomic regions surrounding the genes slt and mltD in strain 26695. By PCR analysis, the inventors have confirmed the conservation of the two regions in different H. pylori strains.

FIG. 16. Silver staining of extracted LPS from different H. pylori strains and the slt mutants. While the miniTn3-Km transposon insertion into the slt gene generated a polar effect on the gaIU gene resulting in a rough LPS phenotype, the non-polar K2 cassette did not affect the smooth LPS genotype of the N6 strain. Inactivation of the mltD gene had no effect on the LPS phenotype.

FIG. 17. Impact of the polar effect on the miniTn3-Km transposon on the morphology of H. pylori mltD mutant. Insertion of the transposon in the mltD gene resulted in a chaining phenotype probably due to a polar effect on hp1567 encoding a putative GTPase. Inactivation of mltD with the non-polar K2 cassette had no effect on the mutant morphology reinforcing the non-polar nature of the K2 cassette. An slt.K2 mutant had a normal morphology.

FIG. 18. Growth curve of H. pylori 26695 and its slt and mltD mutants. Growth in liquid culture was followed by optical density (600 nm) and bacterial viability was monitored by counting the number of colony forming units (A). Growth experiments were done six times (panel A is a representative experiment). The three strains presented an identical growth rate. Survival in stationary phase was enhanced for the mltD strain as illustrated by a slower death rate (B). mltD takes in average 66 minutes for half of the population to die, while half of the population of both the 26695 strain and the slt mutant dies in average in 32-34 minutes.

FIG. 19. Glycan strand length distribution of the parental strain 26695 compared to the slt mutant (A) and the mltD mutant (B). Each glycan strand species is represented as percentage of the total UV absorbing material (FIG. 21). In panel C, each glycan strand species is represented as a molar percentage taking into account each species individual abundance. Panel C shows that the majority of the H. pylori glycan strands are short strands and that both mutants have a decrease amount of short glycan strands. Furthermore, the slt mutant is characterized by an almost complete absence of GanhM disaccharide (peak 1 in supplementary FIG. 1).

FIG. 20. Digestion of H. pylori PG with the exo-type lytic transglycosylase Slt70 from E. coli. H. pylori PG was digested either completely with Slt70 (2 days) or for brief periods (1 and 5 minutes). Each peak was collected, desalted and analyzed by MALDI-TOF mass spectrometry to confirm the muropeptide nature of each peak. Peaks 1 to 9 correspond to GanhMtripeptide, GanhM-tetrapeptide, GanhM-tetrapeptide-glycine, GanhM-pentapeptide, GanhM-tri-tetra-GanhM, GanhM-tetra-tetra-glycine-GanhM and GanhM-tetra-tetra-GanhM, GanhM-penta-tetra-GanhM, respectively.

FIG. 21. Analysis of the glycan strand length distribution of the parental strain 26695 and its slt and mltD single mutants. The peak number corresponds to the number of disaccharide repeating units of each glycan strand species. Glycans with more than 26 disaccharide repeating units are eluted as a single peak at the end of the chromatogram by a single 30% acetonitrile step. Note that the scale of the left and right Y axis is different to accommodate the single peak at the end of the chromatogram. The relative intensity of each peak as presented in FIG. 5 corresponds to the ration of each peak area over the total UV glycan strand peak area. The relative percentage of the single peak of the glycan strands >26 disaccharide repeating units is presented to the right of the corresponding peak.

The patent or application file contains at least one drawing executed in color. Copies of this patent or patent application publication with color drawing(s) will be provided by the office upon request and payment of the necessary fee.

### DETAILED DESCRIPTION OF THE PREFERRED EMBODIMENTS

A compound that modulates the activity of a polypeptide hydrolase, such as lytic transglycosylases, encoded by the mltD and slt genes of H. pylori, or N-acetylmuramoyl-L-alanylamidase encoded by amiA, may be identified by contacting a test compound with a polypeptide expressed by an amiA, mltD and slt gene and determining the effect that the test compound has on the activity of the encoded polypeptide or on the biochemistry of H. pylori as modulated by that polypeptide. Such effects may be determined in vitro or in vivo.

A screening method, such as the high-throughput methods described above, may evaluate the effects of a test substance on a cell (“cell based screening”) or its effects on an isolated gene or gene product, such as a polypeptide or enzyme (“biochemical-based screening”).

Cell based screening uses intact or viable cells, while biochemical-based screening may use isolated or partially isolated components of an organism, e.g., an isolated AmiA, Slt or MltD polypeptide, or active polypeptide fragment. Cellular based assays may employ cells designed to modulate the expression or function of particular genes, such as the amiA, slt or mltD genes, so as to optimize sensitivity of a screening process. These genes need not necessarily be expressed in H. pylori cells, but may be placed in other conventional cell lines, including E. coli, which can then be used to evaluate the effects of test substances on gene activity or on the activity of the expressed products. Methods of expressing genes in host cells are well known and are also incorporated by reference to Current Protocols in Molecular Biology (May, 2006).

Screening processes usually involve comparing the effects of a control which is not contacted with a test substance, with those obtained from a test sample exposed to a test substance. The ability to modulate particular cellular or biochemical activities is comparatively measured. The term modulate includes measuring the ability of a test substance to suppress, enhance, induce or shut down a cellular or biochemical function.

Screening processes may also measure the direct or secondary effects of modulating activities associated with genes such as amiA, slt or mltD on other cellular processes or structures. For example, modulating a certain cell process may result in the accumulation of certain biochemical products or intermediates, or in the reduction of others. Similarly, modulation may have phenotypical effects on cell morphology as determined by microscopy or electron microscopy. Many ways of measuring modulatory effects of test substances are exemplified below and, while screening processes are not limited to these particular exemplified methods, any of these may be selected as a basis for a screening assay by one with skill in the art.

Methods for identifying inhibitors of bacterial hydrolases, such as transglycosylase inhibitors, are known in the art and are hereby incorporated by reference to Ravishankar, S., Kumar, V. P., Chandrakala, B., Jha, R. K., Solapure, S. M., de Sousa, S. M., “Scintillation proximity assay for inhibitors of Escherichia coli MurG and, optionally, MraY”, Antimicrob. Agents Chemother. 49(4):1410-8 (2005); and Branstrom, A. A., Midha, S., Goldman, R. C., “In situ assay for identifying inhibitors of bacterial transglycosylase”, FEMS Microbiol. Lett. 191(2):187-90 (2000).

Inhibitors of bacterial hydrolases, such as Bulgecin are known. These inhibitors as well as methods for identifying them are incorporated by reference to:

Simm A M, Loveridge E J, Crosby J, Avison M B, Walsh T R, Bennett P M.

Bulgecin A: a novel inhibitor of binuclear metallo-beta-lactamases.
Biochem. J. May 1, 2005; 387(Pt 3):585-90.
   Khalaf J K, Datta A.
An efficient and highly stereocontrolled route to bulgecinine hydrochloride.
J. Org. Chem. Jan. 23, 2004; 69(2):387-90.
   Heidrich C, Temiplin M F, Ursinus A, Merdanovic M, Berger J, Schwarz H, de Pedro M A, Holtje J V.
Involvement of N-acetylmuramyl-L-alanine amidases in cell separation and antibiotic-induced autolysis of Escherichia coli.
Mol Microbiol. July 2001; 41(1):167-78.
   van Asselt E J, Kalk K H, Dijkstra B W.
Crystallographic studies of the interactions of Escherichia coli lytic transglycosylase Slt35 with peptidoglycan.
Biochemistry. Feb. 29, 2000;39(8):1924-34.
   Kraft A R, Prabhu J, Ursinus A, Holtje J V.
Interference with murein turnover has no effect on growth but reduces beta-lactamase induction in Escherichia coli.
J. Bacteriol. December 1999; 181(23):7192-8.
   Karlsen S, Hough E.
Structure of a complex between bulgecin, a bacterial metabolite, and lysozyme from the rainbow trout.
Acta Crystallogr. D. Biol. Crystallogr. Jan. 1, 1996;52(Pt 1):115-23.
   Karlsen S, Hough E, Rao Z H, Isaacs N W.
Structure of a bulgecin-inhibited g-type lysozyme from the egg white of the Australian black swan. A comparison of the binding of bulgecin to three muramidases.
Acta Crystallogr. D. Biol. Crystallogr. Jan. 1, 1996;52(Pt 1):105-14.
   Thunnissen A M, Rozeboom H J, Kalk K H, Dijkstra B W.
Structure of the 70-kDa soluble lytic transglycosylase complexed with bulgecin A. Implications for the enzymatic mechanism.
Biochemistry. Oct. 3, 1995;34(39): 12729-37.
   Romeis T, Vollmer W. Holtje J V.
Characterization of three different lytic transglycosylases in Escherichia coli.
FEMS Microbiol. Lett. Aug. 1, 1993;111(2-3):141-6.
   Templin M F, Edwards D H, Holtje J V.
A murein hydrolase is the specific target of bulgecin in Escherichia coli.
J. Biol. Chem. Oct. 5, 1992;267(28):20039-43.
   Gwynn M N, Box S J, Brown A G, Gilpin M L.
MM 42842, a new member of the monobactam family produced by Pseudomonas cocovenenans. I. Identification of the producing organism.
J. Antibiot. (Tokyo). January 1988;41(1):1-6.
   Nakao M, Yukishige K. Kondo M, Imada A.
Novel morphological changes in gram-negative bacteria caused by combination of bulgecin and cefinenoxime.
Antimicrob. Agents Chemother. September 1986;30(3):414-7.
   Parker W L, O'Sullivan J, Sykes R B.
Naturally occurring monobactams.
Adv. Appl. Microbiol. 1986;31:181-205. Review.
   Shinagawa S, Maki M, Kintaka K, Imada A, Asai M.
Isolation and characterization of bulgecins, new bacterial metabolites with bulge-inducing activity.
J. Antibiot. (Tokyo). January 1985;38(1):17-23.
   Imada A, Kintaka K, Nakao M, Shinagawa S.
Bulgecin, a bacterial metabolite which in concert with beta-lactam antibiotics causes bulge formation.
J. Antibiot. (Tokyo). October 1982;35(10):1400-3.

Templin et al. (1992), page 20039, describe the structure of bulgecins and this structure is specifically incorporated by reference.

Bulgecin-like compounds may be used to inhibit the growth of H. pylori and treat infection since the present inventors have found that expression of hydrolase genes is essential for H. pylori survival in vivo. Moreover, Bulgecin or Bulgecin-like compounds may be used as positive controls in assays of test compounds while determining their activity on bacterial hydrolases.

Large numbers of test compounds may be efficiently screened for their ability to bind to and/or modulate the activity of the hydrolases encoded by H. pylori genes. While generally a test compound will be selected for its ability to inhibit expression of a hydrolase and thus attenuate bacterial virulence, it may also be selected for its ability to increase the expression or activity of a H. pylori hydrolase.

Test compounds may have different structures. For example, the test compounds may be small organic molecules having a molecular mass of about 50 to 2,500 daltons, molecules containing metal ions, steroids, carbohydrates, bulgecin-like compounds or compounds that bind to the same determinants that bulgecin does, saccharides, glycolipids, lipids and lipopeptides, peptides having less than 100 residues, polypeptides, cytokines or cytokine fragments, peptide hormone or peptide hormone fragments, digestive enzymes, receptor proteins, subunits or fragments thereof, antibodies or antibody fragments, and molecules or other products isolated from natural sources such as from bacteria, fungi, parasites, plants, and animals.

Test molecules may also be preselected based on their known or predicted ability to be able to contact a H. pylori hydrolase under in vivo conditions, e.g., molecules that can permeate or be actively transported across gram-negative outer membrane, periplasm or inner cytoplasmic membrane or known substrates or products of hydrolases. Test molecules may also be preselected for their known or predicted abilities to affect transcription, mRNA stability, translation or post-translational folding or modification of a H. pylori hydrolase.

Screening may be performed in vitro using isolated or semi-purified hydrolases, in vitro using hydrolases expressed by recombinant host cells such as E. coli or H. pylori, or in vivo by inoculation of an animal, especially the stomach or gastric mucosa with H. pylori or another prokaryotic cell expressing the hydrolase. Suitable host cells for expression of hydrolases in the stomach or gastric mucosa can be selected based on known pathogens of the gastrointestinal system.

The effect of a test compound on hydrolase expression or activity may be determined by various means including the analysis of the peptidoglycan and peptidoglycan fragments produced by the particular combination of a peptidoglycan molecule, hydrolase and a test compound, the analysis of inflammatory responses, including IL-8 and NF-κB responses, of host cells exposed to a particular combination of bacterial cells expressing a hydrolase that have been contacted with a test compound, the cellular morphology of H. pylori after exposure to a test compound, such as a change from a spiral to coccoid form, or the ability of H. pylori to attach to, invade or colonize the stomach or gastric mucosa. The effect of inhibition of hydrolase activity may also be measured by assessing the bacteriostatic or bacteriocidal effect of the test compound.

A test compound may also be evaluated for its ability to affect cellular or humoral immune responses to infection by H. pylori. Various assays for antigen-specific immune responses are well known in the art, and those responses induced in the presence of a test compound may be compared to those induced in its absence. The effects of a test compound on surface determinants of H. pylori may be visually assessed by probing its surface with antibodies or other ligands to particular surface antigens or determinants, and comparing these determinants with those from cells not exposed to the test compound. Periplasmic and other subsurface structures may also be analyzed in a similar manner. Animals used for in vivo testing may have various genetic backgrounds, such as being homozygous for an NOD1⁻ mutation or being NOD1⁺. Methods for evaluating NOD1 associated responses are incorporated by reference to Viala, et al., Nature Immunol. 5:1166 (2004).

The binding of a test compound to a particular hydrolase, such as those encoded by amiA, mltD or slt, or to a cell expression a hydrolase, may preferably be performed under conditions similar to those found in the gut, since H. pylori is a gut pathogen.

Mutant cells, such as H. pylori containing attenuating or inactivating mutations in the amiA, mltD or slt hydrolases, may be used to produce variant peptidoglycans or may be used as attenuated organisms for the production of a vaccine or as immunogens to induce H. pylori specific cellular or humor immune responses. Conventional excipients, carriers or adjuvants may be used in combination with these hydrolases and/or organisms. Variant peptidoglycan components or variant distributions of peptidoglycan components may also be tested for their ability to inhibit or promote inflammation or for their immunological activities by well-known methods. Antibodies may be produced to specific peptidoglycan components associated with detrimental inflammatory and immunological phenomena by well known methods including hybridoma production.

The present invention is also directed to a technical platform comprising at least:
an isolated or semi-purified hydrolase
A peptidoglycan or a peptidoglycan variant, eventually labeled
All the reagents necessary to detect the peptidoglycan fragments produced upon the action of at least one H. pylori hydrolase:
   directly, by detecting the labeled fragments or peptidoglycan or
   indirectly, by measuring the inflammatory response induced by said fragments or peptidoglycan.

A compound which inhibits inflammation caused by or associated with Helicobacter pylori infection may also be identified according to the invention by contacting a subject infected with Helicobacter pylori with a test compound determined to inhibit Slt, Mlt D and/or Ami A protein activity, and measuring an inflammatory and/or immune response in said subject compared to the corresponding inflammatory and/or immune response in a control subject to which the test compound has not been administered. An inflammatory response associated with NF-κB or IL-8 may be measured. Test compounds which inhibit Slt protein activity, Mlt D protein activity and/or Ami A protein activity may be identified and isolated. Such a method may be performed in vivo or in vitro. The subject of said method may be Nod1⁻ (homozygous) or Nod1⁺.

The effects of a test compound may also be tested in the presence of an antibiotic or other drug, especially those that are active in the stomach compartment and gastric mucosa or those used to treat gastric disorders. Exemplary antibiotics and drugs include Amoxicillin, Metronidazole, Chlarithromycin and Omeprazole and antibiotics falling within the classes defined by these antibiotics. Substances may also be evaluated for their ability to modulte, particularly enhance, healing responses or antibacterial activity in the presence of drugs and biological agents used to treat diseases associated with H. pylori infection. Useful antibiotics and therapeutics are described by Nakayama et al., Expert. Rev. Antiinfect. Ther. 2(4):599-610 (2004) which has been incorporated by reference.

The invention is also directed to a method for identifying an antibacterial composition that comprises at least one antibiotic and at least one compound that inhibits the amiA, mltD or slt genes or their gene products, comprising contacting a mixture of at least one antibiotic and a test compound with Helicobacter pylori, and determining the amount of bacteriostatic or bacteriocidal activity of said composition. Bacteriostatic activity may be measured for example by determining the degree of inhibition of bacterial growth or metabolism and bacteriocidal activity by determining the number of bacterial killed. Such a method may be performed in vivo or in vitro and with subjects who are Nod1⁻ (homozygous) or Nod1⁺.

Pro-inflammatory peptidoglycan fragments may be identified or detected according to the invention by methods comprising isolating peptidoglycan or peptidoglycan fragments from Helicobacter pylori in which the expression of polypeptides from the slt, mltD and/or amiA gene(s) have been reduced or eliminated, isolating peptidoglycan or peptidoglycan fragments from Helicobacter pylori in which the expression of polypeptides from the slt, mltD and/or amiA gene(s) have not been reduced or eliminated, and comparing the immune or inflammatory responses or degree of immune or inflammatory responses induced by the two isolated peptidoglycan or peptidoglycan fragment samples. This method may involve measurement of comparative immune or pro-inflammatory responses induced by the peptidoglycan or peptidoglycan fragments in vitro. The peptidoglycan or peptidoglycan fragments may also be administered to an animal and the immune or inflammatory responses to these products determined. These products may be administered to subjects who are Nod1⁻ (homozygous) or Nod1⁺.

The invention also pertains to a recombinant Helicobacter pylori comprising an attenuation or deletion of at least one gene selected from the group consisting of slt, mltD and amiA; and to a composition comprising such a recombinant Helicobacter pylori and an adjuvant. Helicobacter pylori infection may be treated by administering such a recombinant Helicobacter pylori optionally with an adjuvant to subject infected with or in danger of developing H. pylori infection.

Isolated or purified peptidoglycan or peptidoglycan fragments may be obtained from such a recombinant Helicobacter pylori and formulated into a composition by the addition of a pharmaceutically acceptable carrier, excipient or adjuvant. Such a composition may be employed to modulating or treat a Helicobacter pylori infection by administering it to a suitable subject, such as those inflicted with gastric disorders associated with H. pylori.

The invention also pertains to a method for detecting a compound that inhibits peptidoglycan metabolism of H. pylori comprising contacting in a suitable reaction buffer a peptidoglycan hydrolase from H. pylori selected from the group consisting of Slt, MltD and AmiA, with labeled peptidoglycan and with a test compound, measuring the release of labeled peptidoglycan fragments into the reaction buffer, and comparing the amount of released peptidoglycan fragments with the amount of released peptidoglycan fragments in a control sample that does not contain the test compound. Bulgecin may be used as a positive control for inhibition of hydrolase activity and the peptidoglycan may be labeled with a radioactive isotope, a fluorescent tag, or a chromophore.

Screening methods for therapeutic compounds effective against H. pylori may also be performed by measuring the activation or expression level of the amiA, mltD or slt genes by methods well-known in the art, for example, by measuring the amount of mRNA transcribed from these genes or the amount of protein translated from one of these genes. Such methods are incorporated by reference to Current Protocols in Molecular Biology (May, 2006). Compounds which either activate or inhibit these genes are selected since they would modulate H. pylori virulence via their effects on these genes.

A candidate compound that modulates inflammatory responses induced by H. pylori may be identified by infecting a cell with H. pylori, contacting the infected cell with a test compound, and measuring at least one of activation of NK kappa B, IL-8 production levels, or the production level of pro-inflammatory or anti-inflammatory cytokines, and comparing said measured levels with those obtained from a control sample not contacted with said test compound. Such a cell may be an epithelial or monocytic cell or HEK293T cells transfected with an IgK-luciferase where the activation of NK-κB is detected by fluorescence. Alternatively, the cell may be a gastric epithelial carcinoma cell AGS and IL-8 production can be measured; or the cells are THP-1 cells and the production of pro-inflammatory and anti-inflammatory cytokines is measured.

H. pylori infection may be treated by administering an amount of an inhibitor of an H. pylori hydrolase and optionally one or more other antibacterial compounds. An example of such an inhibitor is a Bulgecin. For example, a composition comprising one or more inhibitors of an H. pylori hydrolase and a pharmaceutically acceptable carrier or excipient, and optionally one or more antibacterial compounds and/or one or more gastric medications may be administered to a subject in need of treatment.

The invention also includes a technical platform comprising an isolated or semi-Purified hydrolase selected from the group consisting of Slt, MltD and AmiA from H. pylori; a peptidoglycan, peptidoglycan fragment or peptidoglycan-like compound, which may be optionally labeled, and reagent(s) necessary to detect peptidoglycan fragments produced by the action of the hydrolase.

Peptidoglycan fragments produced by the action of an H. pylori hydrolase on a peptidoglycan sample may be detected directly by well-known biochemical methods or indirectly by measuring an inflammatory response induced by said fragments or by peptidoglycan.

A compound which inhibits the activity of a H. pylori hydrolase may be detected in vivo by infecting a mouse with a strain of H. pylori having wild-type hydrolase genes,treating the infected mice with either amoxicillin or bulgecin as positive controls and/or with no compound, and with a test compound, comparing the extent of colonization by H. pylori of the stomachs of said control mice with those of mice treated with the test compound.

A compound which inhibits H. pylori hydrolase activity also may be identified by contacting in a liquid culture medium H. pylori bacteria having wild-type amiA gene, and a test compound or a positive control compound, such as amoxicillin or bulgecin, andmicroscopically examining said cells for filaments indicating phenotype reversion in said bacteria.

In one embodiment of the present invention the hydrolases are chosen from among the polypeptides encoded by the nucleotide sequences inserted in a plasmid contained in E. coli and deposited, on Jun. 2, 2005 at the CNCM (Collection Nationale de Cultures de Microorganismes) under the Budapest Treaty and under the numbers:
Accession Number: CNCM I-3443; pQE30-772 (M15-pREP4-PQE30-772)
Accession Number: CNCM I-3444; Plasmid pGEXSlt (TG1-pGEX-Slt)
Accession Number: CNCM I-3445; Plasmid: pGEXMltD (TG1-pGEX-MltD).

Peptidoglycan assays are known in the art and the details of these assays are incorporated by reference to Bernadsky et al., J. Bacteriol. 176(17): 5225-5232 (1994), Nagata, et al., Limnol. Oceanogr. 48(2): 745-754 (2003), and Dijkstra et al., “A New Member of the Transglycosidase Family of Escherichia coli Displays a Gram-positive Hydrolase Motif”, Chapter 5, pp. 85-101. Peptidoglycan may be radiolabeled with tritium, ¹⁴C or ¹⁵N. The enzymatic processes involved in the degradation of peptidoglycan may be evaluated by the zymogram technique (polyacrylamide gel resolution of peptidoglycan). The enzymes in the supernatant of bacterial cultures which are capable of degrading peptidoglycan will produce bands of degraded peptidoglycan in the zymograms. This technique is known in the art and is incorporated by reference to Bernadsky et al., J. Bacteriol. 176: 5225-5232 (1994).

Four families of peptidoglycan lytic transglycosylases have been described and their structural and functional features, including conserved structural motifs of the enzymes and the polynucleotides encoding them, are hereby incorporated by reference to Blackburn et al., J. Mol. Evol. 52:78-84 (2001). For example, the Slt and MltD hydrolases share functional activity as well as having substantial similarity in their active domains.

SEQ ID NO: 1 depicts the polynucleotide sequence of H. pylori ami A and SEQ ID NO: 2 shows the amino acid sequence of the amidase encoded by this gene. Similarly, SEQ ID NOS: 3 and 5 respectively depict the polynucleotide sequences of the mltD and slt genes and SEQ ID NOS: 4 and 6 the corresponding lytic transhydrolases. One with skill in the molecular biological arts may of course truncate these gene sequences and isolate shorter polynucleotides which encode enzymatically active fragments of the polypeptides of SEQ ID NOS: 2, 4 and 6 according to well-known methods.

Variants of SEQ ID NOS: 1 (amiA), 3 (mltD) and 5 (slt) may be produced and screened by methods well-known in the art or by the methods described by Current Protocols in Molecular Biology (1987-2005), vols. 1-4, which is hereby incorporated by reference. A mutant or variant of the polynucleotides of SEQ ID NOS: 1, 3, and 5 will have 70%, 80%, 90%, 95%, or 99% homology or similarity to the corresponding sequence and all intermediate subranges and values. Similarly a mutant or variant of the polypeptides of SEQ ID NOS: 2 (AmiA), 4 (Mlt D), and 6 (Slt) will have 70%, 80%, 90%, 95%, or 99% homology or similarity to the corresponding amino acid sequence. Such mutants or variants may also encode, or be functionally active fragments of, these polypeptide sequences. A variant or mutant of the polynucleotide sequences of SEQ ID NOS: 1, 3, or 5 will exert, or encode a polypeptide having, one of the functional activities described herein.

Similarity or homology may be determined by an algorithm, such as those described by Current Protocols in Molecular Biology, vol. 4, chapter 19 (1987-2005) or by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Variants of SEQ ID NOS: 1, 3, and 5 may also be characterized by their ability to hybridize under stringent conditions with the complements of SEQ ID NOS: 1, 3, and 5. Alternatively, such variants may be simply isolated from other Helicobacter pylori strains. Hybridization conditions may comprise hybridization at 5×SSC at a temperature of about 50 to 68° C. Washing may be performed using 2×SSC, optionally followed by washing using 0.5×SSC. For even higher stringency, the hybridization temperature may be raised to 68° C. or washing maybe performed in a salt solution of 0.1×SSC, or both. Other conventional hybridization procedures and conditions may also be used as described by Current Protocols in Molecular Biology, (1987-2005), see e.g. Chapter 2. The particular details of the subject matter described above are incorporated by reference to the corresponding documents cited above.

The Slt, MltD and Ami hydrolases are important to motility of H. pylori. Motility is an important virulence (colonization) factor of H. pylori and methods for evaluation motility are well-known in the art, and are also incorporated by reference to O'Toole, P. W., et al., Microbes Infect. August 2000;2(10):1207-14 and Bjorkholm, B, et al., Helicobacter. September 2000;5(3):148-54. Test compounds may be screened by evaluating their effects on the hydrolases of the invention and/or their effects on motility or colonization ability of H. pylori. Compounds which inhibit or interfere with hydrolase function may be identified by the effects such inhibition or interference has on motility or colonization ability by H. pylori as determined by these well-known methods.

The Examples below show aspects of the invention, but the invention is not limited to what is shown in these Examples.

### Section 1

The inventors have discovered that AmiA plays an important role in morphological transition of H. pylori and in subsequent immune escape. Based on this discovery, it is possible to test compounds which modulate this morphological transition and attenuate virulence or pathogenicity of H. pylori.

Helicobacter pylori is a human pathogen responsible for gastric diseases such as duodenal ulcers and gastric adenocarcinomas. Despite a vigurous immune response, H. pylori is capable to persist for decades in its human host. H. pylori is found in biopsies under two distinct forms, a spiral-rod form and a coccoid form. The inventors investigated the molecular mechanisms leading to the transition of H. pylori from a spiral-rod shaped organism to a coccoid organism. The morphological transition is accompanied by modifications of the bacterial cell wall peptidoglycan.

The inventors have identified the AmiA protein as essential for this morphological transition and modification of the cell wall peptidoglycan and demonstrate that the cell wall modifications and morphological transition result in an escape of these coccoid forms from the immune system and can lead to persistence of H. pylori infection during the life time of its human host.

H. pylori PG structure during the morpholgical transition was investigated. The transition correlated with an accumulation of the N-acetyl-D-glucosaminyl-β (1,4)-N-acetylmuramyl (GM)-dipeptide motif. The inventors investigated the molecular mechanisms responsible for the GM-dipeptide motif accumulation, and studied the role of various putative PG hydrolases in this process. Interestingly, a mutant of the ami A gene, encoding a putative PG hydrolase, was impaired in accumulating the GM-dipeptide motif and the transformation into coccoids. The inventors investigated the role of the morphological transition and the PG modification in the biology of H. pylori. PG modification and transformation of H. pylori was accompanied by an escape from detection by Nod1 and the absence of NF-κB activation in epithelial cells. Accordingly, coccoids were unable to induce IL-8 secretion by AGS gastric epithelial cells. Hence, ami A is the first genetic determinant found to be required for morphological transition into the coccoid forms, which contribute to modultation of the host response and which pertain to the chronicity of H. pylori infection.

As noted above, the human gastric pathogen Helicobacter pylori is responsible for peptic ulcers and neoplasia. Both in vitro and in the human stomach it can be found in two forms, the bacillary and coccoid forms. The molecular mechanisms of the morphological transition between these two forms and the role of coccoids remains largely unknown. The peptidoglycan (PG) layer is a major determinant of bacterial cell shape. Helicobacter pylori is a human pathogen with an unique niche: the stomach. The presence of this bacterium is always associated with chronic gastritis, and less often with severe duodenal ulcers, gastric adenocarcinoma or mucosa-associated lymphoid tissue (MALT) lymphoma. H. pylori has the interesting ability to convert from bacillary to coccoid forms. The coccoid forms appear in stationary phase and can also be induced under stress conditions, for example, following modification of pH, O₂ tension or temperature [ 1,2], or exposure to antibiotics such as amoxicillin [3,4]. However, the biological role of this form is still controversial. Both forms are commonly observed in the human stomach [5,6]. Coccoids are viable but non cultivable, and this has led to the suggestion that the coccoid form is of the persisting form, allowing H. pylori to spread between human hosts. Coccoid forms contain a reasonable quantity of ATP [7] and an active respiratory chain [8-10]; they are also viable as assessed by viability staining [11,12,13,14]. Various proteins (including VacA and CagA) and activities (for example urease activity) are detectable, but it is not clear whether there is any de novo protein synthesis [15]. Despite interest in this subject, little is known about the process of morphological transition into coccoid forms. Proteome and transcriptome analyses failed to identify proteins determinant in the transition [7,16-19]. The cdrA gene was implicated in coccoid formation [20]. These results are controversial since the cdrA egene is inactivated in several strains including the two sequenced strains 26695 and J99. Hence, CdrA is unlikely to have a major role if any in coccoid formation. It is known, however, that the lipid composition of H. pylori changes substantially during the transition into coccoid forms [21].

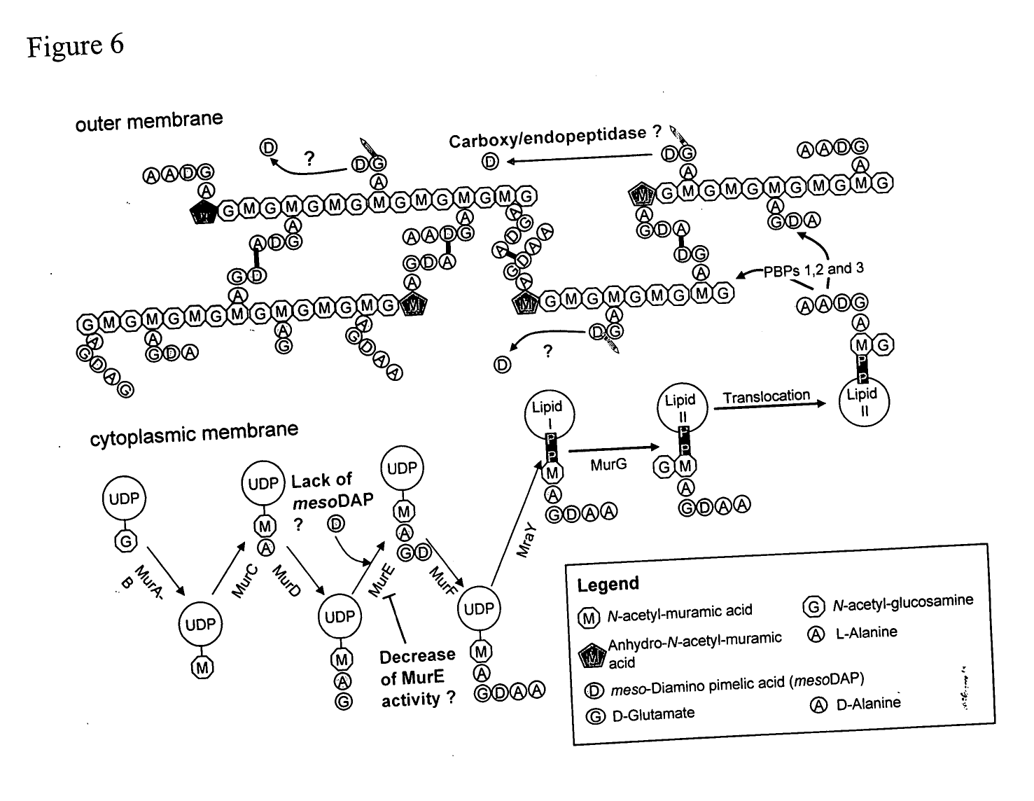

One of the main determinants of bacterial shape is the peptidoglycan (PG) layer (for a recent review see reference [22]). Costa et al. [23] implicated a modification of the muropeptides composition of H. pylori PG with the transition from bacillary to the coccoid form: the N-acetyl-D-glucosaminyl-β(1,4)-N-acetylmuramyl-L-Ala-D-Glu (GM-dipeptide) motif accumulated in the sacculus after 2 days of liquid culture. This motif lacks the diamino acid, meso-diaminopimelic acid, required for PG transpeptidation. Possibly, a looser PG macromolecule could explain the shape transition of H. pylori from spiral to coccoid. Here, the inventors studied the genetic determinants involved in the accumulation of the GM dipeptide motif. Several alternative mechanisms could explain this phenomenon (see the supplementary material and FIG. 6), and PG hydrolases could be involved.

The inventors describe the construction of a mutant of the ami A gene, encoding a putative PG hydrolase, which is impaired in the accumulation of the GM-dipeptide motif; it is also defective in the transition from spiral bacteria into coccoid forms. The inventors show that this phenotype (morphological transition and PG modifications) is associated with impaired sensing by the Nod1 pathway, impaired activation of NF-κB and impaired cytokine production by AGS gastric epithelial cells. The inventors thus identified a new mechanism for bacterial escape from the innate immune system.

### EXAMPLE 1

### Accumulation of the GM-Dipeptide Motif in the PG of Various Strains of H. pylori

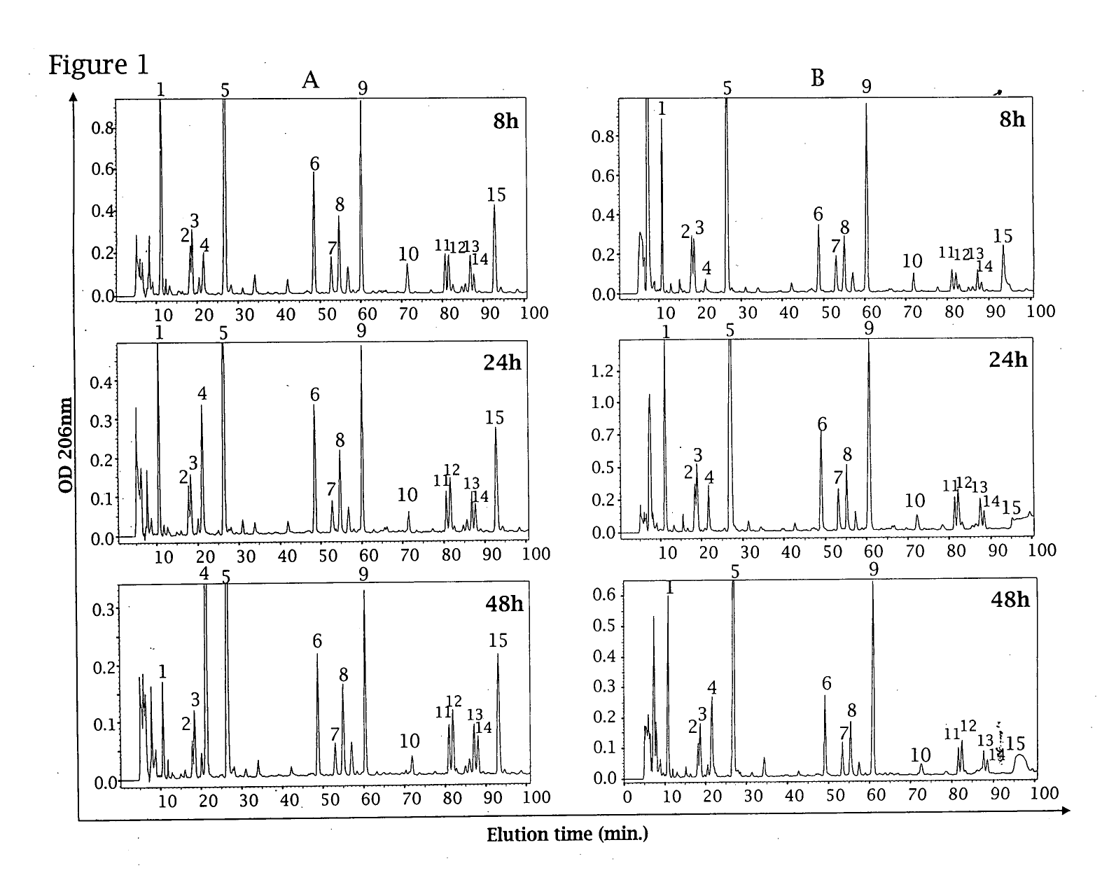

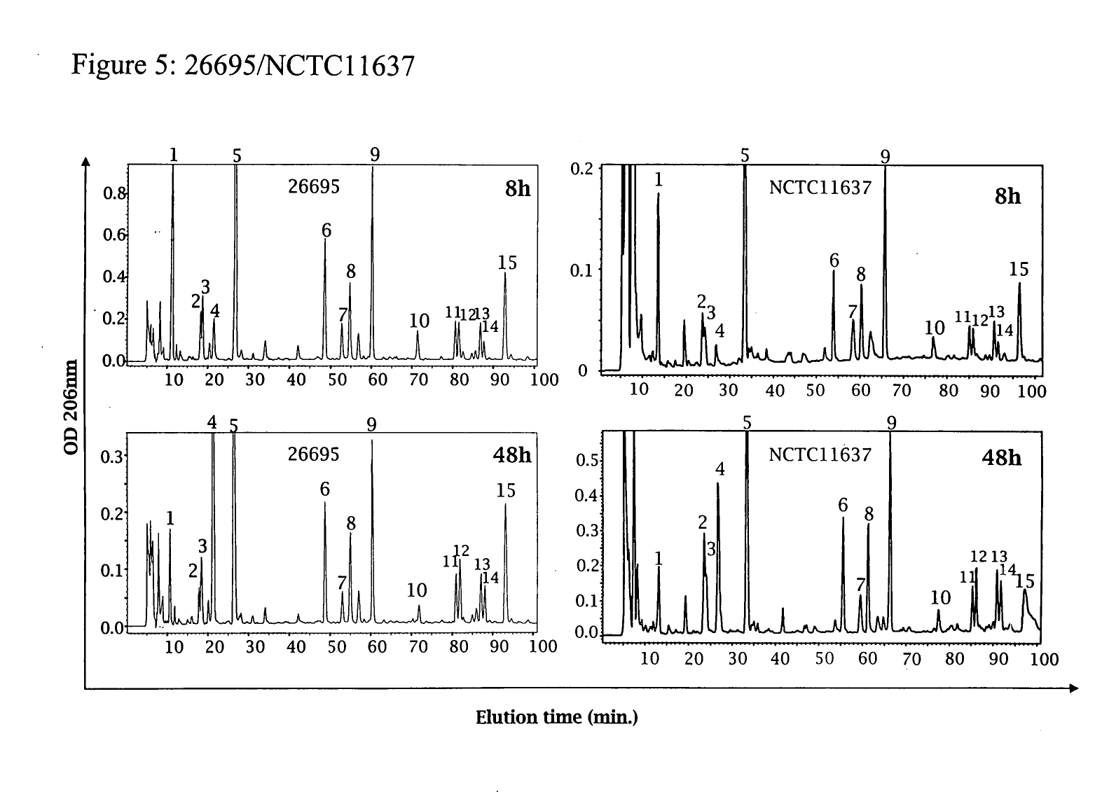

The inventors purified and analyzed the PG from the sequenced strain 26695 and from the strain NCTC11637 used as a control. No major difference between chromatograms of the two strains were observed (FIG. 1 and FIG. 5). Muropeptides composition analysis of H. pylori PG showed an accumulation of the GM-dipeptide motif in strain 26695 during the stationary phase, as previously observed in strain NCTC11637 (FIG. 1, FIG. 5 and [23]). Interestingly, the accumulation of the GM-dipeptide (peak 4) coincided with a decrease of N-acetyl-D-glucosaminyl-β(1,4)-N-acetylmuramyl-L-Ala-γ-D-Glu-mesoDAP (GM-tripeptide; peak 1).

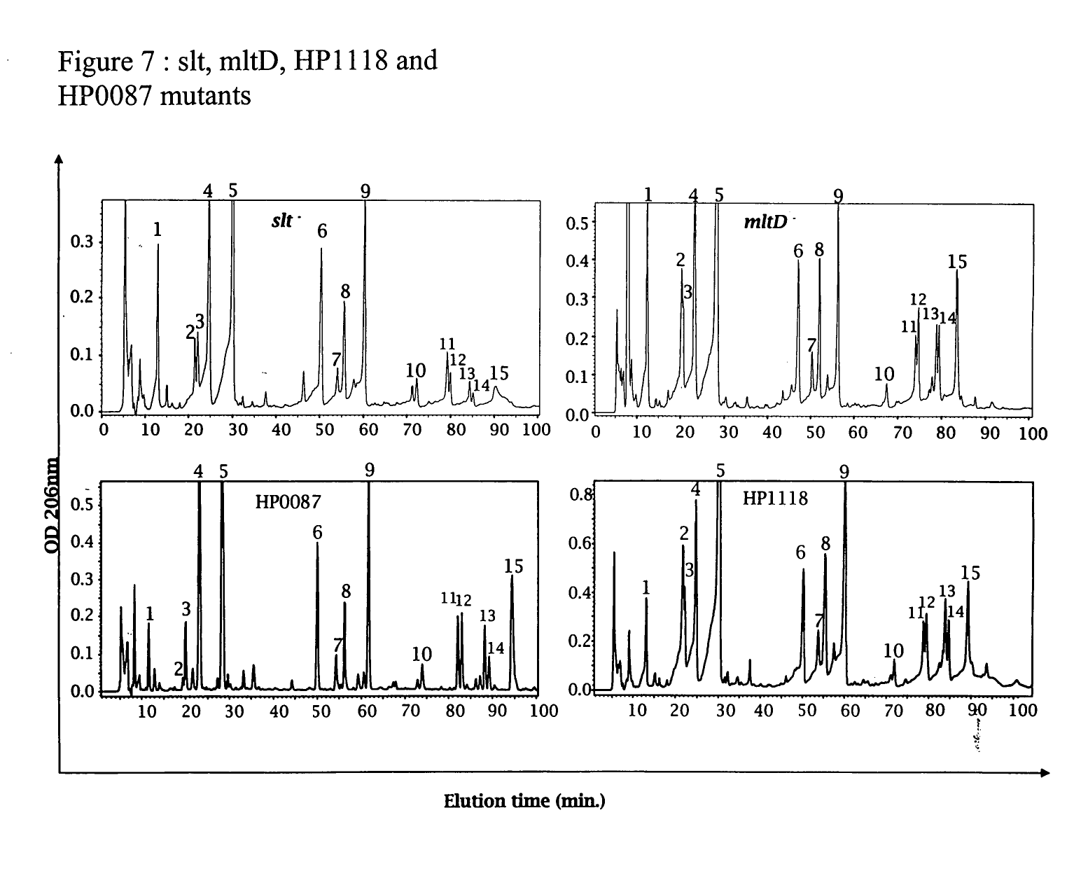

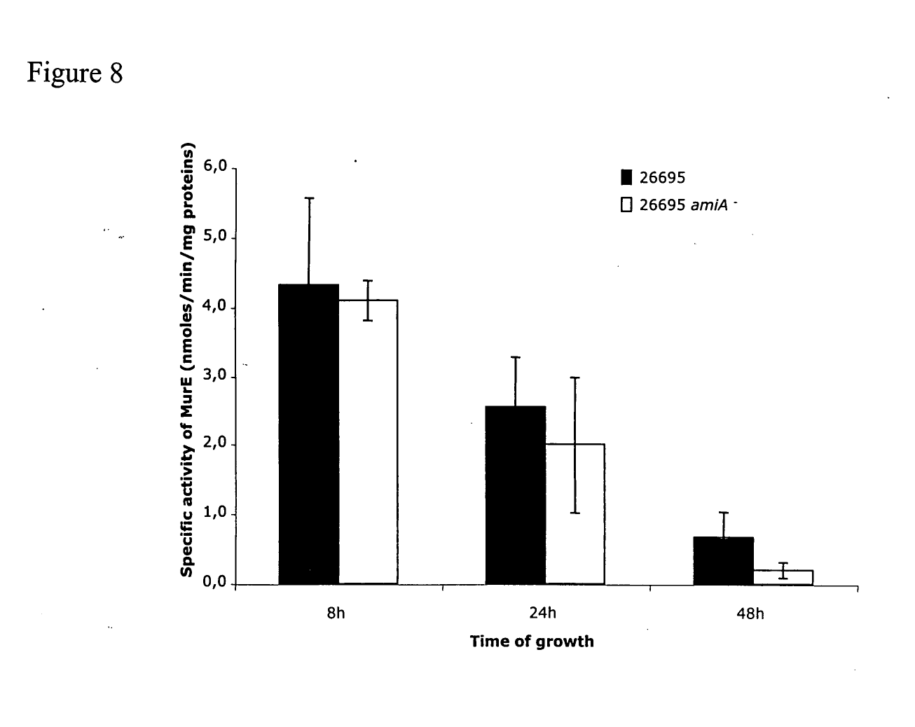

The inventors used a targeted approach to investigate the molecular mechanisms responsible for the accumulation of the GM-dipeptide motif (see supplementary material and FIG. 6). The inventors constructed mutants of hp0087 (encoding a putative peptidase), hp1118 (encoding a γ-GT), hp0645 (encoding the lytic transglycosylase Slt), hp1572 (encoding the lytic transglycosylase MltD) and hp0772 (encoding a putative N-acetyl-muramoyl-L-alanine amidase AmiA). Detailed information for each gene/protein is available on the PyloriGene 1 database (http://genolist.pasteur.fr/PyloriGene/genome.cgi). Only in the ami A mutant was the accumulation of GM-dipeptide (peak 4) impaired (FIG. 1 and FIG. 7). The PG of this mutant contained less of this motif at 8 h, 24 h and 48 h (about 1.9, 2.3 and 2.9 fold less, respectively), than the parental strain (FIG. 1). The amount of GM-tripeptide (peak 1) remained stable between exponential and stationary phase. The residual amount of GMdipeptide present in the PG of the ami A mutant is probably due to the decrease of MurE activity in stationary phase (FIG. 8).

### EXAMPLE 2

### Morphology of ami A Mutant

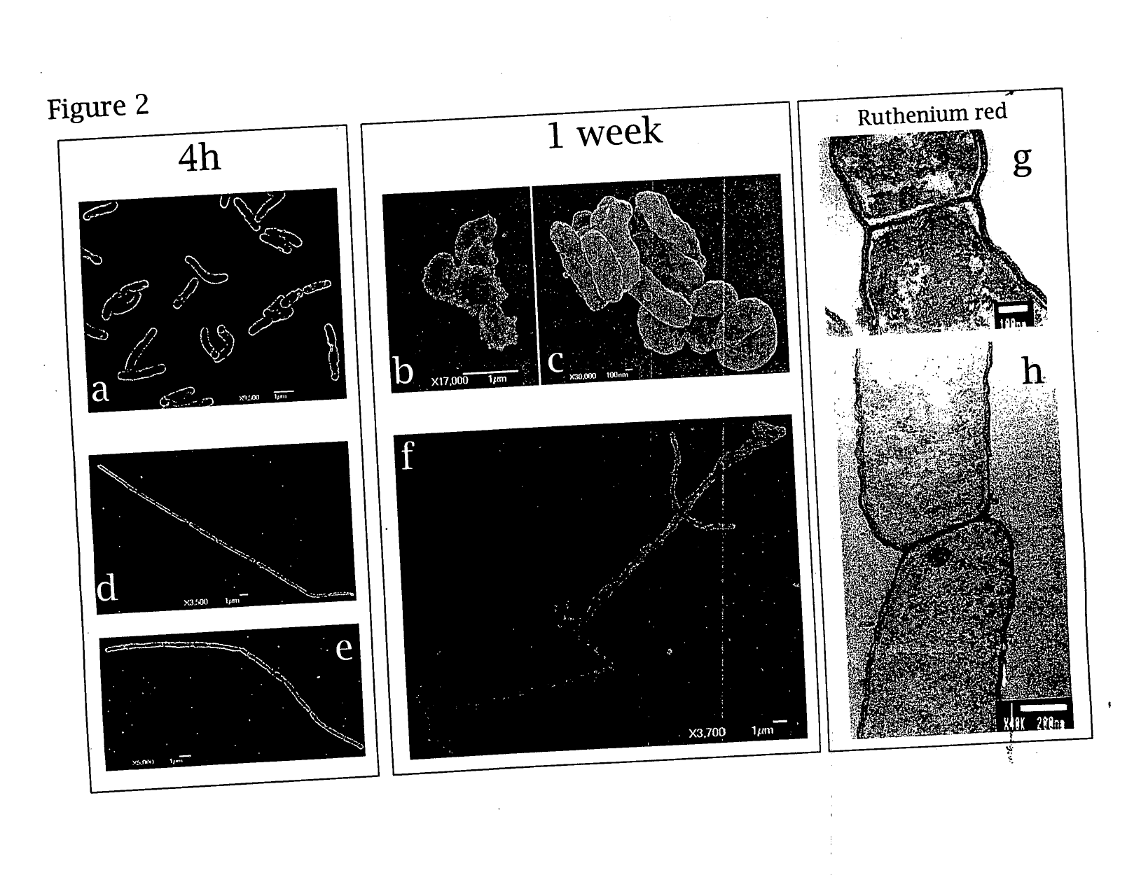

The inventors studied the morphology of the ami A mutant during the different growth stages by electron microscopy (EM): scanning EM and after ruthenium red staining (to visualize PG in the periplasmic space). Under negative contrast, the ami A mutant was observed as very long bacterial chains up to 30 bacteria per chain after 4 h of culture (FIG. 2, panels d and e) while the parental strain 26695 showed normal individual rod-shaped bacteria (FIG. 2, panel a). Sections were stained with ruthenium red revealing completely formed septums despite bacterial chaining (FIG. 2, panels g and h). Thus, cell daughter separation was defective in the ami A mutant. The parental strain, 26695, showed rod, U, donut and coccoid forms after 2 days, 1 week and one month of culture (FIG. 2, panels b, c and data not shown) while the ami A mutant remained in long chains of rod-shaped bacteria (FIG. 2, panel F). Far fewer ami A mutant cells were in coccoid forms after similar times of growth (Table 1). Therefore, the ami A mutant seems to be blocked both for the late steps of cell division and for the transition into coccoid forms.

TABLE 1
Quantification of the number of coccoid forms

| | | | | Number of |
|---|---|---|---|---|
| | | | | counted |
| Strain | days of growth | | % of coccoids^{a} | bacteria |
| 26695 | 1 | week | 55.79% | 699 |
| 26695 amiA⁻ | 1 | week | 6.26% | 405 |
| 26695 771::Tn3Km | 1 | week | 57.24% | 449 |
| 26695 + AMOX | 3-4 | h^{b} | 56.64% | 685 |
| 26695 amiA⁻+ AMOX | 3-4 | h^{b} | 32.60% | 1005 |

^{a}includes U and donut forms. For the amiA mutant, counts of bacteria correspond to individual bacteria that composed each chain
^{b}time of exposure to amoxicillin after 18 h of growth without antibiotic

### EXAMPLE 3

### Complementation of the ami A Mutant

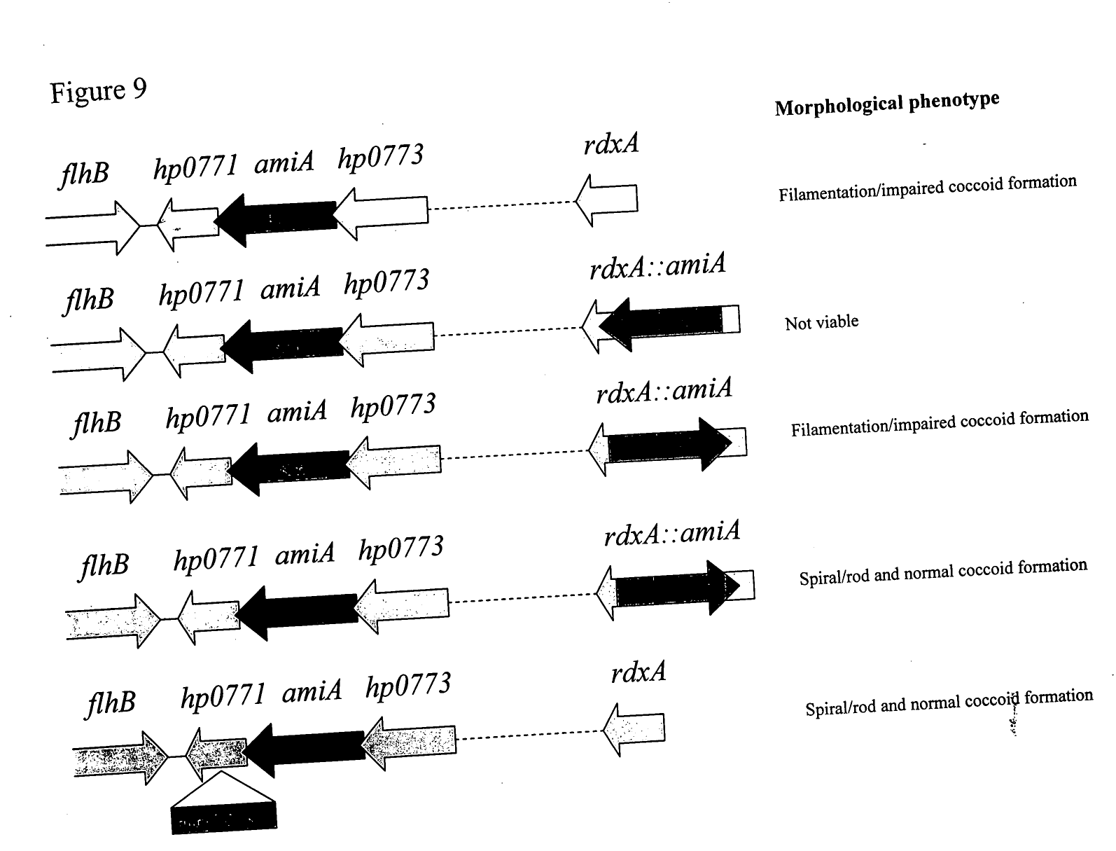

Next, the inventors tried to complement the phenotype by introducing a wild type ami A gene at a different locus, that of the rdxA gene (see FIG. 9). Disruption of the rarA gene confers metronidazole resistance to H. pylori [24]. However, the insertion of a copy of the Ami A gene into the rdxA gene in the same orientation was lethal for H. pylori. When the ami A gene was inserted into the rdxA gene in the opposite orientation, transformants were obtained. PCR analysis showed two populations of transformants: 1) one with ami A in rdxA and the wild-type ami A gene inactivated by the Km cassette (mtzRkmR mutants), and 2) mutants with ami A in rdxA and with the wild-type Ami A gene restored (mtzRkmS mutants). Only the second type of mutants (mtzRkmS) complemented the filamentation phenotype and restored the transition into coccoid forms. Hence, the observed phenotype could not be due to a secondary mutation. To eliminate the posibility of polar effects of the ami A mutant on the downstream gene, the inventors also constructed a mutant of the downstream gene, hp0771. The hp0771 mutant showed a normal bacillary form during the first day of culture, and the capacity to adopt the coccoid form. The inventors quantified the proportions of bacillary and coccoid forms (Table 1): the ami A mutant was the only strain impaired in the transition into coccoid forms.

### EXAMPLE 4

### The Effects of Amoxicillin

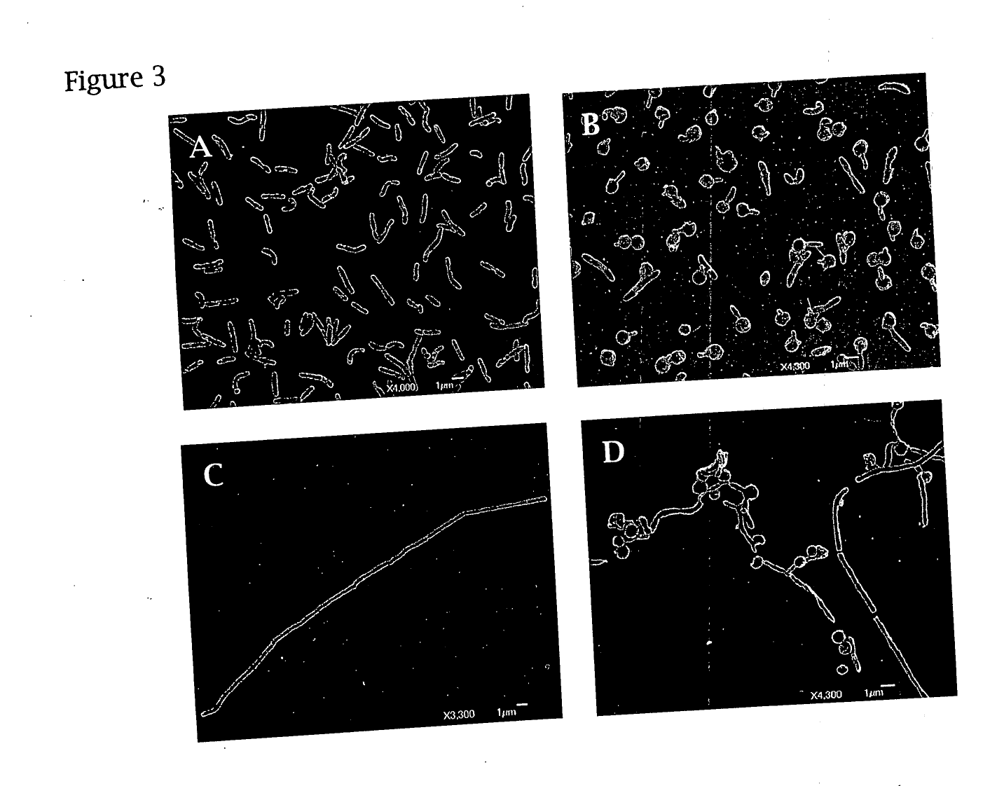

Some stress signals, including amoxicillin treatment, can induce the morphological transition into coccoid forms [4]. The inventors investigated the response of the ami A mutant to amoxicillin. First the inventors determined the MIC of amoxicillin: it was identical for the ami A mutant and the parental strain 26695 (0.06 μg/ml). After overnight culture, 10 μg/ml of amoxicillin was added to the media and after three hours of antibiotic treatment bacteria were observed by scanning electron microscopy. The ami A mutant formed chains of spherical bacteria (FIG. 3), of rod-shaped bacteria, and, most frequently, of both rod-shaped and spherical bacteria. Thus, the impaired morphological transition is not an artifact and does not result from steric hindrance of bacterial chain formation (see Table 1 for quantification). Thus, AmiA is required both for PG modifications and morphological transition.

### EXAMPLE 5

### Epithelial Cell Response to H. pylori PG and Coccoid Forms

Having demonstrated that the transition into coccoid forms is a controlled process by AmiA, the inventors investigated the biological role of the coccoid forms. The accumulation of the GM-dipeptide motif (FIG. 1; peak 4) correlated with the almost disappearance of the GM-tripeptide motif (FIG. 1; peak 1). These two muropeptides are the agonists of the Nod2 and Nod1 proteins, respectively [25]. Sensing of H. pylori PG by Nod1 is essential for the inflammatory response by gastric epithelial cells [26]. Therefore, the switch from a Nod1 agonist into a Nod2 agonist during coccoid formation could affect the ability of gastric epithelial cells to detect H. pylori and to develop an inflammatory response.

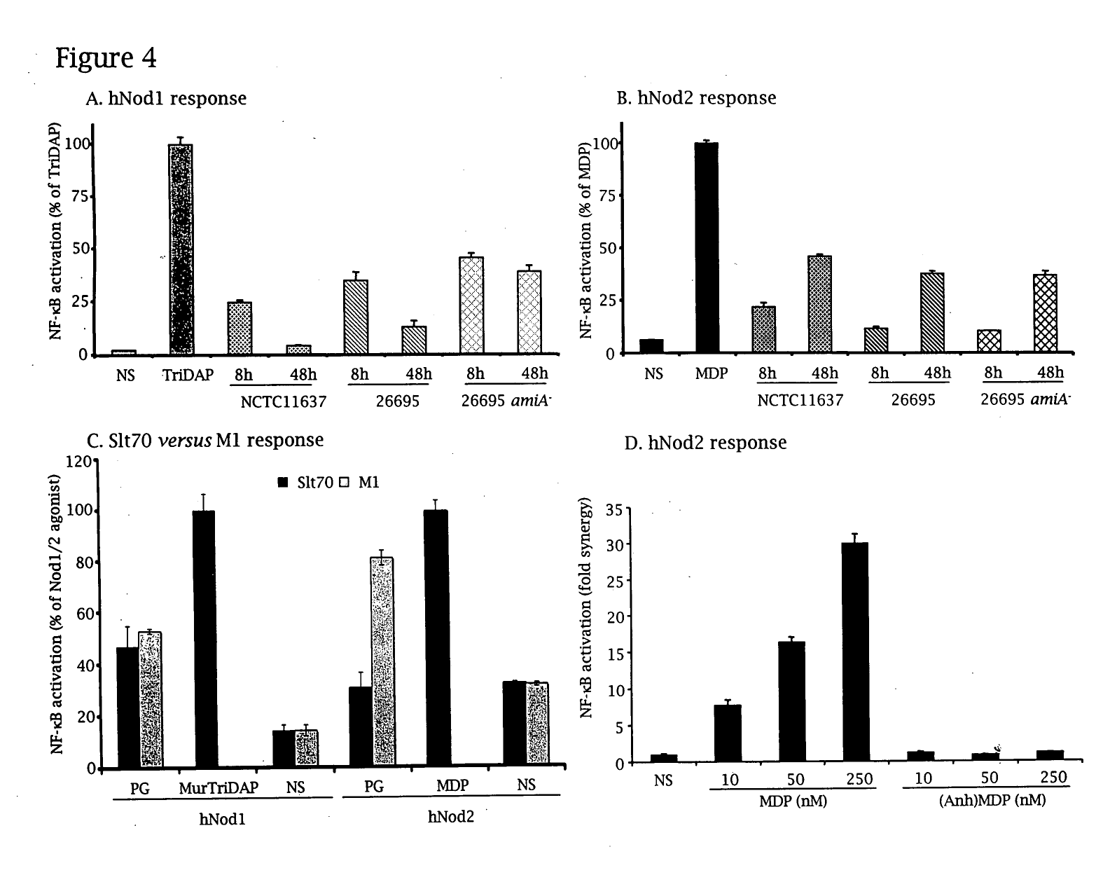

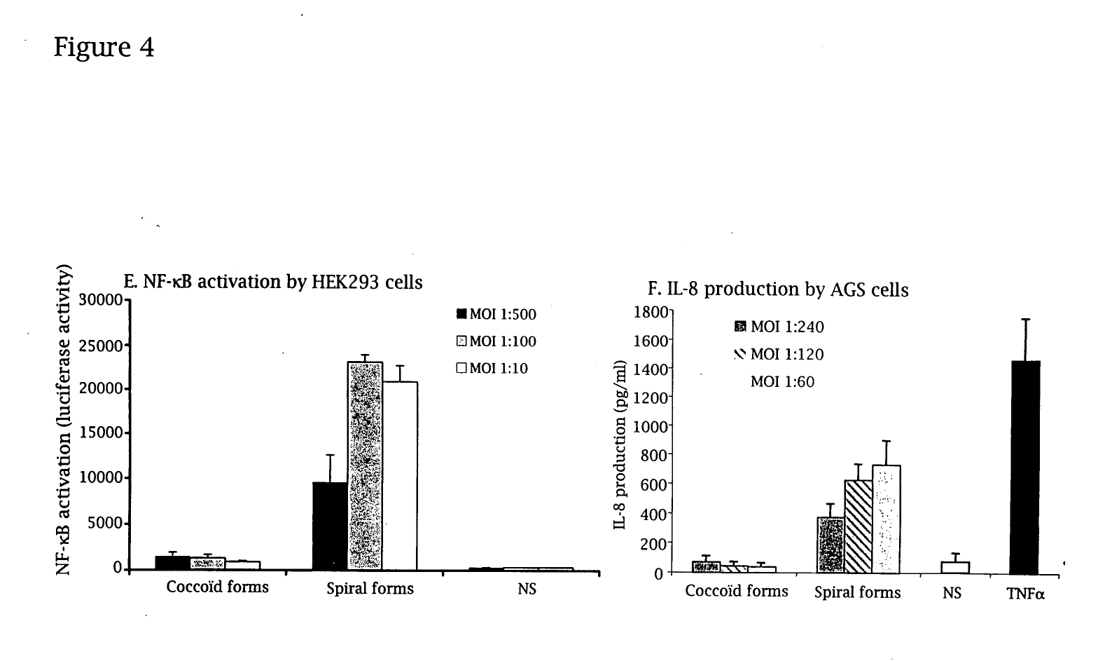

NF-κB activation in HEK293T cells via Nod1 and Nod2 was tested during stimulation with digested PG extracted from the ami A mutant and the parental strain after 8 h and 48 h of growth (FIG. 4A). Nod1 responses showed highest NF-κB activation with PG extracted after 8 h of growth and less activation with PG extracted at 48 h of growth, for both wild-type strains (26695 and NCTC 1637). Thus, the activation decreased with decreasing abundance of the GM-tripeptide in H. pylori PG. For the ami A mutant, Nod1 responses were the same when cells were stimulated with PG extracted after 8 h or 48 h of growth, consistent with the unchanging GM-tripeptide content of the PG. Inversely, Nod2 responses revealed a higher NF-κB activation with PG extracted after 48 h of growth with PG extracted after 8 h (FIG. 4B). These results suggest that spiral bacteria preferentially induce NF-κB via Nod1 and coccoid bacteria via Nod2.

However, Nod2 (as Nod1) senses muropeptides and not polymeric PG: the inventors therefore tested whether naturally occurring PG turnover products can stimulate Nod2. These products are mainly anhydromuropeptides generated by endogenous PG hydrolases named lytic transglycosylases. The inventors compared the Nod2-dependent activation of NF-κB by H. pylori PG digested by a muramidase (M1) and a lytic transglycosylases (Slt70 from E. coli).

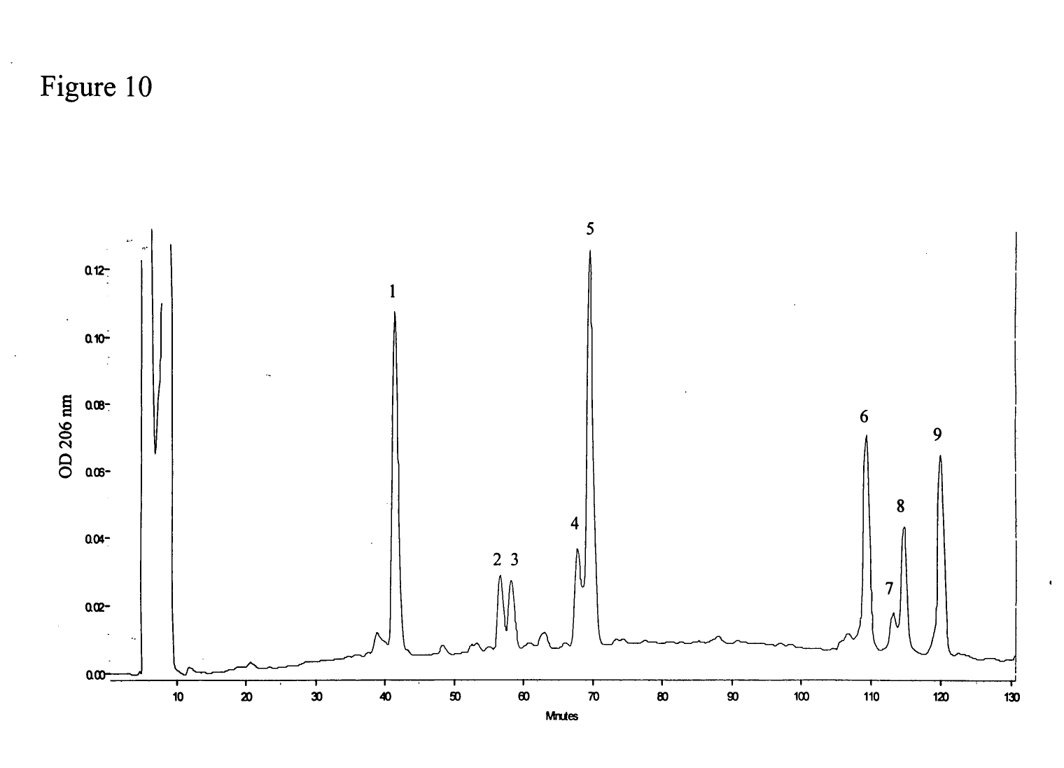

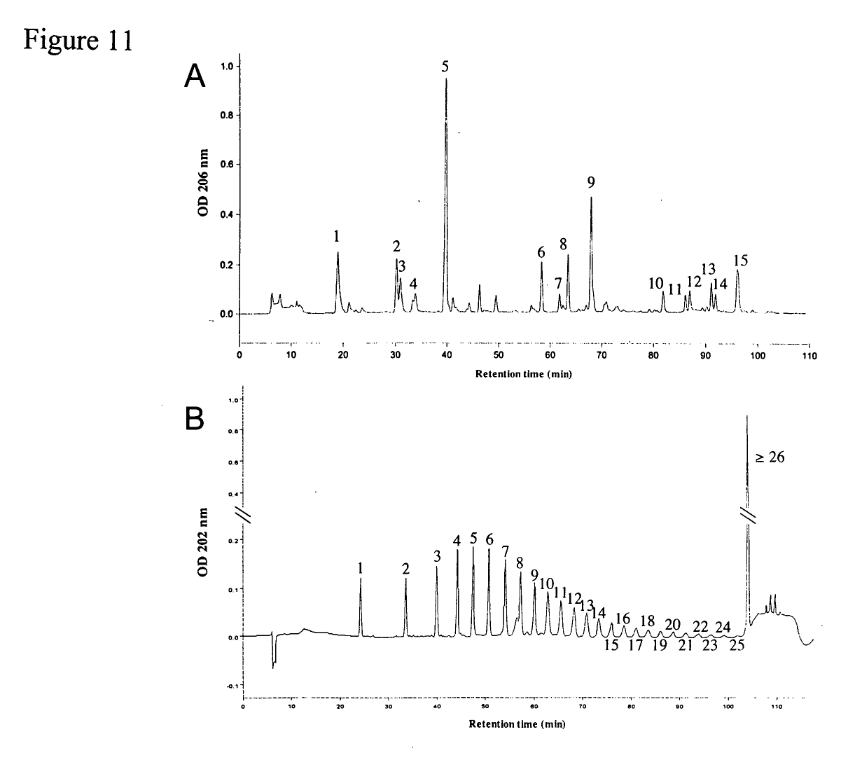

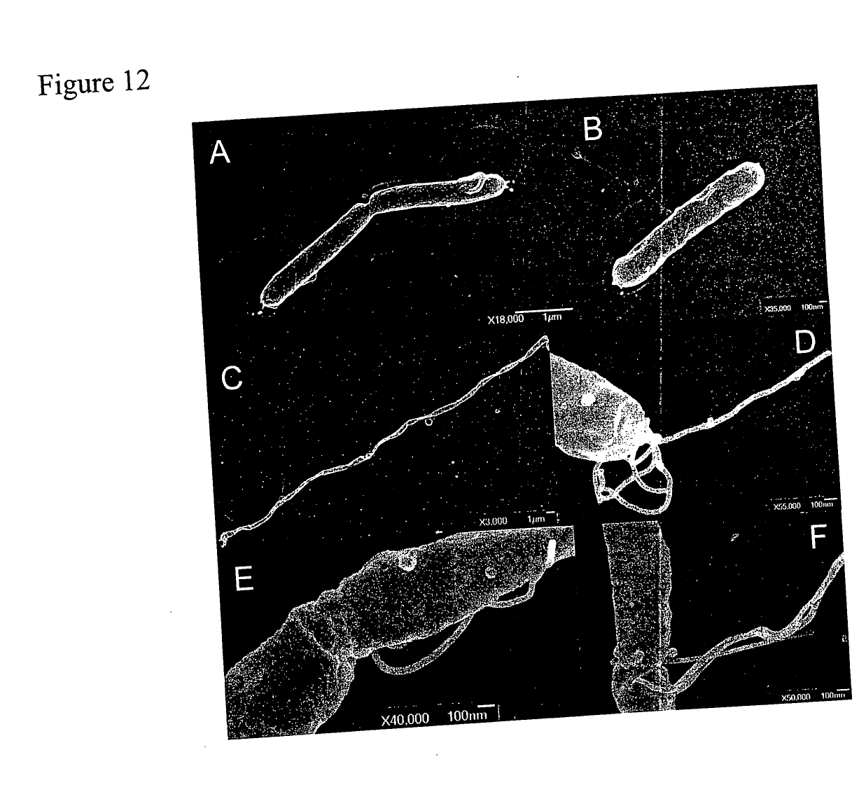

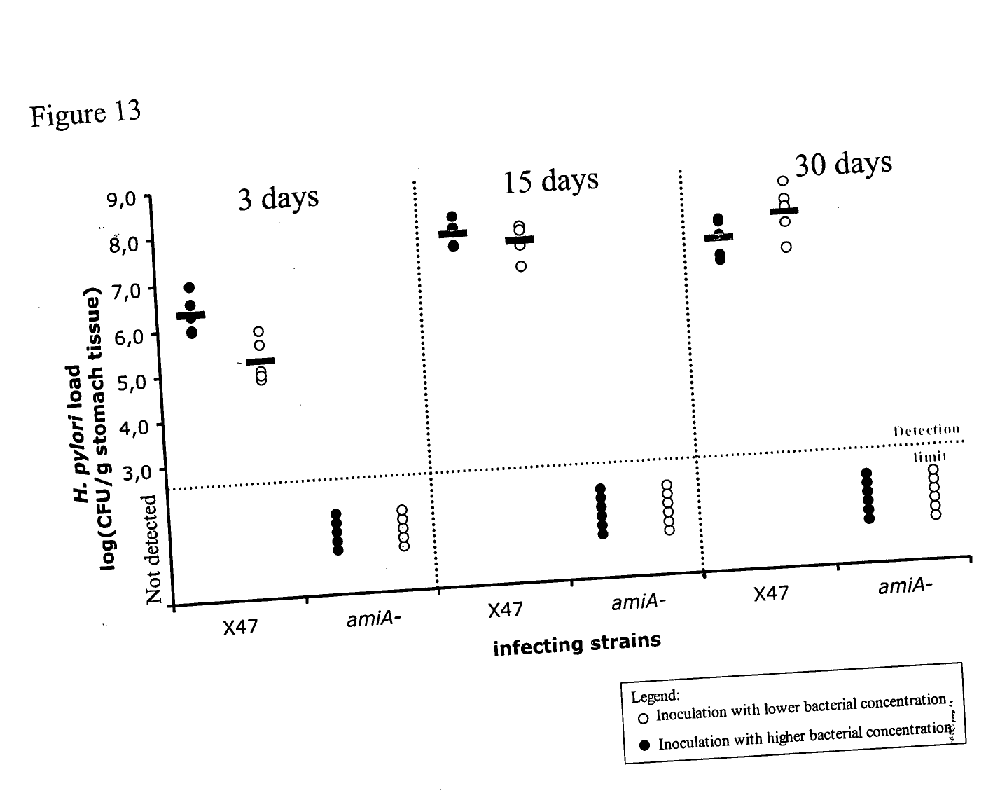

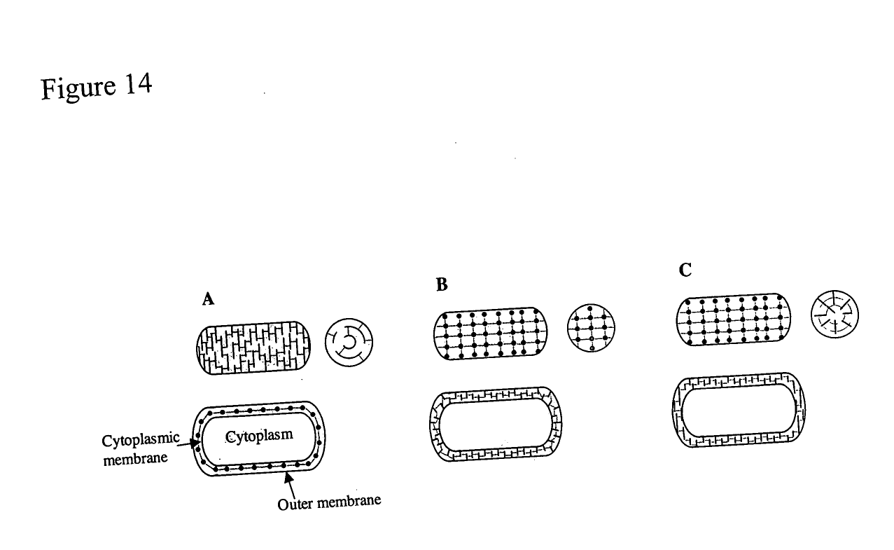

FIG. 10 shows the chromatogram of the Slt70 digested PG of H. pylori and the structural assignment of each anhydromuropeptide. Consistently with previous results (25), anhydromuropeptides were able to induce NF-κB in a Nod1-dependent manner (FIG. 4C). Surprisingly, anhydromuropeptides were unable to induce NF-κB in a Nod2-dependent manner. To further investigate the structural basis of Nod2 sensing, the inventors compared the Nod2-dependent activation of NF-κB by the GM-dipeptide to that by its anhydro derivative GanhM-dipeptide.

The GM-dipeptide motif produced by H. pylori was detected via Nod2 in a dose dependent manner. However, Nod2 did not sense the GanhM-dipeptide motif (FIG. 4D). The inventors concluded that PG turnover products are agonists of the Nod1 pathway (25), but are unable to induce the Nod2 pathway. Accordingly, rod-shaped H. pylori induced NF-κB in HEK293T cells and IL-8 production by gastric epithelial cells, but coccoid bacteria had no NF-κB or IL-8 stimulatory activities (FIGS. 4E and F). As epithelial cells do not respond to coccoid forms or to PG turnover products from coccoid forms, our study suggests that coccoid forms provide a route for immune escape for H. pylori.

Since the first observation of microbes, bacterial shape has been considered to be largely invariant and a characteristic feature of each species. It has therefore been used as a major taxonomic determinant. Nevertheless, several bacteria are know to change morphology during genetic developmental programs such as sporulation or asymmetric cell division. H. pylori undergoes morphological transition from spiral to coccoid. Previous attempts to identify specific markers or a dedicated genetic program involved in this morphological transition have been inconclusive [7,16-19]. Nevertheless, in 1999, Costa and colleagues correlated the morphological transition with a modification of H. pylori PG muropeptides composition [23], i.e. the accumulation of the GM-dipeptide motif.

The PG layer is a major determinant of bacterial cell shape, so the inventors felt that identifying the genetic determinants involved in the observed PG modification could help elucidate this morphological transition. There are several possible explanations for the accumulation of the GM-dipeptide motif (see supplementary material and supplementary FIG. 2). It could result from a defect in precursor synthesis in the cytoplasm due to: 1) a decrease of MurE activity blocking PG precursor synthesis at the addition of the meso-diaminopimelic acid (mesoDAP) step to the UDP-M-dipeptide, 2) insufficient mesoDAP to allow synthesis of precursors or, 3) the presence of a carboxy/endopeptidase, cleaving between the second and the third amino acid residue. This activity could be in either the cytoplasm (cleaving PG precursors with more than two amino acid residues in the stem peptide), or in the periplasm directly cleaving macromolecular PG.

The inventors also considered the potential roles of the annotated PG hydrolases Slt, MltD and AmiA in this process [27]. The supplementary material summarizes the various hypotheses and the data supporting or inconsistent with each of them. The inventors identified the ami A gene as necessary for the PG modification. The ami A mutant was impaired in the transition to coccoid forms. This is both the first identification of a genetic determinant required for the morphological transition of H. pylori, and directly implicates the PG layer in determining bacterial morphology. The inventors have found a putative PG hydrolase directly involved in maintenance of bacterial cell shape.

N-acetylmuramoyl-L-Alanine amidases contribute to the separation of daughter cells in Escherichia coli [28], but three genes encoding amidases had to be deleted from E. coli to observe a changed phenotype, whereas in H. pylori inactivation of a single gene was sufficient to observe a comparable filamentation phenotype.

Interestingly, the accumulation of GM-dipeptide motif (FIG. 1; peak 4) coincided with a proportional decrease of the GM-tripeptide motif (FIG. 1; peak 1). In the ami A mutant, the proportion of GM-tripeptide remained stable and the amounts of the GM-dipeptide were very low. No significant changes were observed for the other monomeric muropeptides. This is consistent with a periplasmic carboxy/endopeptidase activity that recognizes the γ-D-glutamyl-meso-diaminopimelic acid bond.

The AmiA protein is structured as a bimodular protein: a signal peptide followed by an N-terminal domain without homology to any sequences in the databases (1-177 amino acids (a.a.)), a linker peptide of variable length composed of KKEIP repeats (178-190 a.a.) and a C-terminal domain (191-440 a.a.) homologous to CwlU and CwlV, which are predicted to have a N-acetylmuramoyl-L-Alanine amidase activity [29]. PG amidases cleave the PG in the periplasm between the N-acetylmuramic acid residue and the first amino acid residue of the peptide moiety, L-Ala. However, the amidase activity of AmiA and its closest homologs have never been confirmed, so it is plausible that AmiA has a carboxy/endopeptidase activity.

Alternatively, AmiA might be bifunctional with an N-terminal carboxy/endopeptidase activity and a C-terminal amidase activity. It is also possible that AmiA has an amidase activity that is unable to cleave stem peptides with less than three amino acids residues such as the human serum amidase or PGRP-L [30]. This would lead to the elimination of stem peptides with three to five amino acid residues, and consequently the accumulation of GM-dipeptides.

The inventors showed that the morphological transition is regulated by AmiA. In its absence, the transition can be induced by treatment with amoxicillin, a β-lactam antibiotic. This suggests that other determinants involved in the morphological transition are pre-synthesized and potentially functional to lead the morphological transition. Exposure to amoxicillin bypasses the requirement for the AmiA protein, suggesting that one of the other determinants might be a penicillin-binding protein. Amoxicillin preferentially targets H. pylori PBP2 [4], a homolog of E. coli PBP2. A PBP2 conditional mutant of E. coli becomes spherical at non-permissive temperature [31] and consequently PBP2 is believed to drive lateral PG synthesis. Recruitment of S. aureus PBP2 to the site of PG synthesis requires the presence of its PG substrates [32]. The presence of AmiA results in an accumulation of GM-dipeptide. GM-dipeptide lacks the third amino acid, meso-diaminopimelate, which is crucial for PG polymerization by the PBPs.

Hence, it is possible that AmiA modifies the three dimensional structure of H. pylori PG, i.e. accumulation of GM-dipeptide preferentially on the lateral wall, favoring the synthesis of septal PG by PBP3 rather than lateral PG by PBP2. This would result in the inhibition of cell elongation and favor cell rounding, thus the formation of coccoid forms.

The role of the PG metabolism in the transition into coccoid forms suggests that this might be a regulated process rather than a random degeneration of H. pylori cells. Therefore, coccoid forms might be important in H. pylori physiology. Accordingly, Segal and colleagues showed that coccoid forms are able to translocate CagA, one of the major virulence factors and the only known effector protein of the H. pylori type IV secretion system, and induce cellular changes [33]. Coccoid forms express other virulence factors including the functional CagA.

The inventors show that coccoid forms modulate NF-κB activation. The morphological transition of H. pylori is accompanied by a decrease of the abundance of the GM-tripeptide motif, the Nod1 agonist, and this decrease minimizes the activation of NF-κB (via hNod1) in HEK293T cells and abolishes IL-8 induction in gastric epithelial cells. Thus, the coccoid forms might allow the bacteria to escape or modulate the host response and thereby to persist in the human stomach. This would be a previously undescribed mechanism for pathogens associated I with a chronic inflammatory response.

Nevertheless, coccoid forms may potentially stimulate epithelial cells via hNod2, in particular in an inflamed mucosa. Indeed, the hNod2 pathway can be induced by TNF-a and INF-γ in a NF-κB—dependent manner [34,35]. During a chronic infection of the gastric mucosa, coccoid forms of H. pylori would preferentially stimulate NF-κB via hNod2. However, hNod2 (as hNod1) senses muropeptides instead of polymeric PG. Muropeptides can be generated either by host lysozyme or by H. pylori endogenous lytic transglycosylases such as Slt. While lysozyme is abundant in paneth cells, it is almost absent from the mucus layer [36], where H. pylori preferentially resides [37].

Furthermore, as all Gram-negative bacteria, H. pylori is insensitive to lysozyme's activity. Muropeptides generated by the endogenous lytic transglycosylases such as GanhM-dipeptide (FIG. 4D) are not sensed by the hNod2 pathway. Hence, coccoid forms are unlikely to be seen by the host suggesting these could function as a mechanism of innate immune escape and modulation. Campylobacter jejuni also undergoes morphological transition into coccoid forms. C. jejuni usually causes acute gastroenteritis, but a recent study has associated long-term intestinal colonization of patients by C. jejuni with the onset of intestinal MALT lymphoma [38]. Possibly, coccoid forms of C. jejuni are similarly involved in establishing chronic infection.

In conclusion, the inventors report the ami A gene as the first genetic determinant of the transition from spiral bacteria into coccoid forms. This establishes AmiA as a practical target for identifying molecules which modulate the virulence of H. pylori, as well for studying how H. pylori regulates the transition from bacillary into coccoid forms and for investigating the physiological importance, in vitro and in vivo, of this particular bacterial form.

Bacteria, Cells and Growth Conditions.

Escherichia coli MC1061 [39] and DH5a were used as hosts for the construction and preparation of plasmids. They were cultivated in Luria Bertani solid or liquid media supplemented as appropriate with spectinomycin (100 μg.ml-1) or kanamycin (40 μg/ml) or both. H. pylori strain 26695 [40] was used to construct mutants. PG was extracted from strains 26695 and NCTC11637. H. pylori was grown microaerobically at 37° C. on blood agar plates or in liquid medium consisting of brain-heart infusion (BHI; Oxoid) with 0.2% β-cyclodextrin (Sigma) supplemented with antibiotic-antiftngic mix [41]. H. pylori mutants were selected on 20 μg/ml kanamycin. HEK293T cells were cultured in Dulbecco's modified Eagle's medium containing 10% fetal calf serum. Prior to transfection, HEK293T cells were seeded into 24-well plates at a density of 105 cells/ml as described previously [42].

Construction of Mutants and Complementation.

Genes were disrupted as described previously [43]. H. pylori mutants were constructed by allelic exchange after transformation with suicide plasmids or PCR products carrying the gene of interest interrupted by a non-polar cassette aphA-3 [43] or miniTn3-Km transposon and selected on kanamycin. PCRs were used to confirm that correct allelic exchange occurred. Gene constructions were sequenced to ensure sequence fidelity. All reagents, enzymes and kits were used according to manufacturers' recommendations. Midiprep (HiSpeed Plasmid Midi Kit) and DNA extraction kits (QIAamp DNA extraction kit) were purchased from QIAGEN.

The plasmid, pILL2000, was used to construct the ami A mutant pILL570 carrying ORF hp0772 (Ami A gene) was used as the template for an Expand High Fidelity PCR (Amersham) with oligonucleotides 772-1 (5′-gaugaugauggtaccaaggattttaacttcataagtc-3′ (SEQ ID NO: 7) in which the underlined sequence corresponds to a KpnI site) and 772-2 (5′-aucaucaucggatccaacacgcagcgattgatcgtctctaac-3′ (SEQ ID NO: 8) the underlined sequence corresponds to a BamHI site). PCR products were digested with BamHI (Amersham) and KpnI (Amersham) and ligated (T4 DNA ligase, Amersham) with the aphA-3 non polar cassette digested with the same endonucleases.

Complementation experiments were done by insertion of the Ami A in the rdxA locus, either in the same orientation or in the reverse orientation. The ami A mutant was used as a recipient for the suicide plasmid or PCR products for complementation.

Constructs were made as follows:

1) for the same orientation, the construct was made by three-time PCR [44]. Each of three fragments and final fragment used for transformation were obtained by Expand High Fidelity PCR. First, three fragments were obtained: i) 300-bp fragment corresponding to the 5′-end of rdxA obtained with oligonucleotides 954F (5′-atgaaatttttggatcaagaaaaaag-3′) (SEQ ID NO: 9) and CC772in954-1 (5′-CACAAGCACtacaaattaacctccattgaaatagatgtgcgctgc-3′ (SEQ ID NO: 10), capital letters corresponding to the sequence hybridizing with the 5′-end of the Ami A gene); ii) 1320-bp fragment corresponding to the Ami A gene obtained with oligonucleotides CCrbs772 (5′-gagggttaatttgtagtgcttgtg-3′) (SEQ ID NO: 11) and CC772stop (5′-ctaatcattcttgctgaagaaac-3′), (SEQ ID NO: 12) and iii) 300-bp fragment corresponding to the 3′-end of rdxA obtained with oligonucleotides 954Rev (5′-tcacaaccaagtaatcgcatcaac-3′) (SEQ ID NO: 13) and CC772in954-2 (5′-GTTTCTTCAGCAAGAATGATTAGtacctggagggaataatgcaatgctatatcgctgtgggg-3′ (SEQ ID NO: 14) capital letters corresponding to the sequence hybridizing with the 3′-end of the Ami A gene). The final PCR product was obtained by using a mixture of these three fragments as a template and oligonucleotides 954F and 954Rev.

2) for the reverse orientation, the pILL570-rdxA plasmid was used as the template for an Expand High Fidelity PCR (Amersham) with oligonucleotides 954-2KpnI (5′-cggggtacctacatgcaaaatctctatccg-3′ (SEQ ID NO: 15) in which the underlined sequence corresponds to a KpnI site) and 954-1BamHI (5′-cgcggatccgtgtggtaacaactcgctggg-3′ (SEQ ID NO: 16) the underlined sequence corresponds to a BamHI site). The Ami A gene was amplified using the following primers: 772-comp1-1Bis (5′-cggggatccgagggttaatttgtagtgcttgtgaggttagggg-3′ (SEQ ID NO: 17) in which the underlined sequence corresponds to a BamHI site) and 772-comp1-2Bis (5′-cgggtaccctaatcattcttgctgaaaaactatcaatgcc-3′ (SEQ ID NO: 18) the underlined sequence corresponds to a KpnI site). PCR products were digested with BamHI (Amersham) and KpnI (Amersham) and ligated (T4 DNA ligase, Amersham).

The hp0087 mutant was obtained following natural transformation of H. pylori with a construct made of three PCR products [44]. Each of three fragments and final fragment used for transformation were obtained by Expand High Fidelity PCR.

First, three fragments were obtained: i) 300-bp fragment corresponding to the 5′-end of HP0087 obtained with oligonucleotides 87-NotI (5′-ataagaatgcggccgcATGcgttattttcttgtagttttc-3′) (SEQ ID NO: 19) and 87-in1 (5′-GTTAGTCACCCGGGTACtgactttcatatctagccatgggg-3′ (SEQ ID NO:20), capital letters corresponding to the sequence hybridizing with the 5′-end of the aphA-3 gene); ii) 850-bp fragment corresponding to the aphA-3 cassette and iii) 300-bp fragment corresponding to the 3′-end of HP0087 obtained with oligonucleotides 87-EcoRI (5′-ggaattcaattcgcatttaaagggcttg-3′ (SEQ ID NO: 21) capital letters corresponding to the stop codon of HP0087) and 87-in2 (5′-TACCTGGAGGGAATAATGgactacatccttaaaaacgcc-3′ (SEQ ID NO: 22) capital letters corresponding to the sequence hybridizing with the 3′-end of the aphA-3 gene). The final PCR product was obtained by using a mixture of these three fragments as a template and oligonucleotides 87-NotI and 87-EcoRI.

γ-glutamyl transpeptidase (γ-GT; hp1118) and hp0771 mutants were obtained by gene interruption with miniTn3: there are no genes downstream from hp0771 and hp1118 with the same direction of transcription. The interruption was generated in E. coli DH5aby insertion of miniTn3 into plasmids carrying either hp0771 or hp1118 (Chantal Ecobichon et al.). Plasmids carrying the insertions were checked by PCR and used to transform H. pylori 26695. Mutants were validated by PCR analysis. The hp1118 mutant was also tested for the absence of γ-GT activity as previously described [45].

Peptidoglycan extraction and analysis. Liquid cultures of H. pylori parental strain and isogenic mutant strains were stopped after various times of growth and chilled in an ice-ethanol bath. The crude murein sacculus was immediately extracted in boiling sodium dodecyl sulphate (SDS; 4% final). Purification steps and high-pressure liquid chromatography (HPLC) analyses were as described previously [46]. Recombinant lytic transglycosylase Slt70 was purified as previously described [47]. M1− (Mutanolysin from Sigmna) or Slt70 -digested samples were analyzed by HPLC on a Hypersil ODS18 reverse-phase column (250 by 4.6 mm, 3 μm particle size) with a methanol (Fischer, HPLC grade) gradient from 0 to 15% in sodium phosphate buffer pH 4.3 to 5.0. Chromatograms were obtained by monitoring at 206 nm. Each peak was collected, desalted and identified by matrix-assisted laser desorption ionization mass spectrometry (MALDI-MS) as described previously [48].

Quantification of MurE activity. Bacteria were collected by centrifugation (3000 g, 20 min, 4° C.) from 400 ml of culture after 8 h, 24 h and 48 h of H. pylori growth. The bacterial pellets were washed with potassium phosphate buffer (20 mM, MgCl₂ 0.5 mM and 2-mercaptoethanol, pH 7.4), and resuspended in the same buffer. The cells were sonicated with a Branson sonifier at 20W per minute until the lysate was clear. The samples were dialyzed twice against the same buffer. MurE activity in these crude extracts was determined as described previously [49].

Electronic microscopy. Bacteria were washed with PBS (pH 7.4) and stained with ruthenium red or used directly for scanning electron microscopy. For ruthenium red staining, bacteria were prefixed with 2.5% glutaraldehyde, in 0.075% ruthenium red and 0.1M cacodylate buffer for 1 h. Samples were rinsed with 0.1M cacodylate buffer and post-fixed in 1% osmium tetraoxide in 0.1M cacodylate buffer for 2 h. They were washed in water three times then dehydrated in a series of ethanol concentrations. Finally, the samples were embedded in Spurr and ultrathin sections were made. Grids were viewed by transmission electron microscopy with a JEOL Jem 1010 microscope.

For scanning electron microscopy (SEM), samples were washed in PBS, prefixed in 2.5% glutaraldehyde in 0.1M cacodylate buffer for 30 minutes and then rinsed in 0.2M cacodylate buffer. After post-fixation in 1% osmium tetraoxide (in 0.2M cacodylate buffer), bacteria were dehydrated in a series of ethanol concentrations. Specimens were critical-point dried using carbon dioxide, then coated with gold and examined with a JEOL JSM-6700F SEM.

Minimal Inhibitory Concentration (MIC). To determine the MIC for amoxicillin, suspensions of H. pylori estimated to contain 108 bacteria/ml (OD600 nm of 0.1) were serially diluted and grown on plates containing various concentrations of amoxicillin. The MIC was defined as the amoxicillin concentration leading to a decrease of 3 log of CFU/ml as compared to growth without amoxicillin.

Expression Plasmids, Transient Transfections and NF-κB Activation Assays. The expression plasmid for FLAG-tagged hNod1 was from Gabriel Nuñez (University of Michigan Medical School, Ann Arbor, Mich.) and has been described previously [50]. The expression plasmid for hNod2 was from Gilles Thomas (Foundation Jean Dausset/CEPH, Paris, France). HEK293T cells were used for transfections as described previously [42]. Synergistic activation of NF-κB by PGs, muramyl peptides, and related compounds in cells over-expressing Nod1 or Nod2 was studied as described by Inohara et al. [51]. Briefly, HEK293T cells were transfected overnight with 10 ng of hNod1 or 30 ng of hNod2 plus 75 ng of Ig luciferase reporter plasmid. PG samples (0.1 μg/ml) were digested with 0.25 μg/μl mutanolysin. At the same time, 0.3 μg of PG preparations or 10 pmol of muramyl-peptides were added to the cell culture medium, and synergistic NF-κB-dependent luciferase activation was measured after 24 h of co-incubation. NF-κB-dependent luciferase assays were performed in duplicate, and data reported represent at least three independent experiments. Data was standardized with positive controls: M-dipeptide for hNod2 and M-tripeptide for hNod1. hNod1 and hNod2 were activated with H. pylori PG (0.3 μg/ml) digested with M1 or Slt70 as previously described [25].

Abbreviations Used in Section 1

γ-GT: gamma-glutamyltranspeptidase
G: N-acetyl-D-glucosamine
M: N-acetyl-muramic acid
(anh)M: N-acetyl-anhydromuramic acid
GM-dipeptide: N-acetyl-D-glucosminyl-β(1,4)-N-acetylmuramyl-L-Ala-D-Glu
GM-tripeptide: N-acetyl-D-glucosminyl-β(1,4)-N-acetylmuramyl-L-Ala-γ-D-Glu-mesoDAP
HPLC: high-pressure liquid chromatography
MALDI-MS: Matrix-assisted laser desorption ionization mass spectrometry
MIC: minimum inhibitory concentration
mesoDAP: meso-diaminopimelic acid
MOI: multiplicity of infection
mtz: metronidazole
km: kanamycin

### Section 2

The inventors have discovered that N-acetyl muramoyl-L-alanine amidase, or AmiA, H. pylori plays an important role in virulence of this organism in its host by participating in peptidoglycan metabolism, and cell daughter separation. By identifying compounds which inhibit or block these biological activities, it is possible to reduce the virulence and persistence of this pathogen.

The human gastric pathogen, Helicobacter pylori, is becoming increasingly resistant to most available antibiotics. Peptidoglycan metabolism is essential to eubacteria, hence, an excellent target for the development of new therapeutic strategies. However, little is known about peptidoglycan metabolism in H. pylori, in particular, the role of the peptidoglycan hydrolases.

The inventors have constructed an isogenic mutant of the Ami A gene encoding a N-acetylmuramoyl-L-alanyl amidase. The ami A mutant displayed long chains of unseparated cells, an impaired motility despite the presence of intact flagella and a tolerance to amoxicillin. Interestingly, the ami A mutant was impaired in colonizing the mouse stomach suggesting that AmiA is a valid target in H. pylori for the development of new antibiotics. Using reverse phase high-pressure liquid chromatography, the inventors analyzed the peptidoglycan muropeptide composition and glycan chain length distribution of strain 26695 and its ami A mutant. The analysis showed that H. pylori lacked muropeptides with a degree of cross-linking higher than dimeric muropeptides. The ami A mutant was also characterized by a decrease of muropeptides carrying 1,6-anhydro-N-acetylmuramic acid residues, which represent the ends of the glycan chains. This correlated with an increase of very long glycan strands in the ami A mutant. It is suggested that these longer glycan strands are trademarks of the division 1 site. Taken together the muropeptide composition, the glycan strand analysis and its inferred spatial distribution, the inventors provide evidence suggesting that between the different three-dimensional models of the peptidoglycan architecture, a modified version of the scaffold model accommodates best the ami A mutant PG structural analysis.

Helicobacter pylori is the etiological agent of duodenal and gastric ulcers, of gastric adenocarcinoma and mucosa associated lymphoid tissue lymphoma. It colonizes around half of the human population. Despite its medical importance, the inventors still have a fragmented knowledge of this human pathogen, in particular, regarding its physiology. The emergence of resistant strains to most available antibiotics active against H. pylori has stimulated the search for new therapeutic strategies against H. pylori.

The peptidoglycan (PG1 or murein) is an essential macromolecule that surrounds the cytoplasmic membrane and functions as an exoskeleton. PG is structurally composed of glycan strands of repeating disaccharide units of N-acetyl-D-glucosamine-β(1,4)-N-acetylmuramic acid (GM) cross-linked via short stem peptides creating one single huge molecule surrounding each bacterial cell. This exoskeleton is required to withstand turgor pressure, to maintain cell shape and cell division. Therefore, during cell growth, the PG layer has to be enlarged to accompany cell enlargement and daughter cells division and separation. Several models of the three dimensional organization of the PG layer have been proposed to fill with the experimental data of model bacteria among which the 3-for-1 and the scaffold models.

The essential nature of the peptidoglycan layer is evidenced by the wide success of antibiotics targeting bacterial cell wall synthesis such as β-lactams and glycopeptides. In this context, the inventors were interested to study PG metabolism in H. pylori for two reasons: 1) from the genome analysis, it appears that H. pylori has a restricted number of enzymes potentially involved in the PG assembly and maturation in the periplasmic space. There are only three PG synthetases, penicillin-binding proteins (PBPs) 1 to 3 and three putative PG hydrolases, two lytic transglycosylases, Slt and MltD, and one N-acetylmuramoyl-L-alanyl amidase, AmiA (Alm et al., 1999; Boneca et al., 2003; Tomb et al., 1997); 2) besides a previous characterization of the muropeptide composition of wild type H. pylori (Costa et al., 1999), no further work has been done on PG metabolism in H. pylori. Hence, a better understanding of PG metabolism in H. pylori could in the long-term lead to new therapeutic strategies.

The inventors addressed this issue by constructing and characterizing the isogenic ami A mutant and have shown that AmiA is required for cell daughter separation, correct motility and full virulence of H. pylori. Finally, the inventors have combined physiological data with muropeptide composition analysis and glycan strand length distribution by reverse-phase high-pressure liquid chromatography (HPLC) of the parental and ami A mutant and show that a modified version of the scaffold model is the one that best accounts the experimental data obtained for H. pylori.

Modifications in PG composition of ami A mutant. Analysis of the muropeptide composition of the wild type strain 26695 and the ami A mutant showed several modifications (FIG. 1A and Table 1).

TABLE 1
PG muropeptide composition of H. pylori 26695 and amiA mutant. Each peak numbering are illustrated
in FIG. 1A and corresponds to the nomenclature described by Costa and colleagues (Costa et al.,
1999). Each muropeptide structure was confirmed by MALDI-MS. Muropeptide abundance is expressed
as molar percentage and was calculated as desbrideb by Glauner (Glauner et al., 1998). Average
glycan chain length was calculated as described by Harz (Harz et al., 1990).

| | 26695 | | | 26695 amiA⁻ | | |
|---|---|---|---|---|---|---|
| Peaks | 8 h | 24 h | 48 h | 8 h | 24 h | 48 h |

Monomers

| | | | | | | | |
|---|---|---|---|---|---|---|---|
| 1 | GM-Tri | 16.8%_{±0.9%} | 13.7%_{±0.2%} | 4.9%_{±0.1%} | 13.5%_{±1.0%} | 17.7%_{±2.2%} | 14.6%_{±1.5%} |
| 2 | GM-Tetra | 5.2%_{±1.6%} | 3.7%_{±0.2%} | 2.6%_{±0.1%} | 6.7%_{±1.0%} | 4.7%_{±0.8%} | 3.8%_{±0.7%} |
| 3 | GM-Tetra-Gly | 4.0%_{±1.4%} | 4.8%_{±0.2%} | 5.0%_{±0.0%} | 5.0%_{±1.2%} | 4.0%_{±1.0%} | 5.6%_{±0.4%} |
| 4 | GM-Di | 3.3%_{±1.0%} | 10.9%_{±0.2%} | 23.3%_{±0.4%} | 1.7%_{±0.7%} | 3.8%_{±1.0%} | 10.3%_{±1.0%} |
| 5 | GM-Penta | 37.6%_{±2.4%} | 31.9%_{±0.7%} | 31.6%_{±0.3%} | 41.2%_{±1.6%} | 39.8%_{±2.6%} | 38.6%_{±3.6%} |

Dimers

| | | | | | | | |
|---|---|---|---|---|---|---|---|
| 6 | GM-Tetra-Tri-GM | 5.1%_{±0.5%} | 5.6%_{±0.0%} | 4.6%_{±0.1%} | 3.5%_{±0.4%} | 4.5%_{±0.4%} | 4.1%_{±0.4%} |
| 7 | GM-Tetra-TetraGly-GM | 2.0%_{±0.5%} | 1.7%_{±0.1%} | 1.4%_{±0.2%} | 1.9%_{±0.4%} | 2.0%_{±0.5%} | 2.0%_{±0.2%} |
| 8 | GM-Tetra-Tetra-GM | 3.6%_{±0.2%} | 3.8%_{±0.1%} | 3.7%_{±0.4%} | 3.0%_{±0.3%} | 2.8%_{±0.5%} | 3.1%_{±0.5%} |
| 9 | GM-Tetra-Penta-GM | 9.4%_{±0.5%} | 8.4%_{±0.0%} | 7.2%_{±0.0%} | 11.3%_{±1.0%} | 10.0%_{±1.25%} | 11.2%_{±0.5%} |

Anhydromuropeptides

| | | | | | | | |
|---|---|---|---|---|---|---|---|
| 10 | anhGM-Penta | 2.6%_{±0.5%} | 2.3%_{±0.2%} | 1.8%_{±0.0%} | 2.7%_{±0.6%} | 1.6%_{±0.6%} | 1.4%_{±0.5%} |
| 11 | anhGM-Tetra-Tri-GM | 1.9%_{±0.4%} | 1.8%_{±0.0%} | 1.9%_{±0.0%} | 1.5%_{±0.3%} | 2.0%_{±0.4%} | 1.6%_{±0.5%} |
| 12 | anhGM-Tetra-Tri-GM | 1.4%_{±0.4%} | 2.6%_{±0.1%} | 2.7%_{±0.0%} | 1.2%_{±0.2%} | 1.7%_{±0.4%} | 1.5%_{±0.2%} |
| 13 | anhGM-Tetra-Tetra-GM | 1.4%_{±0.5%} | 1.8%_{±0.1%} | 2.0%_{±0.0%} | 1.3%_{±0.2%} | 1.2%_{±0.2%} | 1.2%_{±0.0%} |
| 14 | anhGM-Tetra-Tetra-GM | 1.0%_{±0.3%} | 1.4%_{±0.1%} | 1.5%_{±0.1%} | 0.6%_{±0.2%} | 0.7%_{±0.2%} | 0.6%_{±0.1%} |
| 15 | anhGM-Tetra-Penta-GM | 4.8%_{±0.1%} | 5.7%_{±0.3%} | 5.9%_{±0.2%} | 5.0%_{±1.2%} | 3.3%_{±2.0%} | 0.4%_{±0.5%} |
| 4 | Dipeptides | 3.3%_{±1.0%} | 10.9%_{±0.2%} | 23.3%_{±0.4%} | 1.7%_{±0.7%} | 3.8%_{±1.0%} | 10.3%_{±1.0%} |
| 1, 6, | Tripeptides | 25.2%_{±1.4%} | 23.6%_{±0.3%} | 14.1%_{±0.1%} | 19.7%_{±0.7%} | 25.9%_{±1.9%} | 21.8%_{±1.5%} |
| 11, 12 | | | | | | | |
| 2, 3, | Tetrapeptides | 41.8%_{±1.2%} | 43.3%_{±0.6%} | 40.6%_{±1.1%} | 40.9%_{±2.7%} | 37.7%_{±2.3%} | 34.4%_{±0.5%} |
| 7-9, | | | | | | | |
| 11-15 | | | | | | | |
| 3, 7 | Tetrapeptides-Glycin | 6.0%_{±1.1%} | 6.5%_{±0.1%} | 6.4%_{±0.2%} | 6.9%_{±1.0%} | 6.1%_{±1.1%} | 7.6%_{±0.6%} |
| 5, 9, | Pentapeptides | 54.4%_{±1.5%} | 48.3%_{±0.7%} | 46.4%_{±0.5%} | 60.2%_{±1.7%} | 54.7%_{±1.6%} | 51.6%_{±3.5%} |
| 10, 15 | | | | | | | |
| 1-5, | Monomers | 69.4%_{±1.2%} | 67.4%_{±0.5%} | 69.2%_{±0.5%} | 70.7%_{±3.0%} | 71.7%_{±2.2%} | 74.3%_{±0.0%} |
| 10 | | | | | | | |
| 6-9, | Dimers | 30.6%_{±1.2%} | 32.6%_{±0.5%} | 30.8%_{±0.5%} | 29.3%_{±3.0%} | 28.3%_{±2.2%} | 25.7%_{±0.0%} |
| 11, 15 | | | | | | | |
| 10-15 | Anhydromuropeptides | 13.0%_{±0.9%} | 15.5%_{±0.6%} | 15.8%_{±0.2%} | 12.2%_{±1.9%} | 10.6%_{±2.3%} | 6.7%_{±0.2%} |
| | Average glycan | 10.2_{±0.8} | 8.5_{±0.3} | 8.3_{±0.1} | 10.7_{±2.1} | 12.5_{±2.3} | 18.7_{±0.7} |
| | chains length | | | | | | |

In exponentially growing bacteria, the inventors observed an increase in proportion in muropeptides carrying pentapeptides and a decrease of the ones carrying tripeptides or dipeptides. The most striking difference concerned the proportion of the N-acetyl-D-glucosaminyl-β(1,4)-N-acetylmuramyl-L-Ala-D-Glu (GM-dipeptide) motif at different times of the growth curve. While the wild type accumulated this motif in stationary phase (48 h), the ami A mutant did it to a much lower extent.

Otherwise, another strong modification of the PG composition at 48 h of growth was the decrease of anhydro-muropeptides in the PG of ami A mutant (Table 1), (about 2.3 fold lower than the parental strain 26695). Anhydro88 muropeptides consist of muropeptides carrying an N-acetyl-anhydromuramic acid residue (anhM), which is a signature for the end of glycan chains in Gram-negative bacteria. So, the relative amounts of anhydro-muropeptides can be correlated to the length of glycan chain. This difference was mainly due to the decrease of dimeric GanhM-tetrapeptide-pentapeptide-MG and the monomeric GanhM-penta.

During exponential growth, the ami A mutant had glycan chains of an average of 10.7 disaccharide units comparable to the wild type strain (10.2). However, in stationary phase, the average increased to 18.7 disaccharide repeating units, compared to 8.3 disaccharide repeating units for the wild type. Consequently, the ami A mutant seemed to have longer glycan chains than the parental strain 26695 in stationary phase. Inversely, the major dimer GM-tetra-penta-GM increased in the ami A mutant (11.2% versus 7.2%). However, overall the percentage of dimers was lower in the ami A mutant, particularly, in stationary phase (25.7% versus 30.8%). Interestingly, no new muropeptides including highly cross-linked muropeptides such as trimers or tetramers were identified either in the wild type or the ami A mutant.

Glycan chain length distribution. Since a major feature of the ami A mutant was the decrease of anhydro-muropeptides, the inventors analyzed the glycan chain length of the wild type and the ami A mutant at 8 h of growth (FIG. 1B and Table 2). Generation of glycan chains was obtained using the human serum amidase, which has a specificity for stem peptides with 3 or more amino acids but is unable to cleave the GM-dipeptide (Wang et al., 2003). Hence, the inventors were unable to compare the glycan chain length at 24 h and 48 h because the wild type strain accumulates the GM-dipeptide motif. As expected, glycan strand analysis did not require prior amino sugar reduction for HPLC separation of the different peaks confirming that the glycan strands end exclusively by 1,6-anhydro-N-acetylmuramic acid residues (FIG. 1B).

Interestingly, the ami A mutant showed a shift towards shorter glyvcan chains (Table 2). While the proportion of short glycan chains (=5 disaccharide repeating units) increased, glycan chains between 6 and 16 disaccharide repeating units decreased. However, the overall average glycan chain length of strands up to 25 disaccharide units decreased moderately from 5.3 to 4.9 disaccharide repeating units. Inversely, the proportion of very long glycan chains (=26 disaccharide repeating units) increased substantially from 17.5% to 22.5%.

TABLE 2
Glycan strand, length distribution analysis of H. pylori. Each glycan
strand species corresponds to the different peaks in FIG. 1B. The
nomenclature of each peak refers to the number of disaccharide repeating
units per glycan strand specie. The UV percentage takes into account
the total glycan strand UV absorbing material separated by HPLC (FIG.
1B). The molar percentage can be calculated for the 25 first peaks
by dividing the UV percentage by the number of disaccharide units
of each glycan species. The final glycan strand peak is a mixture
of different species for which the relative proportions are unknown.
Therefore, we estimated the average glycan strand length of the very
long chains to have a gross estimate of their molar proportion. To
determine the average chain length for glycans ≧ 26 disaccharide
units, we used the following formula: =(average length − UV
%[peaks 1-5]*average length[peaks 1-25]/UV %[peaks ≧ 26].
The average glycan strand length was calculated in Table 1 (10.2
and 10.7 for 26695 and 26695 amiA, respectively). The average length
for the glycan chains up to 25 disaccharide units were calculated as
described by Harz (13). We obtained an average of 5.3 and 4.9 for
26695 and 26695 amiA, respectively. The average length of glycans
with more than 26 disaccharide units is 33.4 and 30.7 disaccharide
repeating units for 26695 and 26695 amiA, respectively.

| disaccharide | UV % | | Molar % | |
|---|---|---|---|---|
| units | 26695 | 26695 amiA⁻ | 26695 | 26695 amiA⁻ |
| 1 | 3.12% | 3.62% | 3.12% | 3.62% |
| 2 | 3.44% | 3.80% | 1.72% | 1.90% |
| 3 | 4.63% | 5.46% | 1.54% | 1.82% |
| 4 | 6.36% | 6.73% | 1.59% | 1.68% |
| 5 | 7.20% | 7.32% | 1.44% | 1.46% |
| 6 | 7.49% | 6.97% | 1.25% | 1.16% |
| 7 | 7.73% | 6.27% | 1.10% | 0.90% |
| 8 | 6.37% | 5.42% | 0.80% | 0.68% |
| 9 | 5.87% | 4.70% | 0.65% | 0.52% |
| 10 | 5.41% | 4.99% | 0.54% | 0.50% |
| 11 | 4.22% | 3.17% | 0.38% | 0.29% |
| 12 | 3.70% | 2.82% | 0.31% | 0.24% |
| 13 | 3.10% | 2.68% | 0.24% | 0.21% |
| 14 | 2.67% | 2.37% | 0.19% | 0.17% |
| 15 | 2.16% | 1.91% | 0.14% | 0.13% |
| 16 | 1.73% | 1.50% | 0.11% | 0.09% |
| 17 | 1.48% | 1.37% | 0.09% | 0.08% |
| 18 | 1.26% | 1.24% | 0.07% | 0.07% |
| 19 | 1.04% | 1.12% | 0.05% | 0.06% |
| 20 | 0.91% | 1.05% | 0.05% | 0.05% |
| 21 | 0.79% | 0.94% | 0.04% | 0.04% |
| 22 | 0.67% | 0.82% | 0.03% | 0.04% |
| 23 | 0.58% | 0.72% | 0.03% | 0.03% |
| 24 | 0.45% | 0.60% | 0.02% | 0.03% |
| 25 | 0.19% | 0.00% | 0.01% | 0.00% |
| 1 to 5 | 24.75% | 26.93% | 9.41% | 10.49% |
| 6 to 25 | 57.82% | 50.63 | 6.09% | 5.28% |
| ≧26 | 17.43% | 22.44% | 0.52% | 0.73% |

Susceptibility to different antibiotics. As shown above, AmiA has a major role in the structure and composition of H. pylori PG. Thus, the inventors were interested in characterizing the resistance phenotype of the ami A mutant to several classes of antibiotics, in particular, β-lactam antibiotics.

The MIC and MBC values of amoxicillin were both 0.06 μg/ml for the parental strain 26695 122 (Table 3). So, the MBC/MIC ratio was 1 for strain 26695. The amiA mutant showed MIC value 0.06 μg/ml of amoxicillin identical to the parental strain. But MBC value for the mutant was superior than the maximum amoxicillin concentration tested (32 μg/ml). Therefore, the ami A mutant showed a MCB/MIC ratio >256 and could be considered as tolerant to amoxicillin. The complemented ami A mutant had similar MIC value than parental strain and the mutant. It had a MBC/MIC ratio of 2, similar to that of the parental one. These results showed that AmiA is needed for the bactericidal activity of amoxicillin.

Finally, the inventors tested the resistance phenotype to several other classes of antibiotics. The ami A mutant had the same pattern of antibiotic resistance as the parental strain (Table 3). This indicates that contrary to E. coli, inactivation of the single amidase of H. pylori does not affect the overall outer membrane architecture but rather only PG metabolism.

TABLE 3
Minimum bactericidal and inhibitor concentration (MBC and
MIC) of amoxicillin and other antibiotics for H. pylori
strain 26695, mutant amiA and the complemented mutant.

| | MIC | MCB | |
|---|---|---|---|
| | (μg/ml of | (μg/ml of | Ratio |
| Strain | amoxicillin) | amoxicillin) | MBC/MIC |
| 26695 | 0.06 | 0.06 | 1 |
| 26695 amiA− | 0.06 | >32 | >256 |
| 26695 amiA− | 0.125 | 0.250 | 2 |
| complemented | | | |

| | | |
|---|---|---|
| MIC (μg/ml) | 26695 | 26695 amiA |

| | | | | |
|---|---|---|---|---|
| Streptomycin | 1 | μg/ml | 1 | μg/ml |
| Bacitracin | >1000 | μg/ml | >1000 | μg/ml |
| Nalidixic acid | 30 | μg/ml | 30 | μg/ml |
| Metronidazole | 1 | μg/ml | 1 | μg/ml |
| Vancomycin | >1000 | μg/ml | >1000 | μg/ml |
| Trimethroprime | >100 | μg/ml | >100 | μg/ml |

Morphological analysis of the ami A mutant. Next, the inventors were interested in analyzing the general morphological phenotype of the ami A mutant since amidases have been implicated in cell daughter separation both in Gram-positive and Gram-negative bacteria. As observed for several other bacteria, the inactivation of the ami A gene resulted in a chaining phenotype (FIG. 2). Also, H. pylori is known for undergoing a morphological transition from spiral to coccoid form during entry in stationary phase. The inventors observed that associated with the chaining phenotype, the ami A mutant failed to undergo morphological transition.

Since the sequenced strain 26695 lacks flagella, the inventors also constructed several independent clones in other H. pylori backgrounds. Interestingly, when the Ami A gene was inactivated in strains that were motile such as X47-2AL (FIGS. 2C to F) and B128, the mutants were still able to synthesize at the poles (FIG. 2D) and some division sites intact flagella (FIGS. 2E and F)). Although some bacterial chains were motile under the optical microscope, the vast majority were not. Using a soft agar mobility assay, all the ami Aindependent mutant clones were unable to migrate from the site of inoculation in contrast to the wild type strain.

Colonization of mice stomachs. Since the ami A mutant had two major cell morphological defects, impaired cell daughter separation and motility, the inventors investigated the impact on the ami A inactivation on H. pylori capability to colonize mice stomachs. The inventors infected C57/BL6J mice with two parental and fully motile strains, X47-2AL (FIG. 3) and B 128 (data not shown), and their isogenic ami A mutants. The inventors then analyzed their ability to colonize the mouse gastric mucosa at different time points (3, 15 and 30 days of infection; see FIG. 3). Note that the infections were done with an even mixture of three independent clones of ami Amutants in each background. Clearly, the ami A mutant was unable to colonize the stomach of C57/BL6J mice under any conditions tested, indicating that the AmiA protein is required for efficient colonization of the stomach.

In H. pylori the single ami A gene fulfills the same role in cell daughter separation as that played collectively by the three amidases of E. coli. The ami A mutants constructed in different genetic backgrounds (26695, X47-2AL and B128) present long bacterial chains with up to 30-40 bacteria in which the division site was completely formed but without cell daughter separation. This observation underlines the major role played by amidases in cell daughter separation both in Gram-negative and Gram-positive bacteria. Interestingly, despite impaired cell daughter separation, ami A mutants derived from parental flagellated H. pylori were still capable to assemble intact flagella at the site of cell division. The inventors can thus assume that these new division sites are fully functional for flagella assembly, although these flagella appeared to be paralyzed. Therefore, whichever are the structural modifications of the PG layer at the new cell poles in the ami A mutant, these do not seem to hinder flagella assembly but only flagella function.

As it is well known that fully motile bacteria are essential for H. pylori colonization of the stomach (Ottemann and Lowenthal, 2002), our observation that the ami Adeficient strains do i not colonize is consistent with their “paralyzed” phenotype. These results make AmiA an attractive new target against H. pylori. H. pylori is one of the few bacteria, against which, a specific antibiotherapy that does not affect the commensal flora is recommended due to its high world prevalence. Interfering with normal H. pylori AmiA function would fits such a strategy. The H. pylori AmiA is phylogenically distante from Gram-negative amidases and resembles most CwlU and CwlV from Paenibacillus polymyxa and an amidase from Deinococcus radiodurans. Hence, specific inhibitors of AmiA function would probably not affect amidases from other commensal bacteria. Amidases have also been involved in the mechanism of β-lactam induced lysis and death.

Interestingly, the H. pylori ami A mutant became tolerant to amoxicillin similarly to the lytA mutant of S. pneumoniae (Tomasz et al., 1970). The ratio of MBC over MIC was higher than 256, while complementation of the ami A mutant restored a wild type ratio (ratio of 2). As for other bacteria, in H. pylori, AmiA plays a major role in the mechanism of β-lactam induced death. However, the inventors have shown that β-lactam antibiotics do not induce lysis of H. pylori (Chaput et al., submitted) but only cell rounding (or coccoid formation). Exposure of the ami A mutant to 100 times its MIC to amoxicillin still induced coccoid formation (Chaput et al.). Hence, the cell rounding can be dissociated from cell death since the ami A mutant is tolerant to amoxicillin.

The mechanism of cell death in wild type bacteria and tolerance of the ami A mutant remain a mystery. However, the inventors can reasonably assume that it is directly related to the three dimensional modifications of the PG layer that occurs at the division site. In E. coli, the purified PG of the amidase mutants is resistant to lysozyme treatment (Costa et al., 1999).

The ami A mutant of H. pylori seems to have longer glycan chains. Therefore, such PG is less prone to degradation by the endogenous lytic transglycosylases. Despite inhibition of PG synthesis by amoxicillin, a localized resistance to the action of endogenous hydrolases at the poles could account for the observed tolerance of the ami A mutant.

The remaining phenotypes diverged substantially from the E. coli example. One of the major observations supporting the 3-for-1 model is the presence in the PG of E. coli (and a variety of other Gram-negative bacteria) of trimeric muropeptides (Glauner et al., 1988; Quintela et al., 1995). Interestingly, H. pylori appeared to be an exception since it lacked trimeric muropeptides or muropeptides with a higher degree of cross-linking (Table 1 and (Costa et al., 1999)). Since inactivation of the three amidases of E. coli resulted in an increase of trimeric and tetrameric muropeptides and consequently an increase in the degree of cross209 linking, the inventors reasoned that the ami A mutant of H. pylori should exhibit the presence of such structures in the PG layer of H. pylori. Surprisingly, the inventors did not observe any trace of trimeric muropeptides.

Other major differences in muropeptide composition between the parental and ami A strain were observed when bacteria entered stationary phase (24 h and 48 h of growth). The wild type strain showed an increase of the anhydro-muropeptides from exponential to stationary phase. These muropeptides represent the glycan chains ends (Harz et al., 1990), and their proportion gives an estimate of the average length of the glycan chains. The same is valid for H. pylori as shown by the glycan chain analysis by HPLC. Exponentially growing and stationary phase bacteria had glycan chains with an average of 10.2 and 8.3 disaccharides units, respectively.

The ami A had the same average as the wild type during exponential growth (10.7 disaccharide repeating units). However, in stationary phase the average increased drastically to 18.7 disaccharide repeating units. Furthermore, the degree of cross-linking in the ami A mutant decreased. This is in sharp contrast with the triple amidase mutant of E. coli, for which not only the degree of cross-linking was increased but where the average glycan chain length decreased (Heidrich et al., 2001).

Intuitively, these changes in cross-linking and glycan chain length seem logical. When the degree of cross-linking decreases one expects to have a looser network. Therefore, increasing the glycan chain length increases the chances of two distinct glycan chains to be connected by a cross-bridge.

These changes in glycan strand structure were confirmed by a more precise analysis of the glycan chain length distribution by HPLC. Comparison of the HPLC profiles of the wild type and the ami A mutant (FIG. 1) revealed that the overall distribution of the different glycan species was distinct. The ami A mutant showed enrichment in very short and very long glycans, while glycans with intermediate length decrease (Table 2).

From the microscopy observation of the ami A mutant, the only morphological distinct difference concerned the impaired cell daughter division. This observation taken together with a net increase of the glycan chain length in stationary phase for the ami A mutant when the bacterial chains increased the most, strongly suggests that the septum PG is composed primarily of very long glycan chains while the lateral wall PG would be of very short ones. Interestingly, an E. coli ftsZ84 thermosensitive mutant grown at permissive temperature fails to initiate cell division and filaments. Consequently, the ftsZ84 mutant synthesizes exclusively lateral wall PG. Glycan chain length distribution of the ftsZ84 mutant showed an enrichment of very short glycan chains and a substantial decrease of very long chains again (Ishidate et al., 1998). Unfortunately, the inventors could not corroborate the phenotype in H. pylori since ftsZ (hp0979) is an essential gene. However, a preferred distribution of short glycans at the septum and very long chains at the lateral wall would be incompatible with the amount of PG per cell given the very low molar abundance of long glycan chains (Table 2).

Several models of the three dimensional organization of the PG layer have been proposed to fit with the experimental data (Vollmer and Holtje, 2004). Along the proposed models, the 3 for-1 model considers that the glycan chains are parallel to the cytoplasmic membrane (FIG. 4A). The average length of a H. pylori cell is 1.5×0.5 μm. Using the same calculations as Vollmer and Holtje (Vollmer and Holtje, 2004), to cover the periplasm with a single PG layer (total surface of 3.14 μm²) would require 6.04×10⁵ muropeptide molecules; a muropeptide covers 5.2 nm² (3.14×10⁶/5.2). From the muropeptide composition of H. pylori (Table 1), the average muropeptide has a 4 amino acids stem peptide (MW 939.39 Da). Thus, the inventors can estimate the weight of PG per cell to be 6.04×10⁵×939.39/6.022×10²³=0.942×10-15 g. Thus, 10⁹ bacteria would theoretically yield 0.942 μg of PG, which is compatible with our experimental data for H. pylori (1 μg per 1.6×10⁹ cells; see (Travassos et al., 2004). As for the scaffold model, it considers glycan strands to be perpendicular to the cytoplasmic membrane (FIG. 4B). Since a disaccharide unit is 1.03 nm long, the average glycan length of exponentially growing bacteria (10.5 disaccharide units) is incompatible with the thickness of the periplasmic space (5-6 nm). Furthermore, if the inventors calculate the amount of PG necessary to cover one cell considering that each glycan chain covers an area of 27 nm², the inventors realize as for E. coli that the scaffold model is again incompatible with H. pylori bacterial life (1.90 μg of PG per 10⁹ cells).

However, as discussed above, the glycan chain length distribution is not uniform and from the analysis of our ami A mutant and data from the E. coli ftsZ84 mutant, the lateral wall PG would be enriched in short glycan chains. Based on the molar proportion of the different glycan chain species separated by HPLC, which constitute 83% of the total UV absorbing material, the average length of the glycan chains for these species is 5.3 disaccharide units (average length 5.5 nm). This is compatible with the thickness of the periplasmic space (around 6 nm). The inventors consider that the remaining 17% of very long glycan chains are present exclusively at division sites and poles. Given the average length and radius of H. pylori (1.5 μm×0.5 μm), the inventors can estimate that the average lateral wall surface per cell is 2.35 μm². As calculated by Vollmer and Holtje (Vollmer and Holtje, 2004), the surface that a glycan chain can cover equals 27 nm². Therefore, H. pylori would require 2.35×10⁶*5.3/27=4.63×10⁵ muropeptides to covert its lateral wall. Again considering that in average the stem peptides are composed of 4 amino acids, the inventors estimate that to cover the lateral wall, 10⁹ cells would require 0.72 μg of PG. To calculate the amount of PG required to cover the division sites and the poles, the inventors consider that the glycan chains are synthesized perpendicular to the constricting membrane (see FIG. 4C). Hence at the poles and division sites the glycan chains remain parallel to the cytoplasmic membrane instead of perpendicular as at the lateral wall. The inventors estimated that the surface to cover division sites and poles consisted of half-spheres. This approximation over-estimates the surface to cover since division sites resemble more the side of a cylinder. But since the proportion of division sites or poles is unknown, the inventors considered that all the bacteria were separated. Each bacterium has two poles and therefore, the surface to cover corresponds to 0.785 μm² for H. pylori. Since the glycan strands are parallel to the membrane the minimal subunit is the extended muropeptide as for the 3-for-1 model (5.22 nm). Thus, 0.785×10⁶/5.2=1.51×10⁵ molecules are require per cell, which corresponds to 0.236 μg per 10⁹ bacteria. Therefore, 10⁹ cells would have yield 0.96 μg of PG, which is compatible with the experimental data (1 μg per 1.6×10⁹ cells). If the inventors consider the scaffold model imposing a particular location of the short versus long glycans, the inventors can cover an entire H. pylori cell.

Finally, both models are compatible with the experimental data, except for the complete absence of trimeric muropeptides in H. pylori. However, the inventors cannot exclude that H. pylori might generate transiently trimeric muropeptides, which are rapidly processed to dimers and monomers. From the muropeptide analysis, H. pylori incorporates preferably intact pentapeptides.

Nevertheless, tetrapeptide moieties are found either as monomers or dimers despite the absence of known carboxy-and/or endopeptidases. The presence of such tetrapeptides could be generated by a new family of carboxy-and/or endopeptidases explaining the absence of trimeric muropeptides. However, given the small genome, the restricted number of PBPs and putative PG hydrolases, it is unlikely for H. pylori to have developed unique strategies to assemble its PG layer compared to other Gram-negative bacteria. Alternatively, the known high-molecular weight PBPs (PBP1, 2 and 3) could function both as transpeptidases and carboxy/endopeptidases. Clearly, the absence of trimeric muropeptides is in disagreement with the 3-for-1 model while these structures are not required for the scaffold model. Therefore, the analysis of the ami A mutant of H. pylori favors our <<modified >> scaffold model over the 3-for-1 model.

Experimental Procedures

### EXAMPLE 6

### Bacteria, Cells and Growth Conditions

Escherichia coli MC1061 (Casadaban and Cohen, 1980) and DH5a were used as hosts for the construction and preparation of plasmids. They were cultivated in Luria Bertani solid or liquid media supplemented as appropriate with spectinomycin (100 μg/ml) or kanamycin (40 μg/ml) or both. H. pylori strain 26695 (Tomb et al., 1997), X47-2AL (Londono-Arcila et al., 2002) and B128 (Israel et al., 2001) were used to construct mutants. PG was extracted from strain 26695. H. pylori was grown microaerobically at 37° C. on blood agar plates or in liquid medium consisting of brain-heart infusion (BHI; Oxoid) with 0.2% β-cyclodextrin (Sigma) supplemented with antibiotic antiflngic mix (Bury-Mone et al., 2004). H. pylori mutants were selected on 20 μg/ml kanamycin.

### EXAMPLE 7

### Construction of Mutants and Complementation

Genes were disrupted as described previously (Skouloubris et al., 1998). H. pylori mutants were constructed by allelic exchange after transformation with suicide plasmids or PCR products carrying the gene of interest interrupted by a non-polar cassette aphA-3 (Skouloubris et al., 1998) and selected on kanamycin. PCRs were used to confirm that correct allelic exchange occurred. Gene constructions were sequenced to ensure sequence fidelity. All reagents, enzymes and kits were used according to manufacturers' recommendations. Midiprep (HiSpeed Plasmid Midi Kit) and DNA extraction kits (QIAamp DNA extraction kit) were purchased from QIAGEN. The plasmid, pILL2000, was used to construct the ami A mutant pILL570 carrying ORF hp0772 (Ami A gene) was used as the template for an Expand High Fidelity PCR (Amersham) with oligonucleotides 772-1 5′-GAUGAUGAUGGTACCAAGGATTTTAACTTCATAAGTC-3′ (SEQ ID NO: 23) in which the underlined sequence corresponds to a KpnI site) and 772-2 (5′-AUCAUCAUCGGATCCAACACGCAGCGATTGATCGTCTCTAAC-3′ (SEQ ID NO: 24) the underlined sequence corresponds to a BamHI site). PCR products were digested with BamHI (Amersham) and KpnI (Amersham) and ligated (T4 DNA ligase, Amersham) with the aphA-3 non-polar cassette digested with the same endonucleases. For complementation, the promorterless wild type Ami A gene was introduced in the rdxA gene carried by plasmid pILL570. The plasmid was used as the template for an Expand High Fidelity PCR (Amersham) with oligonucleotides 954-2KpnI (5′-CGGGGTACCTACATGCAAAATCTCTATCCG-3′ (SEQ ID NO: 25) in which the underlined sequence corresponds to a KpnI site) and 954-1BamHI (5′-CGCGGATCCGTGTGGTAACAACTCGCTGGG-3′ (SEQ ID NO: 26) the underlined sequence corresponds to a BamHI site). The Ami A gene was amplified using the following primers: 772-comp1-1Bis (5′-CGGGGATCCGAGGGTTAATTTGTAGTGCTTGTGAGGTTAGGGG-3′ (SEQ ID NO: 27) in which the underlined sequence corresponds to a BamHI site) and 772-comp1-2Bis (5′-CGGGTACCCTAATCATTCTTGCTGAAAAACTATCAATGCC-3′ (SEQ ID NO: 28) the underlined sequence corresponds to a KpnI site). PCR products were digested with BamHI (Amersham) and KpnI (Amersham) and ligated (T4 DNA ligase, Amersham).

### EXAMPLE 8

### Peptidoglycan Extraction and Analysis

Liquid cultures of H. pylori parental strain and isogenic mutant strains were stopped after various times of growth and chilled in an ice351 ethanol bath. The crude murein sacculus was immediately extracted in boiling sodium dodecyl sulphate (4% final). Purification steps and HPLC analyses were done as described previously (Glauner, 1988). Mutanolysin (Sigma) digested samples were analyzed by HPLC on a Hypersil ODS18 reverse-phase column (250 by 4.6 mm, 3 μm particle size) with a methanol (Fischer, HPLC grade) gradient from 0 to 15% in sodium phosphate buffer pH 4.3 to 5.0. Chromatograms were obtained by monitoring at 206 nm. Each peak was collected, desalted and identified by matrix-assisted laser desorption ionization mass spectrometry (MALDI-MS) as described previously (Antignac et al., 2003). Glycan chain analysis was done as previously described (Boneca et al., 2000; Harz et al., 1990). Briefly, H. pylori PG was digested with purified human serum amidase kindly provided by Waldemar Volhmer. The digestion was done in 50 mM Tris-HCl pH 7.9, 5 mM MgCl₂, 0.02% NaN₃. Soluble material was first purified on a MonoS (HR5/5) column (Amersham Pharmacia) using a 10 mM sodium phosphate buffer pH 2. Glycans eluted with the flow-through and were collected. Free peptides were eluted by one step using 10 mM sodium phosphate buffer pH 2, 1 M NaCl. The runs performed at room temperature using a flow of 1 ml/min. Purified glycans were analyzed by reverse phase HPLC using a 5 μm

Nucleosil 300 C18 column (250×4.6 mm) at 50° C. A convex gradient from 0 to 10.5% acetonitrile (−4 curve of the Shimadzu CLASS-VP software) in 100 mM sodium phosphate buffer pH 2 was used over 90 minutes at a flow rate of 0.5 ml/min. Unresolved glycan material was eluted after the convex gradient in a single step with 30% acetonitrile in 100 mM sodium phosphate buffer pH 2. Glycan material was detected at 202 nm.

### EXAMPLE 9

### Electronic Microscopy

For scanning electron microscopy (SEM), samples were washed in PBS, prefixed in 2.5% glutaraldehyde in 0.1 M cacodylate buffer for 30 minutes and then rinsed in 0.2 M cacodylate buffer. After post-fixation in 1% osmium tetraoxide (in 0.2 M cacodylate buffer), bacteria were dehydrated in a series of ethanol concentrations. Specimens were critical point dried using carbon dioxide, then coated with gold and examined with a JEOL JSM-6700F SEM.

### EXAMPLE 10

### Minimum Inhibitory Concentration (MIC)

To determine the MIC of different antibiotics, suspensions of H. pylori estimated to contain 10⁸ bacteria/ml (OD600 nm of 0.1) were serially diluted and grown on plates containing various concentrations of amoxicillin. The MIC was defined as the minimal concentration leading to a decrease of 3 log of CFU/ml as compared to growth without antibiotic. Minimum bactericidal concentration (MBC) for amoxicillin was done as follow. Bacteria were grown in increasing concentrations of amoxicillin in liquid culture and OD600 nm was monitored. After 18 hours, CFU/ml counts were determined for each amoxicillin concentration. MBC was defined as the concentration leading to a 3 log decrease of CFU/ml as compared as growth without amoxicillin.

### EXAMPLE 11

### Mice Experiments

Five week old female C57/BL6J mice (Charles River) were intragastrically infected with around 10⁶-5×10⁶ (low dose) and 5×10⁷-10⁸ (high dose) cfu/mouse as previously described (Ferrero et al., 1995; Ferrero et al., 1998). The presence of H. pylori infection in mice was determined by quantitative culture of gastric tissue fragments containing both the antrum and corpus, from mice sacrificed at day 3, 15 and 30 post infection (Ferrero et al., 1998).

### Section 3

To identify useful targets for developing drugs and biologics against H. pylori, the inventors have characterized the roles of the H. pylori lytic transglycosylases. Useful therapeutic agents may be identified by their ability to interfer or block the activities of these important enzymes. Such novel targets are of increasing importance, in view of the growing resistance to antibiotics of H. pylori in the last few decades.

Helicobacter pylori, the etiological agent of gastric diseases such as gastro-duodenal ulcers and adenocarcinoma is becoming also increasingly resistant to the few antibiotics effective in vivo against this infection. Hence, new therapeutic strategies are required to overcome resistance to known antibiotics. The peptidoglycan (PG) is an essential macromolecule surrounding bacteria and responsible for their shape and resistance to turgor pressure. Its central role in cell viability has made the biosynthesis of PG one of the most successful antibiotic targets in bacteria. However, little is known about PG metabolism in H. pylori. A detailed knowledge of the PG metabolism of H. pylori could lead to the development of new antibiotics. From the genome sequences, H. pylori appears to have little redundancy of genes involved in PG metabolism (1-3). H. pylori has all the genetic complement required for the synthesis of PG precursors. Assembly of these precursors in the periplasm requires synthetases and PG hydrolases. H. pylori has three synthetases, penicillin-binding proteins (PBPs) 1, 2 and 3, and three PG hydrolases, two lytic transglycosylases, Slt (HP0645) and MltD (HP1572) and an N-acetylmuramoyl-L-alanyl amidase, AmiA (HP0772).

The aim of the work disclosed in this section was to characterize the two lytic transglycosylases Slt and MltD. The inventors have constructed single and double mutants, and studied their growth and morphological phenotypes. Using reverse phase high-pressure liquid chromatography (HPLC), the inventors analyzed the PG muropeptide composition and glycan strand distribution in the mutants. The results indicate that Slt and MltD are nonredundant lytic transglycosylases with an exo-and endo-type activity, respectively.

### EXAMPLE 12

### Bacteria, Cells and Growth Conditions

Escherichia coli MC1061 (4) and DH5a were used as hosts for the construction and preparation of plasmids. They were cultivated in Luria Bertani solid or liquid media supplemented as appropriate with spectinomycin (100 μg/ml) or kanamycin (40 μg/ml) or both. H. pylori strain 26695 (1) and N6 (5) were used to construct mutants. PG was extracted from strain 26695 and its isogenic mutants. Bacteria were grown microaerobically at 37° C. on blood agar plates or in liquid medium consisting of brain-heart infusion (BHI; Oxoid) with 0.2% β-clycodextrin (Sigma) supplemented with antibiotic-antiflngic mix (6). H. pylori mutants were selected on 20 μg/ml kanamycin or 10 μg/ml gentamycin.

### EXAMPLE 13

### Construction of Mutants

Genes were disrupted as previously described (7). H. pylori mutants were constructed by allelic exchange after transformation with a suicide plasmid carrying the gene of interest interrupted by a non-polar aphA-3 cassette (7) or the miniTn3-Km transposon (8). The double mutant was constructed by disrupting the slt gene with the non-polar gentamycin aacC4 (9) cassette as described below for the non-polar kanamycin cassette. PCR was used to confirm that correct allelic exchange occurred.

Gene replacements were confirmed by sequencing to ensure sequence fidelity. All reagents, enzymes and kits were used according to manufacturers' recommendations. Midiprep (HiSpeed Plasmid Midi kit) and DNA extraction kits (QIAamp DNA Extraction kit) were purchased from QIAGEN. The plasmids, pILL2001 and pILL2002, were used to construct the slt and mltD mutants, respectively. pILL570. Not carrying the genes hp0645 (slt gene) and hp1572 (mltD gene) were used as template for an Expand High Fidelity PCR (Amersham) with oligonucleotides 645-1 (5′-GAUGAUGAUGGTACCGTGTCTGTTGTTTCTAGCATC-3′ (SEQ ID NO: 29) in which the underlined sequence corresponds to the KpnI site) and 645-2 (5′-AUCAUCAUCGGATCCCTAAACGACA TGTTTAACCCCAACATC-3′ (SEQ ID NO: 30) in which the underlined sequence corresponds to the BamHI site) for the slt gene, and, with oligonucleotides 1572-1 (5′-GAUGAUGAUGGTACCTTTTCCTGCTATAAGCCCTTGATG-3′ (SEQ ID NO: 31) in which the underlined sequence corresponds to the KpnI site) and 1572-2 (5′-AUCAUCAUCGGATCCCTTGGAAACCTTAAAATCCTACAACCAC-3′ (SEQ ID NO: 32) in which the underlined sequence corresponds to the BamHI site) for the mltD gene. PCR products were digested with BamHI (Amersham) and KpnI (Amersham), and ligated (T4 DNA ligase, Amersham) with the aphA-3 or the aacC4 non-polar cassette digested with the same endonucleases.

### EXAMPLE 14

### Extraction and Analysis of Lipopolysaccharide

H. pylori lipopolysaccharide (LPS) was extracted from plate cultures by the proteinase K method (10). LPS samples were separated by tricine-sodium dodecyl sulphate (SDS) polyacrylamide gel electrophoresis as described by Lesse and colleagues (11). The LPS was visualized by silver staining (12).

### EXAMPLE 15

### Peptidoglycan Extraction and Analysis

Liquid cultures of H. pylori parental strain and isogenic mutant strains were stopped after various times of growth and chilled in an ice-ethanol bath. The crude murein sacculus was immediately extracted in boiling SDS (4% final). Purification steps and HPLC analyses were done as previously described (13). Mutanolysin (Sigma) digested samples were analyzed by HPLC-on a Hypersil ODS18 reversephase column (250 by 4.6 mm, 3 μm particle size) with a methanol (Fischer, HPLC grade) gradient from 0 to 15% in sodium phosphate buffer pH 4.3 to 5.0. Chromatograms were obtained by monitoring at 206 nm. Each peak was collected, desalted and identified by matrix-assisted laser desorption ionization mass spectrometry (MALDI-MS) as described previously (14). Glycan chain analysis was done as previously described (15,16).

Briefly, H. pylori PG was digested with purified human serum amidase kindly provided by Waldemar Vollmer. The digestion was done in 50 mM Tris-HCl pH 7.9, 5 mM MgCl₂, 0.02% NaN3. Soluble material was first purified on a MonoS (HR5/5) column (Amersham Pharmacia) using a 10 mM sodium phosphate buffer pH 2. Glycans eluted with the flow-through and were collected. Free peptides were eluted by one step using 1 M NaCl, 10 mM sodium phosphate buffer pH 2. The runs performed at room temperature using a flow of 1 ml/nm. Amino acid and amino sugar analysis was performed on the purified glycan fraction and the free peptide fraction to ensure purity of each fraction confirming that complete digestion had occurred. Purified glycans were analyzed by reverse phase HPLC using a 5 μm Nucleosil 300 C18 column (250×4.6 mm) at 50° C. A convex gradient from 0 to 10.5% acetonitrile (˜4 curve of the Shimadzu CLASS-VP software) in 100 mM sodium phosphate buffer pH 2 was used over 90 minutes at a flow rate of 0.5 ml/min. Unresolved glycan material was eluted after the convex gradient in a single step with 30% acetonitrile in 100 mM sodium phosphate buffer pH 2. Glycan material was detected at 202 nm. Slt70 digestion of H. pylori PG. PG from strain 26695 slt/mltD was incubated in 300 mM sodium acetate buffer pH 4.5 with His6-tagged Slt70 (1 ng/ml) for different time periods (1, 5 minutes and 48 hours) at 37° C. The reaction was stopped by boiling the sample for 5 minutes. Muropeptides were separated as indicated above and identified by MALDI-MS as described previously (14).

### EXAMPLE 16

### Electron Microscopy

For transmission electron microscopy, samples were washed in PBS, prefixed in 2.5% glutaraldehyde in PBS buffer for 30 minutes. After postfixation in 2% molybdate (in PBS buffer), bacteria were examined with a JEOL Jem 1010.

Characterization of slt and mltD Mutants

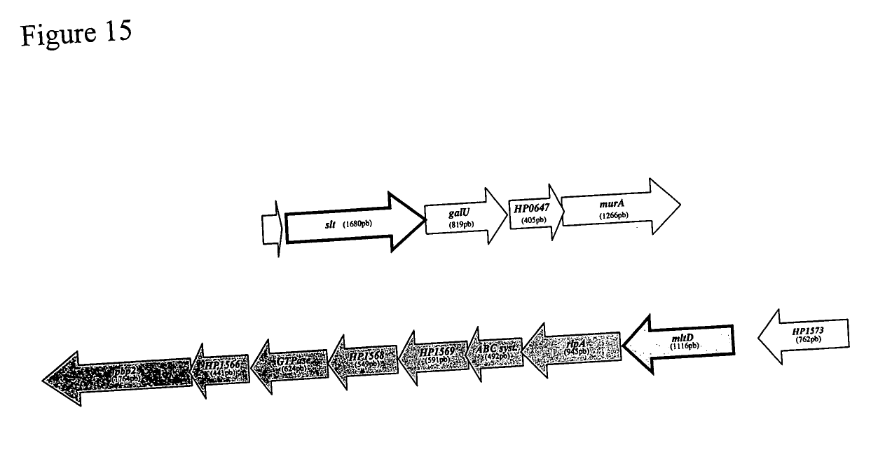

The inventors constructed slt and mltD mutants in strain 26995 and N6 background using both the miniTn3-Km transposon (17) and a non-polar kanamycin cassette (7). The slt and mltD genes are organized in putative operons (FIG. 15). Therefore, to study their role in H. pylori PG metabolism the inventors had to insure that their inactivation would not create polar effects on downstream genes such as galU in the case of slt. GalU catalyzes the conversion of glucose-1-phosphate into UDP-glucose. UDP-glucose is a substrate of GalE, which generates UDP-galactose, an amino sugar precursor in the synthesis of LPS. Interference with GalU activity thus leads to a rough LPS.

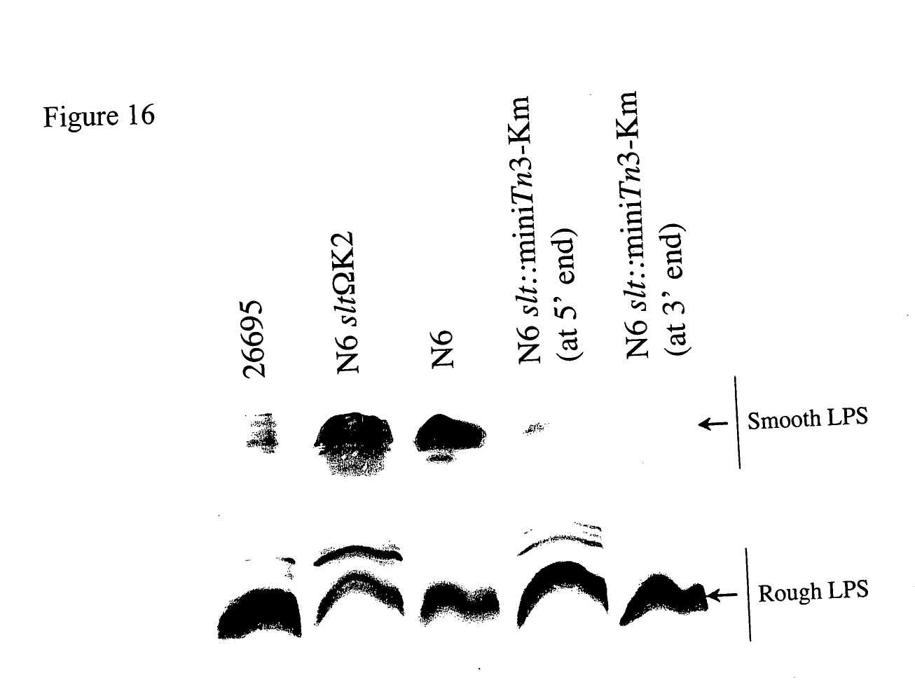

The inventors analyzed the LPS phenotype of strain 26995 (FIG. 16) and found it already had a rough LPS. Therefore, to observe eventual polar effects either of the miniTn3 transposon or the non-polar kanamycin cassette, the inventors constructed slt mutants in strain N6, which presents smooth LPS (FIG. 16). As shown in FIG. 16, while the two individual miniTn3 mutants with insertions either at the 5′ or the 3′ end of slt gene affected the smooth LPS phenotype, the non-polar kanamycin cassette had no effect on the LPS phenotype showing the non-polar nature of this mutant. The same approach was used for analyzing the mltD mutants. The mltD gene appears to be the first gene of an eight genes operon (FIG. 15). It includes homologues of the rare lipoprotein A (HP1571), the inner membrane component of an ABC transporter system (HP1570), a GTPase involved in cell division (HP1567) and penicillin-binding protein 2 (HP1565). Based on these results, an expected polar effect on down stream genes would be a cell division phenotype.

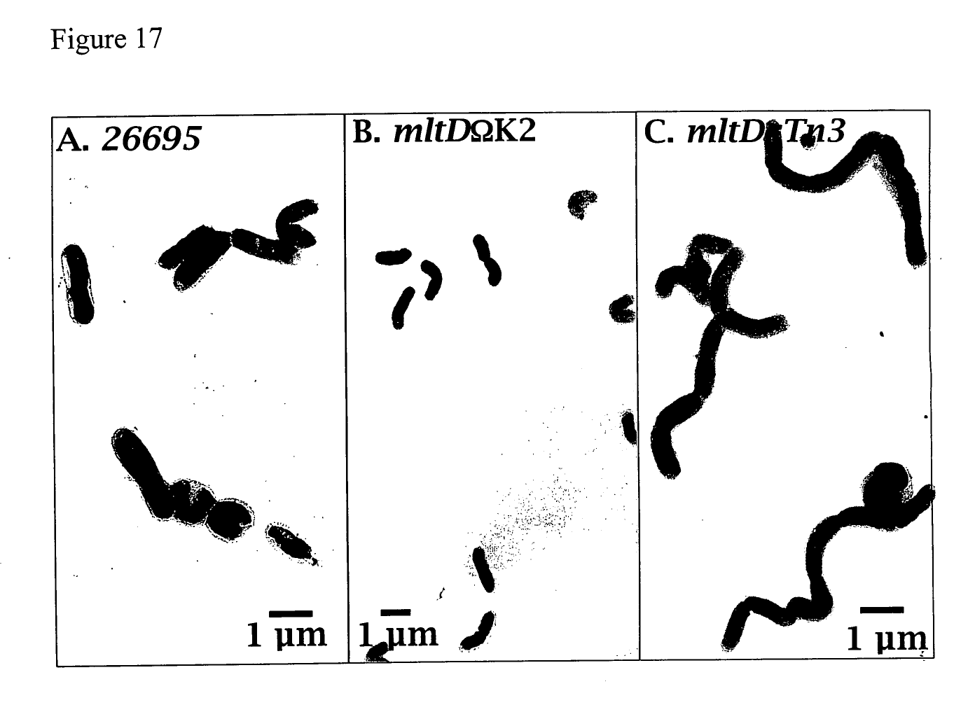

Morphological analysis of the miniTn3 and kanamycin mutants is illustrated in FIG. 17. While miniTn3 insertions at the 3′ end of mltD led to a chaining phenotype, mltD inactivation with the kanamycin cassette showed normal bacillary morphology. Hence the inventors confirmed the non-polar nature of both slt and mltD mutants.

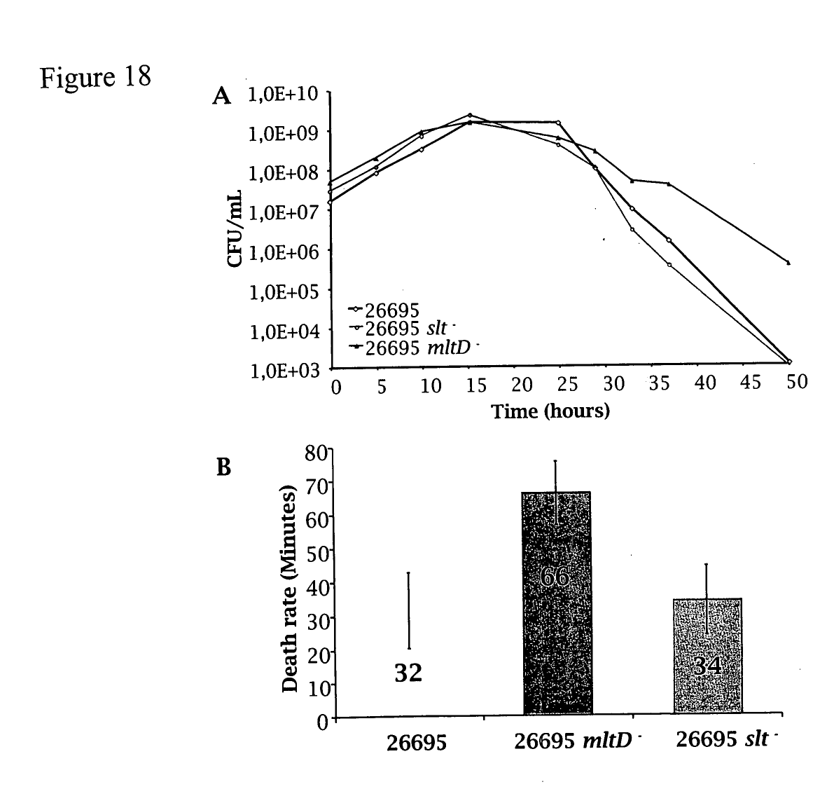

Next, the inventors studied any growth defects the non-polar mutants might have. Hence the inventors followed the number of colonies forming units during exponential growth and stationary phase for the wild type strain 26695 and its two isogenic slt and mltD mutants. As shown in FIG. 18, the three strains had the same growth rate. However, after entry into stationary phase, the mltD mutant maintained longer its viability. This result was reflected by a lower rate of death (FIG. 18B) of the mltD mutant. Muropeptide composition. Since Slt and MltD are predicted to be involved in PG metabolism, in particular, in PG degradation, the inventors purified the PG of the parental strain and of the two mutants to analyze their muropeptide composition by reverse phase HPLC. The results are presented in supplementary tables 1, 2 and 3, which correspond to the muropeptide composition of the three strains at different time points of their growth (8 h, 24 h and 48 h, respectively). A difference that was growth dependent but strain independent concerned the increase of the GMdipeptide motif when H. pylori entered in stationary phase. This modification is discussed in another manuscript (Chaput et al.).

Globally, the mltD mutant presented a similar muropeptide composition as the parental strain 26695. Some differences were apparent such as a modest decrease of anhydromuropeptides

TABLE 1

| | | | Anhydro- | Average glycan |
|---|---|---|---|---|
| Strain | Monomers | Dimers | muropeptides | chain length |
| 8 hours | | | | |
| 26695 | 70.7% ± 1.8 | 29.3% ± 1.8 | 14.2% ± 1.1 | 9.2 ± 0.7 |
| mltD⁻ | 73.1% ± 1.6 | 26.9% ± 1.6 | 12.0% ± 0.9 | 10.2 ± 0.3 |
| slt⁻ | 76.3% ± 2.0 | 23.7% ± 2.0 | 9.9% ± 1.4 | 13.1 ± 0.1 |
| slt⁻/mltD⁻ | 78.8% | 21.2% | 5.7% | 15.7 |
| 24 hours | | | | |
| 26695 | 68.0% ± 0.8 | 32.0% ± 0.8 | 14.9% ± 0.8 | 9.0 ± 0.4 |
| mltD⁻ | 71.2% ± 0.4 | 28.8% ± 0.4 | 14.5% ± 0.7 | 8.9 ± 0.4 |
| slt⁻ | 73.3% ± 0.4 | 26.7% ± 0.4 | 10.6% ± 1.5 | 11.6 ± 1.4 |
| slt⁻/mltD⁻ | 77.9% | 22.1% | 5.4% | 16.6 |
| 48 hours | | | | |
| 26695 | 68.9% ± 2.2 | 31.1% ± 2.2 | 13.3% ± 4.9 | 7.9 |
| mltD⁻ | ND | ND | ND | ND |
| slt⁻ | 74.5% | 25.5% | 9.8% | 12.8 |
| slt⁻/mltD⁻ | 79.7% | 20.3% | 4.2% | 20.0 |

TABLE 2

| Strain | Dipeptides | Tripeptides | Tetrapeptides | TetraGlypeptides | Pentapeptides |
|---|---|---|---|---|---|
| 8 hours | | | | | |
| 26695 | 3.2% ± 1.0 | 19.1% ± 0.8 | 45.4% ± 1.7 | 6.6 ± 1.9 | 54.9% ± 1.7 |
| mltD⁻ | 3.7% ± 1.5 | 23.0% ± 0.3 | 42.6% ± 2.1 | 5.5 ± 0.8 | 52.2% ± 2.3 |
| slt⁻ | 4.7% ± 1.0 | 28.3% ± 0.9 | 32.0% ± 1.8 | 5.2 ± 0.7 | 53.4% ± 1.5 |
| slt⁻/mltD⁻ | 6.4% | 36.6% | 30.1% | 6.2 | 41.8% |
| 24 hours | | | | | |
| 26695 | 9.5% ± 0.6 | 18.9% ± 0.1 | 47.8% ± 1.2 | 5.9 ± 0.3 | 49.8% ± 0.6 |
| mltD⁻ | 15.4% ± 0.6 | 18.3% ± 0.5 | 44.8% ± 0.5 | 5.4 ± 0.5 | 45.0% ± 0.3 |
| slt⁻ | 8.2% ± 1.1 | 28.5% ± 0.7 | 34.7% ± 0.2 | 5.3 ± 0.5 | 50.1% ± 0.0 |
| slt⁻/mltD⁻ | 10.5% | 36.2% | 34.5% | 7.6 | 33.2% |
| 48 hours | | | | | |
| 26695 | 16.6% ± 7.6 | 9.9% ± 0.6 | 45.5% ± 4.2 | 6.5 ± 0.1 | 52.6% ± 0.5 |
| mltD⁻ | ND | ND | ND | ND | ND |
| slt⁻ | 18.3% | 17.0% | 35.7% | 6.1 | 48.5% |
| slt⁻/mltD⁻ | 18.7% | 24.8% | 39.2% | 8.6 | 28.9% |

### Tables 1-3 and 2

Another difference concerned the proportion of monomeric versus dimeric muropeptides. Hence, the mltD mutant had a modest increase of the monomeric muropeptides indicating that the degree of cross-linking of the mltD. The slt mutant showed the same global trend in terms of changes of muropeptide composition as the mltD mutant but to a much greater extent. The decrease in anhydromuropeptides and degree of cross-linking was more pronounced in the sit mutant. Furthermore, the slt mutant showed a marked accumulation of muropeptides carrying a tripeptide as a stem peptide. This increase was inversely proportional to the decrease of muropeptides carrying tetrapeptides and tetra-glycine peptides (Table 2). The differences in the degree of cross-linking, in anhydromuropeptides and in GMtripeptide were further exacerbated in the slt/mltD double mutant.

Most importantly, despite the fact that slt and mltD are the only two homologues of known lytic transglycosylases in H. pylori genome, the double mutant still presented in its muropeptide composition anhydromuropeptide structures. These were mainly composed of the GanhMpenta and GanhM-tri-tetra-GM, GanhM-penta-tetra-GM muropeptides (supplementary Tables 1, 2 and 3).

SUPPLEMENTARY TABLE 1
Molar percentage of each muropeptide for the parental strain
26695 and its single and double mutants at 8 hours of growth
(values calculated according to Glauner (13).

| | | | | | |
|---|---|---|---|---|---|
| Peaks | Muropeptides | 26695 | mltD | slt | slt/mltD |

Monomers

| | | | | | |
|---|---|---|---|---|---|
| 1 | GM-tri | 12.8%_{±0.6} | 16.1%_{±0.6} | 20.6%_{±1.0} | 29.2% |
| 2 | GM-tetra | 8.8%_{±1.9} | 9.5%_{±0.9} | 4.3%_{±1.6} | 4.9% |
| 3 | GM-tetraGly | 5.1%_{±2.2} | 3.8%_{±0.8} | 3.8%_{±0.7} | 4.5% |
| 4 | GM-di | 3.2%_{±1.0} | 3.7%_{±1.5} | 4.7%_{±1.0} | 6.4% |
| 5 | GM-penta | 37.7%_{±1.9} | 37.5%_{±1.6} | 39.8%_{±1.4} | 31.8% |

Dimers

| | | | | | |
|---|---|---|---|---|---|
| 6 | GM-tri-tetra-MG | 3.4%_{±0.3} | 4.0%_{±0.3} | 4.7%_{±0.3} | 5.1% |
| 7 | GM-tetra-Gly-tetra-MG | 1.5%_{±0.5} | 1.6%_{±0.9} | 1.4%_{±0.3} | 1.7% |
| 8 | GM-tetra-tetra-MG | 4.1%_{±0.3} | 3.7%_{±0.7} | 2.8%_{±0.6} | 2.9% |
| 9 | GM-penta-tetra-MG | 9.1%_{±0.6} | 8.1%_{±0.6} | 8.0%_{±0.9} | 5.9% |

Anhydromuropeptides

| | | | | | |
|---|---|---|---|---|---|
| 10 | GanhM-penta | 3.1%_{±0.6} | 2.5%_{±0.7} | 3.1%_{±1.1} | 2.1% |
| 11 | GanhM-tri-tetra-MG | 1.4%_{±0.4} | 1.4%_{±0.5} | 2.0%_{±0.2} | 1.6% |
| 12 | GM-tri-tetra-anhMG | 1.5%_{±0.4} | 1.5%_{±0.3} | 1.0%_{±0.2} | 0.8% |
| 13 | GanhM-tetra-tetra-MG | 2.0%_{±0.3} | 1.4%_{±0.3} | 0.9%_{±0.2} | 0.8% |
| 14 | GM-tetra-tetra-anhMG | 1.2%_{±0.4} | 1.0%_{±0.3} | 0.4%_{±0.1} | 0.4% |
| 15 | GanhM-penta-tetra-MG | 5.0%_{±0.5} | 4.1%_{±0.6} | 2.5%_{±0.5} | 2.1% |

SUPPLEMENTARY TABLE 2
Molar percentage of each muropeptide for the parental strain
26695 and its single and double mutants at 24 hours of growth
(values calculated according to Glauner, (13).

| | | | | | |
|---|---|---|---|---|---|
| Peaks | Muropeptides | 26695 | mltD | slt | slt/mltD |

Monomers

| | | | | | |
|---|---|---|---|---|---|
| 1 | GM-tri | 10.6%_{±0.1} | 10.0%_{±0.1} | 19.8%_{±0.9} | 28.1% |
| 2 | GM-tetra | 9.0%_{±0.3} | 9.0%_{±0.3} | 3.6%_{±0.6} | 7.6% |
| 3 | GM-tetraGly | 3.6%_{±0.6} | 3.2%_{±0.0} | 2.8%_{±0.7} | 5.8% |
| 4 | GM-di | 9.5%_{±0.6} | 15.4%_{±0.6} | 8.2%_{±1.1} | 10.5% |
| 5 | GM-penta | 33.8%_{±0.1} | 32.5%_{±0.1} | 36.0%_{±1.0} | 24.0% |

Dimers

| | | | | | |
|---|---|---|---|---|---|
| 6 | GM-tri-tetra-MG | 4.1%_{±0.6} | 3.6%_{±0.1} | 5.3%_{±0.2} | 5.3% |
| 7 | GM-tetra-Gly-tetra-MG | 2.3%_{±0.3} | 2.2%_{±0.3} | 2.5%_{±0.6} | 1.9% |
| 8 | GM-tetra-tetra-MG | 3.8%_{±0.1} | 3.6%_{±0.2} | 3.1%_{±0.2} | 3.6% |
| 9 | GM-penta-tetra-MG | 8.4%_{±0.1} | 6.1%_{±0.6} | 8.1%_{±0.2} | 5.9% |

Anhydromuropeptides

| | | | | | |
|---|---|---|---|---|---|
| 10 | GanhM-penta | 1.5%_{±0.0} | 1.1%_{±0.3} | 3.1%_{±1.1} | 1.9% |
| 11 | GanhM-tri-tetra-MG | 2.4%_{±0.3} | 2.7%_{±0.2} | 2.4%_{±0.0} | 1.9% |
| 12 | GM-tri-tetra-anhMG | 1.7%_{±0.1} | 2.0%_{±0.2} | 1.0%_{±0.0} | 0.9% |
| 13 | GanhM-tetra-tetra-MG | 1.8%_{±0.0} | 1.7%_{±0.1} | 0.8%_{±0.1} | 0.8% |
| 14 | GM-tetra-tetra-anhMG | 1.3%_{±0.1} | 1.7%_{±0.1} | 0.5%_{±0.1} | 0.4% |
| 15 | GanhM-penta-tetra-MG | 6.2%_{±0.4} | 5.3%_{±0.2} | 3.2%_{±0.3} | 1.4% |

SUPPLEMENTARY TABLE 3
Molar percentage of each muropeptide for the parental strain
26695 and its single and double mutants at 48 hours of growth
(values calculated according to Glauner, (13).

| | | | | | |
|---|---|---|---|---|---|
| Peaks | Muropeptides | 26695 | mltD | slt | slt/mltD |

Monomers

| | | | | | |
|---|---|---|---|---|---|
| 1 | GM-tri | 2.7% | N.D. | 8.7% | 16.7% |
| 2 | GM-tetra | 1.2% | N.D. | 5.7% | 14.2% |
| 3 | GM-tetraGly | 3.9% | N.D. | 3.6% | 6.8% |
| 4 | GM-di | 21.9% | N.D. | 18.3% | 18.7% |
| 5 | GM-penta | 36.3% | N.D. | 36.4% | 21.5% |

Dimers

| | | | | | |
|---|---|---|---|---|---|
| 6 | GM-tri-tetra-MG | 2.7% | N.D. | 5.0% | 5.5% |
| 7 | GM-tetraGly-tetra-MG | 2.5% | N.D. | 2.4% | 1.8% |
| 8 | GM-tetra-tetra-MG | 4.4% | N.D. | 3.1% | 3.5% |
| 9 | GM-penta-tetra-MG | 7.5% | N.D. | 6.9% | 5.3% |

Anhydromuropeptides

| 10 | GanhM-penta | 1.2% | N.D. | 1.7% | 1.8% |
|---|---|---|---|---|---|
| 11 | GanhM-tri-tetra-MG | 2.6% | N.D. | 2.3% | 1.7% |
| 12 | GM-tri-tetra-anhMG | 1.5% | N.D. | 0.9% | 0.9% |
| 13 | GanhM-tetra-tetra-MG | 2.3% | N.D. | 1.0% | 0.8% |
| 14 | GM-tetra-tetra-anhMG | 1.9% | N.D. | 0.4% | 0.5% |
| 15 | GanhM-penta-tetra-MG | 7.3% | N.D. | 3.5% | 0.3% |
| Supplementary FIG. 1. Analysis of the glycan strand length distribution of the parental strain 26695 and its slt and mltD single mutants. The peak number corresponds to the number of disaccharide repeating units of each glycan strand species. Glycans with more than 26 disaccharide repeating units are eluted as a single peak at the end of the chromatogram by a single 30% acetonitrile step. Note that the | | | | | |
| # scale of the left and right Y axis is different to accommodate the single peak at the end of the chromatogram. The relative intensity of each peak as presented in FIG. 5 corresponds to the ration of each peak area over the total UV glycan strand peak area. The relative percentage of the single peak of the glycan strands >26 disaccharide repeating units is presented to the right of the corresponding peak. | | | | | |

The other anhydromuropeptides were found only in trace amounts. Since anhydromuropeptides represent the ends of the glycan chains, the inventors estimated the overall average length of the glycan chains of the parental strain 26695, the two single mutants and the double mutant. While the parental strain and the mltD mutant presented an average glycan chain length of around 8-10 disaccharide repeating units with a moderate decrease in stationary phase, the slt mutant had a marked increase in the average length, which varied between 11.6 and 13.1 disaccharide repeating units. The increase in average length was clearly increased in the double mutant (between 15.7 and 20 disaccharide repeating units).

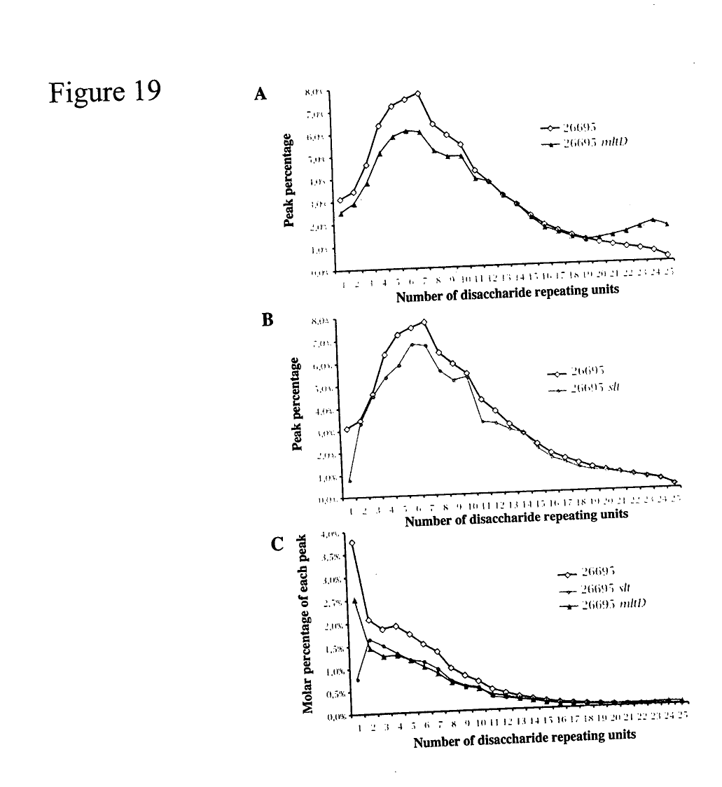

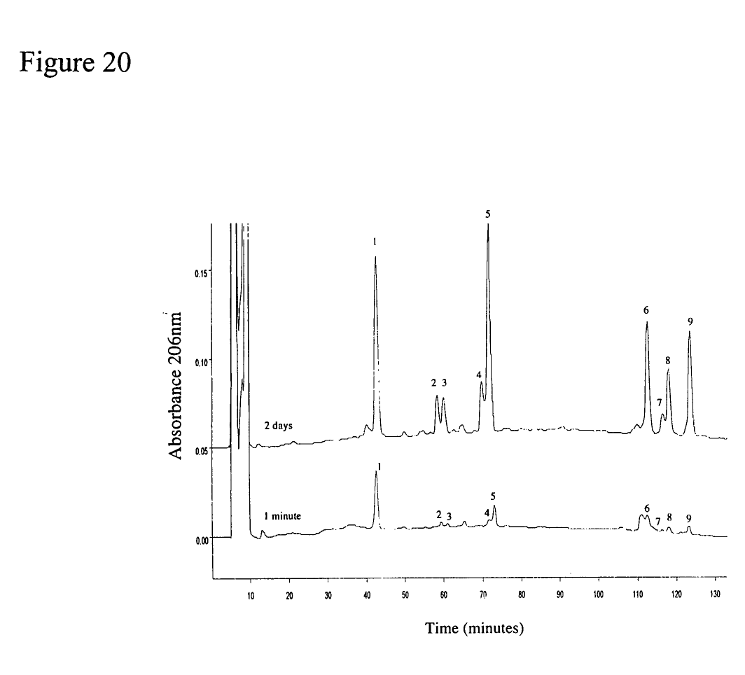

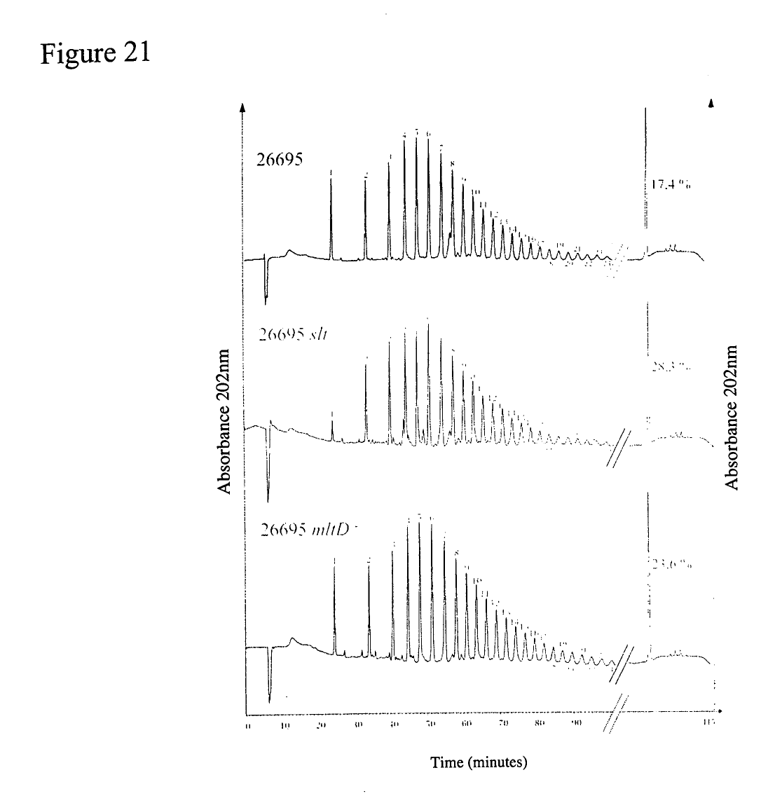

Glycan chain length analysis. Next, the inventors were interested in characterizing by a more detailed methodology the glycan strands of the parental strain and the single mutants. The inventors digested the purified peptidoglycan of each strain with the human serum amidase, and separated the glycan strands from the free peptides by a first chromatography using a MonoS column. The glycan fraction eluted exclusively with the flowthrough while the free peptides were retained on the column. The purified glycan fraction was analyzed by reverse phase HPLC (FIG. 21). The profile is reminiscent of the glycan strand analysis of E. coli (15). Analysis of the glycan strand distribution was restricted to 8 h of growth since the human serum amidase was not able to cleave the GM-dipeptide accumulated in stationary phase (24 h and 48 h) as previously described (18). The proportion of each peak was calculated based on the total UV absorbing material (FIG. 19 and FIG. 21). Several differences were observed between the parental strain and the two single mutants. Both mutants showed a marked increase of very long glycan chains (=26 disaccharide repeating units. The proportion of these glycan species increased from 17.4% in the parental strain to 23.6% and 28.3% in the mltD and slt mutants, respectively. Analysis of the UV proportion of each glycan species and their corresponding molar percentage, the inventors observed that the mltD mutant presented a marked decrease of the short glycan species (1 to 11 disaccharide repeating units, FIGS. 19A and C) and an inversely increase of glycan species with more than >19 disaccharide repeating units (FIG. 19A). These results suggest that mltD might have an endo transglycosylase activity. The slt mutant also showed a marked decrease of the very short glycan strands (FIGS. 19B and C). However, the major decreased was due to an almost complete absence of the disaccharide species (peak 1) from the slt mutant, which was still present in the mltD mutant. This suggests that in contrast to MltD, Slt would appear to cut preferentially at the ends of the glycan strands to generate free disaccharide units, suggesting that Slt would carry an exo-type activity. Slt70 digestion and GM-tripeptide localization. From the muropeptide and glycan strand analysis, the inventors observed that the slt mutant accumulated the GMtripeptide motif and generated significantly less of the disaccharide GanhM glycan species. Since the inventors do not observe in the PG the monomeric GanhM-tripeptide under any condition tested, this suggests that the GM-tripeptides are either at the nonreducing end of the glycan strands or in the middle of the glycan strands. However, since the slt mutant generates less GanhM, this suggests that Slt functions as an exoenzyme. Hence, the accumulation of the GM-tripeptides is likely due to a preferential location at the non-reducing ends of the glycan strands. To test this hypothesis, the inventors used the E. coli Stl70 lytic transglycosylase which has been shown to be an exo-enzyme (19). The inventors digested the same amount of H. pylori PG with Slt70 during very brief period (1 and 5 minutes) and after two days, and analyzed the nature of the Slt70 generated muropeptides by HPLC. As shown by FIG. 20, Slt70 generated preferentially the GanhMtripeptide after 1 minute incubation, clearly indicating that the GM-tripeptides are located preferentially at the nonreducing end of the glycan strands. Similar results were obtained after 5 minutes of digestion.

From the genome analysis, two genes, slt and mltD, are the only ones that encode proteins presenting a lytic transglycosylase domain (2). The slt gene is predicted to encode a 560 amino acid long protein with a classical signal peptide. Slt presents a SLT domain at the C-terminal end of the protein with a 34% identity to E. coli Slt70 . The rest of the protein has no homology in the databases. In contrast, the mltD gene encodes a shorter protein (372 aa) with a classical signal peptide, SLT domain at its N-terminal end and a single LysM domain at the C-terminal end. The STL domain shows 31% identity to Slt70 . Hence, both proteins were predicted to function as lytic transglycosylases. Analyses of the muropeptide composition and of the glycan strand distribution of the single and double mutants suggest that both proteins are lytic transglycosylases with non-redundant functions.

Inactivation of each gene resulted in a substantial decrease of the anhydromuropeptides. Since these muropeptides species represent the products of lytic transglycosylase activities, the results suggested that both proteins function as such. The muropeptide composition analysis of the single mutants and the double mutant (supplementary Tables 1 to 3 and Table 1) shows that the total percentage of anhydromuropeptides results from the additive effect of Slt and MltD. Hence, the decrease in anhydromuropeptides in the double mutant compared to the parental strain corresponds to the differential of anhydromuropeptides in the slt mutant plus the one in the mltD mutant. This suggests that Slt and MltD generate anhydromuropeptides independently j of the each other lytic transglycosylase, and, that each protein has a different physiological role.

Glycan strand analysis of the slt and mltD mutants confirms this hypothesis. While both mutants accumulate very long glycan strands, each mutant seems to act by a different mechanism. While the slt mutant increases the length of its glycan strands by generating less of the very short glycan strands, in particular, the disaccharide GanhM, the mltD mutant does it by reducing the amount of glycan strands with sizes reaching up to 10-11 disaccharide units and a gradual increase of glycan strands with a more than 19 disaccharide repeating units (see FIG. 19). The distinct pattern in glycan strand distribution of the slt and mltD mutants suggests that Slt and MltD would function as an exo-type and an endotype lytic transglycosylase, respectively. The inferred type of activity of Slt and MltD fits with the increased fitness of the mltD mutant seen during stationary phase growth. As an endo-type lytic transglycosylase, MltD would have a grater impact on the PG layer stability that Slt.

In contrast, Slt would function primarily in releasing anhydromuropeptides during PG turnover. The slt mutant has a marked effect on the proportion of GM-tripeptide, which is massively accumulated in this mutant PG layer. The inventors can interpret this result either as a consequence of Slt substrate specificity and/or as a result of a particular localization of tripeptides along the glycan strands. Both hypotheses are possible and might occur simultaneously. From the glycan strand analysis Slt appears to be an exo-type enzyme. If the accumulation of GM-tripeptide resulted exclusively from substrate specificity, the inventors would expect to observe in wild type strains the presence of GanhM-tripeptides. However, these structures are completely absent from H. pylori PG. Therefore, the inventors infer that these are always generated as turnover products and immediately released from the PG layer. The only way to generate readily soluble GanhM-tripeptides is whether the GM-tripeptide structures are exclusivelyat the non-reducing ends of glycan strands. In the absence of Slt, these accumulate in the PG layer of the mutant exclusively as GMtripeptides.

The inventors confirmed the particular localization of the GM-tripeptides at the ends of glycan strands by partially digesting H. pylori PG with the exo-type lytic transglycosylase Slt70 from E. coli. Slt70 preferentially released GanhMtripeptide after very short incubations (1 minute; see FIG. 20). Interestingly, in the double mutant, the inventors still observed the presence of anhydromuropeptides. The inventors can explain this result by either 1) the presence of a novel class of lytic transglycosylases to be identified or 2) the glycosyltransferase domain of the bifunctional class A highmolecular weight (HMW) PBP1 is capable of generating the intra-molecular anhydrous bond as a nascent glycan strand is released from the undecaprenylphosphate anchor. Further work is required to distinguish between the two hypotheses. Nevertheless, the double mutant presents almost exclusively the GanhM-pentapeptide and the GanhM-penta-tetra-GM dimeric muropeptide. This indicates that the reducible ends of glycan strands are enriched in intact stem pentapeptides consistent with de novo synthesis favoring a role for PBP1 in the generation of the anhydromuropeptides.

Finally, the inventors' results indicate the in H. pylori, synthesis of new glycan strands is initiated by a GM-tripeptide and terminates by a GM-pentapeptide. The GM-tripeptide might originate from a classical lipid II precursor immediately processed from a pentapeptide to a tripeptide. Processing might occur either by an L,D-endopeptidase or by consecutive digestion by a D,D-and L,D-carboxypeptidase. H. pylori lacks classical D,D-carboxypeptidases although the inventors cannot exclude that the three HMW PBPs would function as such. However, no homologue of known L,D-peptidase is found in the H. pylori genome, which would require a novel class of L,D-peptidases.

Alternatively, the GM-tripeptide might originate directly from the PG precursor pool as lipid precursor and be used to initiate glycan strand elongation. In fact, a precursor pool origin for the GM-tripeptide could be an elegant mechanism to naturally regulate the glycan strand length. The glycan strand length distribution would be regulated by the precursor pool of UDP-M-tripeptide rather by the synthetases or the PG hydrolases.

### REFERENCES FOR SECTION 1

1. Tominaga K, Hamasaki N, Watanabe T, Uchida T, Fujiwara Y, et al. (1999) Effect of culture conditions on morphological changes of Helicobacter pylori. J Gastroenterol 34 Suppl 11: 28-31.
2. Donelli G, Matarrese P, Fiorentini C, Dainelli B, Taraborelli T, et al. (1998) The effect of oxygen on the growth and cell morphology of Helicobacter pylori. FEMS Microbiol Lett 168: 9-15.
3. Berry V, Jennings K, Woodnutt G (1995) Bactericidal and morphological effects of amoxicillin on Helicobacter pylori. Antimicrob Agents Chemother 39: 1859-1861.
4. DeLoney C R, Schiller N L (1999) Competition of various beta-lactam antibiotics for the major penicillin-binding proteins of Helicobacter pylori: antibacterial activity and effects on bacterial morphology. Antimicrob Agents Chemother 43: 2702-2709.
5. Noach L A, Rolf T M, Tytgat G N (1994) Electron microscopic study of association between Helicobacter pylori and gastric and duodenal mucosa. J Clin Pathol 47: 699-704.
6. Chan W Y, Hui P K, Leung K M, Chow J, Kwok F, et al. (1994) Coccoid forms of Helicobacter pylori in the human stomach. Am J Clin Pathol 102: 503-507.
7. Nilsson H O, Blom J, Abu-Al-Soud W, Ljungh A A, Andersen L P, et al. (2002) Effect of cold starvation, acid stress, and nutrients on metabolic activity of Helicobacter pylori. Appl Environ Microbiol 68: 11-19.
8. Cellini L, Robuffo I, Di Campli E, Di Bartolomeo S, Taraborelli T, et al. (1998) Recovery of Helicobacter pylori ATCC43504 from a viable but not culturable state: regrowth or resuscitation? Apmis 106: 571-579.
9. Cole S P, Cirillo D, Kagnoff M F, Guiney D G, Eckmann L (1997) Coccoid and spiral Helicobacter pylori differ in their abilities to adhere to gastric epithelial cells and induce interleukin-8 secretion. Infect Immun 65: 843-846.
10. Gribbon L T, Barer M R (1995) Oxidative metabolism in nonculturable Helicobacter pylori and Vibrio vulnificus cells studied by substrate-enhanced tetrazolium reduction and digital image processing. Appl Environ Microbiol 61: 3379-3384.
11. Adams B L, Bates T C, Oliver J D (2003) Survival of Helicobacter pylori in a natural freshwater environment. Appl Environ Microbiol 69: 7462-7466.
12. Cole S P, Kharitonov V F, Guiney D G (1999) Effect of nitric oxide on Helicobacter pylori morphology. J Infect Dis 180: 1713-1717.
13. Shirai M, Kakada J, Shibata K, Morshed M G, Matsushita T, et al. (2000) Accumulation of polyphosphate granules in Helicobacter pylori cells under anaerobic conditions. J Med Microbiol 49: 513-519.
14. Taneera J, Moran A P, Hynes S O, Nilsson H O, Al-Soud W, et al. (2002) Influence of activated charcoal, porcine gastric mucin and beta-cyclodextrin on the morphology and growth of intestinal and gastric Helicobacter spp. Microbiology 148: 677-684.
15. Mizoguchi H, Fujioka T, Kishi K, Nishizono A, Kodama R, et al. (1998) Diversity in protein synthesis and viability of Helicobacter pylori coccoid forms in response to various stimuli. Infect Immun 66: 5555-5560.
16. Bumann D, Habibi H, Kan B, Schmid M, Goosmann C, et al. (2004) Lack of stage-specific proteins in coccoid Helicobacter pylori cells. Infect Immun 72: 6738-6742.
17. Nilsson I, Utt M, Nilsson H O, Ljungh A, Wadstrom T (2000) Two-dimensional electrophoretic and immunoblot analysis of cell surface proteins of spiral-shaped and coccoid forms of Helicobacter pylori. Electrophoresis 21: 2670-2677.
18. Figueroa G, Faundez G, Troncoso M, Navarrete P, Toledo M S (2002) Immunoglobulin G antibody response to infection with coccoid forms of Helicobacter pylori. Clin Diagn Lab Immunol 9: 1067-1071.
19. Monstein H J, Jonasson J (2001) Differential virulence-gene mRNA expression in coccoid forms of Helicobacter pylori. Biochem Biophys Res Commun 285: 530-536.
20. Takeuchi H, Shirai M, Akada J K, Tsuda M, Nakazawa T (1998) Nucleotide sequence and characterization of cdrA, a cell division-related gene of Helicobacter pylori. J Bacteriol 180: 5263-5268.
21. Shimomura H, Hayashi S, Yokota K, Oguma K, Hirai Y (2004) Alteration in the composition of cholesteryl glucosides and other lipids in Helicobacter pylori undergoing morphological change from spiral to coccoid form. FEMS Microbiol Lett 237: 407-413.
22. Cabeen M T, Jacobs-Wagner C (2005) Bacterial cell shape. Nat Rev Microbiol 3: 601-610.
23. Costa K, Bacher G, Allmaier G, Dominguez-Bello M G, Engstrand L, et al. (1999) The morphological transition of Helicobacter pylori cells from spiral to coccoid is preceded by a substantial modification of the cell wall. J Bacteriol 181: 3710-3715.
24. Goodwin A, Kersulyte D, Sisson G, Veldhuyzen van Zanten S J, Berg D E, et al. (1998) Metronidazole resistance in Helicobacter pylori is due to null mutations in a gene (rdxA) that encodes an oxygen-insensitive NADPH nitroreductase. Mol Microbiol 28: 383-393.
25. Girardin S E, Boneca I G, Cameiro L A, Antignac A, Jehanno M, et al. (2003) Nod1 detects a unique muropeptide from gram-negative bacterial peptidoglycan. Science 300: 15841587.
26. Viala J, Chaput C, Boneca I G, Cardona A, Girardin S E, et al. (2004) Nod1 responds to peptidoglycan delivered by the Helicobacter pylori cag pathogenicity island. Nat Immunol 5: 1166-1174.
27. Boneca I G, de Reuse H, Epinat J C, Pupin M, Labigne A, et al. (2003) A revised annotation and comparative analysis of Helicobacter pylori genomes. Nucleic Acids Res 31: 17041714.
28. Heidrich C, Templin M F, Ursinus A, Merdanovic M, Berger J, et al. (2001) Involvement of N-acetylmuramyl-L-alanine amidases in cell separation and antibiotic-induced autolysis of Escherichia coli. Mol Microbiol 41: 167-178.
29. Ishikawa S, Kawahara S, Sekiguchi J (1999) Cloning and expression of two autolysin genes, cwIU and cwIV, which are tandemly arranged on the chromosome of Bacillus polymyxa var. colistinus. Mol Gen Genet 262: 738-748.
30. Wang Z M, Li X, Cocklin R R, Wang M, Wang M, et al. (2003) Human peptidoglycan recognition protein-L is an N-acetylmuramoyl-L-alanine amidase. J Biol Chem 278: 49044-49052.
31. Spratt B G, Boyd A, Stoker N (1980) Defective and plaque-forming lambda transducing bacteriophage carrying penicillin-binding protein-cell shape genes: genetic and physical mapping and identification of gene products from the lip-dacA-rodA-pbpA-leuS region of the Escherichia coli chromosome. J Bacteriol 143: 569-581.
32. Pinho M G, Errington J (2005) Recruitment of penicillin-binding protein PBP2 to the division site of Staphylococcus aureus is dependent on its transpeptidation substrates. Mol Microbiol 55: 799-807.
33. Segal E D, Falkow S, Tompkins L S (1996) Helicobacter pylori attachment to gastric cells induces cytoskeletal rearrangements and tyrosine phosphorylation of host cell proteins. Proc Natl Acad Sci U S A 93: 1259-1264.
34. Gutierrez O, Pipaon C, Inohara N, Fontalba A, Ogura Y, et al. (2002) Induction of Nod2 in myelomonocytic and intestinal epithelial cells via nuclear factor-kappa B activation. J Biol Chem 277: 41701-41705.
35. Rosenstiel P, Fantini M, Brautigam K, Kuhbacher T, Waetzig G H, et al. (2003) TNF-alpha and IFN-gamma regulate the expression of the NOD2 (CARD 15) gene in human intestinal epithelial cells. Gastroenterology 124: 1001-1009.
36. Saito H, Kasajima T, Masuda A, Imai Y, Ishikawa M (1988) Lysozyme localization in human gastric and duodenal epithelium. An immunocytochemical study. Cell Tissue Res 251: 307-313.
37. Hidaka E, Ota H, Hidaka H, Hayama M, Matsuzawa K, et al. (2001) Helicobacter pylori and two ultrastructurally distinct layers of gastric mucous cell mucins in the surface mucous gel layer. Gut 49: 474-480.
38. Lecuit M, Abachin E, Martin A, Poyart C, Pochart P, et al. (2004) Immunoproliferative small intestinal disease associated with Campylobacter jejuni. N Engl J Med 350: 239248.
39. Casadaban M J, Cohen S N (1980) Analysis of gene control signals by DNA fusion and cloning in Escherichia coli. J Mol Biol 138: 179-207.
40. Tomb J F, White O, Kerlavage A R, Clayton R A, Sutton G G, et al. (1997) The complete genome sequence of the gastric pathogen Helicobacter pylori. Nature 388: 539-547.
41. Bury-Mone S, Thiberge J M, Contreras M, Maitoumam A, Labigne A, et al. (2004) Responsiveness to acidity via metal ion regulators mediates virulence in the gastric pathogen Helicobacter pylori. Mol Microbiol 53: 623-638.
42. Girardin S E, Tournebize R, Mavris M, Page A L, Li X, et al. (2001) CARD4/Nod1 mediates NF-kappaB and JNK activation by invasive Shigella flexneri. EMBO Rep 2: 736-742.
43. Skouloubris S, Thiberge J M, Labigne A, De Reuse H (1998) The Helicobacter pylori UreI protein is not involved in urease activity but is essential for bacterial survival in vivo. Infect Immun 66: 4517-4521.
44. Derbise A, Lesic B, Dacheux D, Ghigo J M, Carniel E (2003) A rapid and simple method for inactivating chromosomal genes in Yersinia. FEMS Immunol Med Microbiol 38: 113-116.
45. Chevalier C, Thiberge J M, Ferrero R L, Labigne A (1999) Essential role of Helicobacter pylori gamma-glutamyltranspeptidase for the colonization of the gastric mucosa of mice. Mol Microbiol 31: 1359-1372.
46. Glauner B (1988) Separation and quantification of muropeptides with high-performance liquid chromatography. Anal Biochem 172: 451-464.
47. Stenbak C R, Ryu J H, Leulier F, Pili-Floury S, Parquet C, et al. (2004) Peptidoglycan molecular requirements allowing detection by the Drosophila immune deficiency pathway. J Immunol. 173(12):7339-7348.
48. Antignac A, Rousselle J C, Namane A, Labigne A, Taha M K, et al. (2003) Detailed structural analysis of the peptidoglycan of the human pathogen Neisseria meningitidis. J Biol Chem 278: 31521-31528.
49. Dementin S, Bouhss A, Auger G, Parquet C, Mengin-Lecreulx D, et al. (2001) Evidence of a functional requirement for a carbamoylated lysine residue in MurD, MurE and MurF synthetases as established by chemical rescue experiments. Eur J Biochem 268: 58005807.
50. Inohara N, Koseki T, del Peso L, Hu Y, Yee C, et al. (1999) Nod1, an Apaf-1-like activator of caspase-9 and nuclear factor-kappaB. J Biol Chem 274: 14560-14567.
51. Inohara N, Ogura Y, Chen F F, Muto A, Nunez G (2001) Human Nod1 confers responsiveness to bacterial lipopolysaccharides. J. Biol. Chem. 276: 2551-2554.

### REFERENCES FOR SECTION 2

Alm, R. A., Ling, L. S., Moir, D. T., King, B. L., Brown, E. D., Doig, P. C., Smith, D. R., Noonan, B., Guild, B. C., deJonge, B. L., Carmel, G., Tummino, P. J., Caruso, A., Uria437 Nickelsen, M., Mills, D. M., Ives, C., Gibson, R., Merberg, D., Mills, S. D., Jiang, Q., Taylor, D. E., Vovis, G. F., and Trust, T. J. (1999) Genomic-sequence comparison of two unrelated isolates of the human gastric pathogen Helicobacter pylori. Nature 397: 176-180.
Antignac, A., Rousselle, J. C., Namane, A., Labigne, A., Taha, M. K., and Boneca, I. G. (2003) Detailed structural analysis of the peptidoglycan of the human pathogen Neisseria meningitidis. J Biol Chem 278: 31521-31528.
Boneca, I. G., Huang, Z. H., Gage, D. A., and Tomasz, A. (2000) Characterization of Staphylococcus aureus cell wall glycan strands, evidence for a new beta-N-acetylglucosaminidase activity. J Biol Chem 275: 9910-9918.
Boneca, I. G., de Reuse, H., Epinat, J. C., Pupin, M., Labigne, A., and Moszer, I. (2003) A revised annotation and comparative analysis of Helicobacter pylori genomes. Nucleic Acids Res 31: 1704-1714.
Bury-Mone, S., Thiberge, J. M., Contreras, M., Maitournam, A., Labigne, A., and De Reuse, H. (2004) Responsiveness to acidity via metal ion regulators mediates virulence in the gastric pathogen Helicobacter pylori. Mol Microbiol 53: 623-638.
Casadaban, M. J., and Cohen, S. N. (1980) Analysis of gene control signals by DNA fusion and cloning in Escherichia coli. J Mol Biol 138: 179-207.
Costa, K., Bacher, G., Allmaier, G., Dominguez-Bello, M. G., Engstrand, L., Falk, P., de Pedro, M. A., and Garcia-del Portillo, F. (1999) The morphological transition of Helicobacter pylori cells from spiral to coccoid is preceded by a substantial modification of the cell wall. J Bacteriol 181: 3710-3715.
Ferrero, R. L., Thiberge, J. M., Kansau, I., Wuscher, N., Huerre, M., and Labigne, A. (1995) The GroES homolog of Helicobacter pylori confers protective immunity against mucosal infection in mice. Proc Natl Acad Sci U S A 92: 6499-6503.
Ferrero, R. L., Thiberge, J. M., Huerre, M., and Labigne, A. (1998) Immune responses of specific-pathogen-free mice to chronic Helicobacter pylori (strain SS1) infection. Infect Immun 66: 1349-1355.
Glauner, B. (1988) Separation and quantification of muropeptides with high-performance liquid chromatography. Anal Biochem 172: 451-464.
Glauner, B., Holtje, J. V., and Schwarz, U. (1988) The composition of the murein of Escherichia coli. J Biol Chem 263: 10088-10095.
Harz, H., Burgdorf, K., and Holtje, J. V. (1990) Isolation and separation of the glycan strands from murein of Escherichia coli by reversed-phase high-performance liquid chromatography. Anal Biochem 190: 120-128.
Heidrich, C., Templin, M. F., Ursinus, A., Merdanovic, M., Berger, J., Schwarz, H., de Pedro, M. A., and Holtje, J. V. (2001) Involvement of N-acetylmuramyl-L-alanine amidases in cell separation and antibiotic-induced autolysis of Escherichia coli. Mol Microbiol 41: 167-178.
Ishidate, K., Ursinus, A., Holtje, J. V., and Rothfield, L. (1998) Analysis of the length distribution of murein glycan strands in ftsZ and ftsI mutants of E. coli. FEMS Microbiol Lett 168: 71-75.
Israel, D. A., Salama, N., Arnold, C. N., Moss, S. F., Ando, T., Wirth, H. P., Tham, K. T., Camorlinga, M., Blaser, M. J., Falkow, S., and Peek, R. M., Jr. (2001) Helicobacter pylori strain-specific differences in genetic content, identified by microarray, influence host inflammatory responses. J Clin Invest 107: 611-620.
Londono-Arcila, P., Freeman, D., Kleanthous, H., O'Dowd, A. M., Lewis, S., Turner, A. K., Rees, E. L., Tibbitts, T. J., Greenwood, J., Monath, T. P., and Darsley, M. J. (2002) Attenuated Salmonella enterica serovar Typhi expressing urease effectively immunizes mice against Helicobacter pylori challenge as part of a heterologous mucosal priming-parenteral boosting vaccination regimen. Infect Immun 70: 5096488 5106.
Ottemann, K. M., and Lowenthal, A. C. (2002) Helicobacter pylori uses motility for initial colonization and to attain robust infection. Infect Immun 70: 1984-1990.
Quintela, J. C., Caparros, M., and de Pedro, M. A. (1995) Variability of peptidoglycan structural parameters in gram-negative bacteria. FEMS Microbiol Lett 125: 95-100.
Skouloubris, S., Thiberge, J. M., Labigne, A., and De Reuse, H. (1998) The Helicobacter pylori UreI protein is not involved in urease activity but is essential for bacterial survival in vivo. Infect Immun 66: 4517-4521.
Tomasz, A., Albino, A., and Zanati, E. (1970) Multiple antibiotic resistance in a bacterium with suppressed autolytic system. Nature 227: 138-140.
Tomb, J. F., White, O., Kerlavage, A. R., Clayton, R. A., Sutton, G. G., Fleischmann, R. D., Ketchum, K. A., Klenk, H. P., Gill, S., Dougherty, B. A., Nelson, K., Quackenbush, J., Zhou, L., Kirkness, E. F., Peterson, S., Loftus, B., Richardson, D., Dodson, R., Khalak, H. G., Glodek, A., McKenney, K., Fitzegerald, L. M., Lee, N., Adams, M. D., Venter, J. C., and et al. (1997) The complete genome sequence of the gastric pathogen Helicobacter pylori. Nature 388: 539-547.
Travassos, L. H., Girardin, S. E., Philpott, D. J., Blanot, D., Nahori, M. A., Werts, C., and Boneca, I. G. (2004) Toll-like receptor 2-dependent bacterial sensing does not occur via peptidoglycan recognition. EMBO Rep 5: 1000-1006.
Vollmer, W., and Holtje, J. V. (2004) The architecture of the murein (peptidoglycan) in gram negative bacteria: vertical scaffold or horizontal layer(s)? J. Bacteriol. 186: 5978-5987.
Wang, Z. M., Li, X., Cocklin, R. R., Wang, M., Wang, M., Fukase, K., Inamura, S., Kusumoto,
S., Gupta, D., and Dziarski, R. (2003) Human peptidoglycan recognition protein-L is an N-acetylmuramoyl-L-alanine amidase. J. Biol. Chem. 278: 49044-49052.

### REFERENCES FOR SECTION 3

1. Tomb, J. F., et al., Nature 388, 539-547 (1997)
2. Boneca, I. G., et al., Nucleic Acids Chem 278, 31521-31528 (2003)
3. Alm, R. A., et al., Nature 397, 176-180. (1999)
4. Casadaban, M. J., et al., J. Bacteriol. 278, 49044-49052 (1980)
5. Ferrero, R. L., et al., J. Bacteriol. 174, 4212-4217 (1992)
6. Bury-Mone, S., et al., Mol. Microbiol. 53, 623-638 (2004)
7. Skuoubris, S., et al., Infect Immun 66, 4517-4521 (1998)
8. Jenks, P. J., et al., Res. Microbiol. 152 725-734 (20,
9. Bury-Mone, S., et al., Infect Immun 71, 5613-5622 (2003)
10. Edwards, N. J., Mol Microbiol 35, 1530-1539 (2000)
11. Lesse, A. J., et al., J Immunol Methods 126, 109-117 (1990)
12. Tsai, C. M., et al., Anal Biochem 119, 115-119 (1982)
13. Glauner, B. Anal Biochem. 172:451-464 (1988)
14. Antignac, A., et al., J. Biol. Chem. 278, 31521-31528 (2003)
15. Harz, H., et al., Anal Biochem 190, 120-128 (1990)
16. Boneca, I. G., et al., J., Biol Chem 275, 9910-9918 (2000)
17. Labigne, A., et al., Res Microbiol 143, 15-26 (1992)
18. Wang, Z. M., et al., J Biol Chem 278, 49044-49052 (2003)
19. Romeis, T., et al., FEMS Microbiol Lett 111, 141-146 (1993)

### Modifications and Other Embodiments

Various modifications and variations of the described compositions and their methods of use as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the medical, microbiological, biochemical, immunological, pharmaceutical, biological, chemical or related fields are intended to be within the scope of the following claims.

Incorporation by Reference

Each document, patent, patent application or patent publication cited by or referred to in this disclosure is incorporated by reference in its entirety. However, no admission is made that any such reference constitutes prior art and the right to challenge the accuracy and pertinency of the cited documents is reserved.

1. A method for identifying a compound which modulates the pathogenesis of Helicobacter pylori infection by affecting the synthesis or assembly of the peptidoglycan PG layer, comprising:
contacting a test compound with Helicobacter pylori or one or more components of Helicobacter pylori or analog(s) thereof, and
determining the effects on said compound on peptidoglycan structure, on the rate of peptidoglycan synthesis, or on the expression of the amiA, slt or MltD gene(s), compared to a control to which the test compound has not been added.

2. The method of claim 1, wherein said Helicobacter pylori component or analog thereof is a lytic transglycosylase.

3. The method of claim 1, wherein said Helicobacter pylori component is a lytic transglycosylase selected from the group consisting of Slt and MltD, and said method comprises
contacting a test compound with an Slt or MltD protein, and
determining the amount of transglycosylase activity compared to the amount of transglycosylase activity in a control to which the test compound has not been added.

4. The method of claim 1, wherein said test compound is contacted with an Slt or MltD protein in Helicobacter pylori and wherein said determining comprises measuring peptidoglycan degradation in Helicobacter pylori.

5. The method of claim 1, wherein said test compound is contacted with an Slt or MltD protein in Helicobacter pylori, and wherein said determining involves measuring morphological change in Helicobacter pylori, its ability to adhere to, invade, or colonize mammalian cells, its motility, or its replication rate.

6. The method of claim 1, wherein said test compound is contacted with an Slt or MltD protein in Helicobacter pylori and wherein said determining comprises measuring the length of glycan chains.

7. The method of claim 1, wherein said test compound is contacted with an Slt or MltD protein in Helicobacter pylori and wherein said determining further comprises measuring the NF-κB and/or IL-8 activity.

8. The method of claim 1, wherein said test compound is contacted with Slt or MltD protein in Helicobacter pylori and wherein said determining comprises measuring the motility of Helicobacter pylori.

9. The method of claim 1, wherein said test compound is contacted with an Slt or MltD protein in Helicobacter pylori and wherein said determining comprises measuring the bacteriostatic or bacteriocidal effects of said compound.

10. The method of claim 1, wherein said one or more components of Helicobacter pylori is an Slt protein encoded by SEQ ID NO: 1 or an analog thereof which is encoded by a polynucleotide which is at least 90-95% similar to SEQ ID NO: 1 or which is encoded by a polynucleotide which hybridizes under stringent conditions to the complement of SEQ ID NO: 1, wherein stringent conditions comprising hybridization at 50-68° C. and washing in 0.1×SSC at 50-68° C.

11. The method of claim 1, wherein said one or more components of Helicobacter pylori is an MltD protein encoded by SEQ ID NO: 3 or an analog thereof which is encoded by a polynucleotide which is at least 90-95% similar to SEQ ID NO: 3 or which is encoded by a polynucleotide which hybridizes under stringent conditions to the complement of SEQ ID NO: 3, wherein stringent conditions comprising hybridization at 50-68° C. and washing in 0.1×SSC at 50-68° C.

12. The method of claim 1, wherein said Helicobacter pylori component or analog thereof is an N-acetylmuramoyl-L-alanylamidase.

13. The method of claim 1, wherein said Helicobacter pylori component is an N-acetylmuramoyl-L-alanylamidase which is AmiA, and said method comprises
contacting a test compound with an AmiA protein, and
determining the amount of N-acetylmuramoyl-L-alanylamidase activity compared to the amount of N-acetylmuramoyl-L-alanylamidase activity in a control to which the test compound has not been added.

14. The method of claim 1, wherein said test compound is contacted with an AmiA protein in Helicobacter pylori and wherein said determining comprises measuring peptidoglycan degradation in Helicobacter pylori.

15. The method of claim 1, wherein said test compound is contacted with an AmiA protein in Helicobacter pylori, and wherein said determining involves measuring morphological change in Helicobacter pylori, its ability to adhere to, invade, or colonize mammalian cells, its motility, or its replication rate.

16. The method of claim 1, wherein said test compound is contacted with an AmiA protein in Helicobacter pylori and wherein said determining comprises measuring the length of glycan chains.

17. The method of claim 1, wherein said test compound is contacted with an AmiA protein in Helicobacter pylori and wherein said determining further comprises measuring the NP-κB and/or IL-8 activity.

18. The method of claim 1, wherein said test compound is contacted with Ami A protein in Helicobacter pylori and wherein said determining comprises measuring cell division and/or morphological transition of Helicobacter pylori from a spiral to coccoid morphology.

19. The method of claim 1, wherein said test compound is contacted with an Ami A protein in Helicobacter pylori and wherein said determining comprises measuring the bacteriostatic or bacteriocidal effects of said compound.

20. The method of claim 1, wherein said one or more components of Helicobacter pylori is an AmiA protein encoded by SEQ ID NO: 5 or an analog thereof which is encoded by a polynucleotide which is at least 90-95% similar to SEQ ID NO: 5 or which is encoded by a polynucleotide which hybridizes under stringent conditions to the complement of SEQ ID NO: 5, wherein stringent conditions comprising hybridization at 50-68° C. and washing in 0.1×SSC at 50-68° C.

21. A method for identifying a compound which modulates the pathogenesis of Helicobacter pylori infection by affecting the synthesis or assembly of the peptidoglycan PG layer, comprising:
contacting a test compound with one or more components of Helicobacter pylori or analog(s) thereof, and
determining a change in the level of the expression of a gene selected from the group consisting of slt, mltD and amiA.
